(19) United States
(12) Patent Application Publication
(10) Pub. No.: US 2025/0282760 A1
(43) Pub. Date: Sep. 11, 2025

(54) OREXIN RECEPTOR AGONISTS

(71) Applicant: Merck Sharp & Dohme LLC, Rahway, NJ (US)

(72) Inventors: Jeffrey C. Kern, Gilbertsville, PA (US); Michael T. Rudd, Collegeville, PA (US)

(73) Assignee: Merck Sharp & Dohme LLC, Rahway, NJ (US)

(21) Appl. No.: 18/862,822

(22) PCT Filed: May 1, 2023
(86) PCT No.: PCT/US2023/020515

§ 371 Date: Nov. 4, 2024

Related U.S. Application Data
(60) Provisional application No. 63/339,058, filed on May 6, 2022.

(51) Int. Cl. C07D 403/12; A61K 31/496; A61K 31/497; A61K 31/506; C07D 241/04; C07D 401/12; C07D 405/06; C07D 405/12; C07D 405/14; C07D 409/06; C07D 413/12; C07D 417/06; C07D 417/12

(52) U.S. Cl. CPC C07D 403/12; A61K 31/496; A61K 31/497; A61K 31/506; C07D 241/04; C07D 401/12; C07D 405/06; C07D 405/12; C07D 405/14; C07D 409/06; C07D 413/12; C07D 417/06; C07D 417/12

(57) ABSTRACT

The present invention is directed to compounds of Formula I which are agonists of orexin receptors. The present invention is also directed to uses of the compounds described herein in the potential treatment or prevention of neurological and psychiatric disorders and diseases in which orexin receptors are involved. The present invention is also directed to compositions comprising these compounds. The present invention is also directed to uses of these compositions in the potential prevention or treatment of such diseases in which orexin receptors are involved.

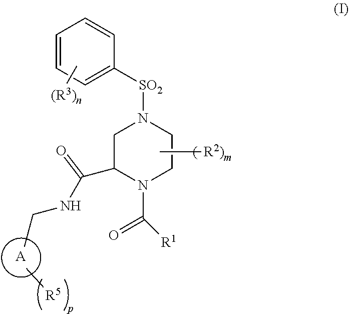

### BACKGROUND OF THE INVENTION

The orexins (hypocretins) comprise two neuropeptides produced in the hypothalamus: orexin A (OX-A) (a 33 amino acid peptide) and the orexin B (OX-B) (a 28 amino acid peptide) (Sakurai T. et al., Cell, 1998, 92, 573-585). Orexins regulate states of sleep and wakefulness opening potentially novel therapeutic approaches for narcolepsy, idiopathic hypersomnia, excessive daytime sleepiness, shift work disorder, obstructive sleep apnea and insomnia (Chemelli R. M. et al., Cell, 1999, 98, 437-451). Orexins are found to stimulate food consumption in rats suggesting a physiological role for these peptides as mediators in the central feedback mechanism that regulates feeding behavior (Sakurai T. et al., Cell, 1998, 92, 573-585). Orexins have also been indicated as playing a role in arousal, emotion, energy homeostasis, reward, learning and memory (Peyron, et al., Journal Neurosci., 1998, 18(23):9996-100150, Harris, et al., Trends Neurosci., 2006, 29 (10), 571-577). Two orexin receptors have been cloned and characterized in mammals. They belong to the super family of G-protein coupled receptors (Sakurai T. et al., Cell, 1998, 92, 573-585): the orexin-1 receptor (OX or OX1R) is partially selective for OX-A and the orexin-2 receptor (OX2 or OX2R) is capable of binding OX-A as well as OX-B with similar affinity. The physiological actions in which orexins are presumed to participate are thought to be expressed via one or both of OX1 receptor and OX2 receptor as the two subtypes of orexin receptors.

### SUMMARY OF THE INVENTION

The present invention is directed to sulfonamide compounds which are agonists of orexin receptors. The present invention is also directed to uses of the compounds described herein in the potential treatment or prevention of neurological and psychiatric disorders and diseases in which orexin receptors are involved. The present invention is also directed to compositions comprising these compounds. The present invention is also directed to uses of these compositions in the potential prevention or treatment of such diseases in which orexin receptors are involved.

### DETAILED DESCRIPTION OF THE INVENTION

The present invention is directed to compounds of the formula I:

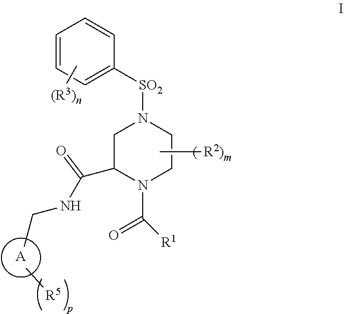

wherein:
Ring A is selected from phenyl, naphthyl and heteroaryl;
R is independently selected from H, —C₁₋₆alkyl, O—C₁₋₆alkyl, CF₃, OCF₂CH₃, OCF₂CH₂CH₃, and CH₂CF₂CH₃;
R¹ is selected from:
   (1) C₁₋₆ alkyl, where the alkyl is unsubstituted or substituted with one to six substituents independently selected from R⁴,
   (2) C₃₋₆ cycloalkyl, where the cycloalkyl is unsubstituted or substituted with one to six substituents independently selected from R⁴,
   (3) aryl, where the aryl is unsubstituted or substituted with one to three substituents independently selected from R⁴; and
   (4) heteroaryl, where the heteroaryl is unsubstituted or substituted with one to three substituents independently selected from R⁴;
R² is independently selected from:
   (1) H, and
   (2) C₁₋₆alkyl, where the alkyl is unsubstituted or substituted with one to six substituents selected from halo, CF₃ and —C₁₋₆alkyl;
R³ is independently selected from:
   (1) C₁₋₆alkyl, where the alkyl is unsubstituted or substituted with one to six substituents independently selected from R⁴,
   (2) OR,
   (3) halo, and
   (4) CN;
R⁴ is independently selected from:
   (1) hydrogen,
   (2) C₁₋₆alkyl, which is unsubstituted or substituted with one to six substituents independently selected from R⁶,
   (3) OR,
   (4) CN,
   (5) halo,
   (6) aryl, which is unsubstituted or substituted with one to six substituents independently selected from R⁶, and
   (7) heteroaryl, which is unsubstituted or substituted with one to six substituents independently selected from R⁶;
R⁵ is independently selected from:
   (1) hydrogen,
   (2) C₁₋₆alkyl, which is unsubstituted or substituted with one to six substituents independently selected from R⁶,
   (3) halo,
   (4) CN,
   (5) aryl, which is unsubstituted or substituted with one to six substituents independently selected from R⁶,
   (6) OR,
   (7) heteroaryl, which is unsubstituted or substituted with one to six substituents independently selected from R⁶,
   (8) OCF₃,
   (9) OCF₂CH₃,
   (10) OCF₂CH₂CH₃, and
   (11) CH₂CF₂CH₃;
R⁶ is independently selected from: H, halo, OR, CF₃, and C₁₋₆alkyl;
m is selected from 0, 1, 2 and 3;
n is selected from 0, 1 and 2; and
p is selected from 0, 1, 2, 3, and 4;
or a pharmaceutically acceptable salt thereof.

In another embodiment, the instant invention is directed to compounds of the formula 1, wherein:
R¹ is selected from:
   (1) C₁₋₆alkyl, where the alkyl is unsubstituted or substituted with one to six substituents independently selected from R⁴,
   (2) phenyl, where the phenyl is unsubstituted or substituted with one to three substituents independently selected from R⁴; and
   (3) heteroaryl, where the heteroaryl is selected from thiophenyl, thiazolyl, or furanyl, which is unsubstituted or substituted with one to three substituents independently selected from R⁴;
R² is independently selected from:
   (1) H, and
   (2) C₁₋₆alkyl, where the alkyl is unsubstituted or substituted with one to six substituents selected from halo, CF₃ and —C₁₋₆alkyl;
R³ is independently selected from:
   (1) C₁₋₆alkyl, where the alkyl is unsubstituted or substituted with one to six substituents independently selected from R⁴,
   (2) OR,
   (3) halo, and
   (4) CN;
R⁴ is independently selected from:
   (1) hydrogen,
   (2) C₁₋₆alkyl, which is unsubstituted or substituted with one to six substituents independently selected from R⁶,
   (3) OR,
   (4) CN,
   (5) halo,
   (6) aryl, which is unsubstituted or substituted with one to six substituents independently selected from R⁶, and
   (7) heteroaryl, which is unsubstituted or substituted with one to six substituents independently selected from R⁶;
R⁵ is independently selected from:
   (1) hydrogen,
   (2) C₁₋₆alkyl, which is unsubstituted or substituted with one to six substituents independently selected from R⁶,
   (3) halo,
   (4) CN,
   (5) aryl, which is unsubstituted or substituted with one to six substituents independently selected from R⁶,
   (6) OR,
   (7) heteroaryl, which is unsubstituted or substituted with one to six substituents independently selected from R⁶,
   (8) OCF₃,
   (9) OCF₂CH₃,
   (10) OCF₂CH₂CH₃, and
   (11) CH₂CF₂CH₃;
R⁶ is independently selected from: H, halo, OR, CF₃, and C₁₋₆alkyl;
m is selected from 0 or 1;
n is selected from 0, 1 or 2; and
p is selected from 0, 1, 2, or 3;
or a pharmaceutically acceptable salt thereof.

Another embodiment of the instant invention is directed to compounds of the formula IA:

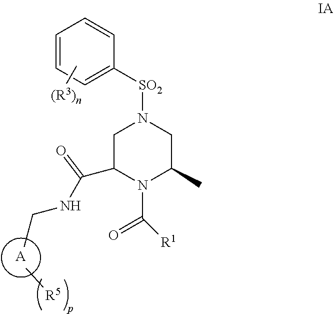

wherein:
Ring A is selected from phenyl, naphthyl and heteroaryl;
R is independently selected from H, C₁₋₆alkyl, —O—C₁₋₆alkyl, CF₃, OCF₂CH₃, OCF₂CH₂CH₃, and CH₂CF₂CH₃;
R¹ is selected from:
   (1) C₁₋₆ alkyl, where the alkyl is unsubstituted or substituted with one to six substituents independently selected from R⁴,
   (2) C₃₋₆ cycloalkyl, where the cycloalkyl is unsubstituted or substituted with one to six substituents independently selected from R⁴,
   (3) aryl, where the aryl is unsubstituted or substituted with one to three substituents independently selected from R⁴; and
   (4) heteroaryl, where the heteroaryl is unsubstituted or substituted with one to three substituents independently selected from R⁴;
R³ is independently selected from:
   (1) C₁₋₆alkyl, where the alkyl is unsubstituted or substituted with one to six substituents independently selected from R⁴,
   (2) OR,
   (3) halo, and
   (4) CN;
R⁴ is independently selected from:
   (1) hydrogen,
   (2) C₁₋₆alkyl, which is unsubstituted or substituted with one to six substituents independently selected from R⁶,
   (3) OR,
   (4) CN,
   (5) halo,
   (6) aryl, which is unsubstituted or substituted with one to six substituents independently selected from R⁶, and
   (7) heteroaryl, which is unsubstituted or substituted with one to six substituents independently selected from R⁶;
R⁵ is independently selected from:
   (1) hydrogen,
   (2) C₁₋₆alkyl, which is unsubstituted or substituted with one to six substituents independently selected from R⁶,
   (3) halo,
   (4) CN,
   (5) aryl, which is unsubstituted or substituted with one to six substituents independently selected from R⁶,
   (6) OR,
   (7) heteroaryl, which is unsubstituted or substituted with one to six substituents independently selected from R⁶,
   (8) OCF₃,
   (9) OCF₂CH₃,
   (10) OCF₂CH₂CH₃, and
   (11) CH₂CF₂CH₃;
R⁶ is independently selected from: H, halo, OR, CF₃, and C₁₋₆alkyl;
n is selected from 0, 1 and 2; and
p is selected from 0, 1, 2, 3, and 4;
or a pharmaceutically acceptable salt thereof.

Another embodiment of the instant invention is directed to compounds of the formula IB:

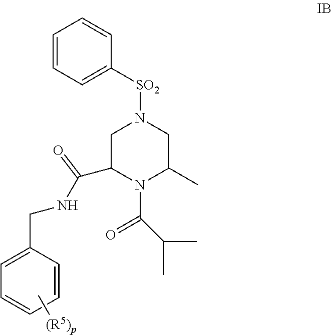

wherein:
R is independently selected from H, C₁₋₆alkyl, O—C₁₋₆alkyl, CF₃, OCF₂CH₃, OCF₂CH₂CH₃, and CH₂CF₂CH₃;
R⁵ is independently selected from:
   (1) hydrogen,
   (2) C₁₋₆alkyl, which is unsubstituted or substituted with one to six substituents independently selected from R⁶,
   (3) halo,
   (4) CN,
   (5) aryl, which is unsubstituted or substituted with one to six substituents independently selected from R⁶,
   (6) OR,
   (7) heteroaryl, which is unsubstituted or substituted with one to six substituents independently selected from R⁶,
   (8) OCF₃,
   (9) OCF₂CH₃,
   (10) OCF₂CH₂CH₃, and
   (11) CH₂CF₂CH₃;
R⁶ is independently selected from: H, halo, OR, CF₃, and C₁₋₆alkyl; and
p is selected from 0, 1, 2, 3, or 4;
or a pharmaceutically acceptable salt thereof.

It is understood that reference herein to “compounds of Formula I” also encompasses compounds of Formula IA and Formula IB, unless otherwise indicated.

An embodiment of the present invention includes compounds of Formula I wherein Ring A is selected from: phenyl, naphthyl, pyridinyl, pyrazinyl, or pyrimidinyl. A further embodiment of the present invention includes compounds wherein Ring A is selected from: phenyl, naphthyl, or pyridinyl. In an embodiment, Ring A is phenyl. In an embodiment, Ring A is naphthyl. In an embodiment, Ring A is pyridinyl.

An embodiment of the present invention includes compounds of Formula I wherein R¹ is selected from: C₁₋₆alkyl or phenyl, where the alkyl or phenyl is unsubstituted or substituted with one to six substituents independently selected from R⁴.

An embodiment of the present invention includes compounds of Formula I wherein R² is C₁₋₆alkyl, where the alkyl is unsubstituted or substituted with one to six substituents selected from halo, CF₃ and C₁₋₆alkyl.

An embodiment of the present invention includes compounds of Formulae I or IA wherein R⁵ is independently selected from C₁₋₆alkyl, phenyl, OR, heteroaryl, OCF₃, OCF₂CH₃, OCF₂CH₂CH₃, or CH₂CF₂CH₃, wherein said alkyl, phenyl or heteroaryl is unsubstituted or substituted with one to six substituents independently selected from R⁶.

An embodiment of the present invention includes compounds of Formula I wherein m is selected from 0 or 1. An embodiment of the present invention includes compounds of Formula I wherein m is 0. An embodiment of the present invention includes compounds of Formula I wherein m is 1.

An embodiment of the present invention includes compounds of Formula I wherein n is selected from 0 or 1. An embodiment of the present invention includes compounds of Formula I wherein n is 0. An embodiment of the present invention includes compounds of Formula I wherein n is 1.

An embodiment of the present invention includes compounds of Formula I wherein p is selected from 0, 1, 2 or 3. An embodiment of the present invention includes compounds of Formula I wherein p is 0, 1 or 2. An embodiment of the present invention includes compounds of Formula I wherein n p is 1 or 2. I An embodiment of the present invention includes compounds of Formula I wherein p is 1.

Certain embodiments of the present invention include a compound which is selected from the group consisting of the subject compounds of the Examples herein or a pharmaceutically acceptable salt thereof.

Certain embodiments of the present invention include a compound which is selected from:
N-(naphthalen-2-ylmethyl)-4-(phenylsulfonyl)-1-(thiophene-2-carbonyl)piperazine-2-carboxamide (6);
1-(2-methylbutanoyl)-N-(naphthalen-2-ylmethyl)-4-(phenylsulfonyl)piperazine-2-carboxamide (10-1);
N-(naphthalen-2-ylmethyl)-4-(phenylsulfonyl)-1-propionylpiperazine-2-carboxamide (10-2);
1-butyryl-N-(naphthalen-2-ylmethyl)-4-(phenylsulfonyl)piperazine-2-carboxamide (10-3)
1-isobutyryl-N-(naphthalen-2-ylmethyl)-4-(phenylsulfonyl)piperazine-2-carboxamide (10-4);
1-(cyclopropanecarbonyl)-N-(naphthalen-2-ylmethyl)-4-(phenylsulfonyl)piperazine-2-carboxamide (10-5);
1-(cyclobutanecarbonyl)-N-(naphthalen-2-ylmethyl)-4-(phenylsulfonyl)piperazine-2-carboxamide (10-6);
N-(naphthalen-2-ylmethyl)-4-(phenylsulfonyl)-1-(thiazole-2-carbonyl)piperazine-2-carboxamide (10-7);
N-(naphthalen-2-ylmethyl)-4-(phenylsulfonyl)-1-(thiophene-3-carbonyl)piperazine-2-carboxamide (10-8);
1-(furan-2-carbonyl)-N-(naphthalen-2-ylmethyl)-4-(phenylsulfonyl)piperazine-2-carboxamide (10-9);
N-(naphthalen-2-ylmethyl)-4-(phenylsulfonyl)-1-(thiazole-5-carbonyl)piperazine-2-carboxamide (10-10);
1-(2-methylbenzoyl)-N-(naphthalen-2-ylmethyl)-4-(phenylsulfonyl)piperazine-2-carboxamide (10-11);
1-(3-methylthiophene-2-carbonyl)-N-(naphthalen-2-ylmethyl)-4-(phenylsulfonyl)piperazine-2-carboxamide (10-12);
1-acetyl-N-(naphthalen-2-ylmethyl)-4-(phenylsulfonyl)piperazine-2-carboxamide (11);
cis-1-isobutyryl-6-methyl-N-(naphthalen-2-ylmethyl)-4-(phenylsulfonyl)piperazine-2-carboxamide (19-1);
cis-N-(4-(furan-2-yl)benzyl)-1-isobutyryl-6-methyl-4-(phenylsulfonyl)piperazine-2-carboxamide (19-2);
(2R,6R)—N-(4-(furan-2-yl)benzyl)-1-isobutyryl-6-methyl-4-(phenylsulfonyl)piperazine-2-carboxamide (19-2-1 PEAK 1);
(2S,6S)—N-(4-(furan-2-yl)benzyl)-1-isobutyryl-6-methyl-4-(phenylsulfonyl)piperazine-2-carboxamide (19-2-2, PEAK 2);
cis-N-([1,1′-biphenyl]-4-ylmethyl)-1-isobutyryl-6-methyl-4-(phenylsulfonyl)piperazine-2-carboxamide (19-3);
cis-N-(4-(1,1-difluoroethoxy)benzyl)-1-isobutyryl-6-methyl-4-(phenylsulfonyl)piperazine-2-carboxamide (19-4);
(2R,6R)—N-(4-(1,1-difluoroethoxy)benzyl)-1-isobutyryl-6-methyl-4-(phenylsulfonyl)piperazine-2-carboxamide (19-4-1, PEAK 1);
(2S,6S)—N-(4-(1,1-difluoroethoxy)benzyl)-1-isobutyryl-6-methyl-4-(phenylsulfonyl)piperazine-2-carboxamide (19-4-2, PEAK 2);
cis-1-isobutyryl-6-methyl-4-(phenylsulfonyl)-N-(4-(pyrimidin-2-yl)benzyl)piperazine-2-carboxamide (19-5);
cis-1-isobutyryl-6-methyl-4-(phenylsulfonyl)-N-(4-(thiazol-2-yl)benzyl)piperazine-2-carboxamide (19-6);
cis-N-(4-(2H-1,2,3-triazol-2-yl)benzyl)-1-isobutyryl-6-methyl-4-(phenylsulfonyl)piperazine-2-carboxamide (19-7);
cis-1-isobutyryl-6-methyl-N-(4-(oxazol-2-yl)benzyl)-4-(phenylsulfonyl)piperazine-2-carboxamide (19-8);
cis-N-(4-(1H-pyrazol-1-yl)benzyl)-1-isobutyryl-6-methyl-4-(phenylsulfonyl)piperazine-2-carboxamide (19-9);
cis-1-isobutyryl-6-methyl-4-(phenylsulfonyl)-N-(4-propylbenzyl)piperazine-2-carboxamide (19-10);
cis-N-(4-ethoxybenzyl)-1-isobutyryl-6-methyl-4-(phenylsulfonyl)piperazine-2-carboxamide (19-11);
cis-N-((2′-fluoro-[1,1′-biphenyl]-4-yl)methyl)-1-isobutyryl-6-methyl-4-(phenylsulfonyl)piperazine-2-carboxamide (21);
cis-N-((5-(furan-2-yl)pyridin-2-yl)methyl)-1-isobutyryl-6-methyl-4-(phenylsulfonyl)piperazine-2-carboxamide (23);
cis-N-(3-fluoro-4-(furan-2-yl)benzyl)-1-isobutyryl-6-methyl-4-(phenylsulfonyl)piperazine-2-carboxamide (26);
cis-N-(4-(furan-2-yl)-2-methylbenzyl)-1-isobutyryl-6-methyl-4-(phenylsulfonyl)piperazine-2-carboxamide (29);
cis-N-(2-fluoro-4-(furan-2-yl)benzyl)-1-isobutyryl-6-methyl-4-(phenylsulfonyl)piperazine-2-carboxamide (32);
cis-N-((5-(furan-2-yl)pyrazin-2-yl)methyl)-1-isobutyryl-6-methyl-4-(phenylsulfonyl)piperazine-2-carboxamide (35);
cis-N-((5-(furan-2-yl)pyrazin-2-yl)methyl)-1-isobutyryl-6-methyl-4-(phenylsulfonyl)piperazine-2-carboxamide (39);
cis-1-isobutyryl-6-methyl-4-(phenylsulfonyl)-N-(4-(pyridin-2-yl)benzyl)piperazine-2-carboxamide (42-1);
1-isobutyryl-6-methyl-4-(phenylsulfonyl)-N-(4-(pyridin-3-yl)benzyl)piperazine-2-carboxamide (42-2);
1-isobutyryl-6-methyl-4-(phenylsulfonyl)-N-(4-(pyridin-4-yl)benzyl)piperazine-2-carboxamide (42-3);
cis-N-(4-(furan-2-yl)-3-methoxybenzyl)-1-isobutyryl-6-methyl-4-(phenylsulfonyl)piperazine-2-carboxamide (47-1);
cis-N-(3-cyano-4-(furan-2-yl)benzyl)-1-isobutyryl-6-methyl-4-(phenylsulfonyl)piperazine-2-carboxamide (47-2);
cis-N-(3,5-difluoro-4-(furan-2-yl)benzyl)-1-isobutyryl-6-methyl-4-(phenylsulfonyl)piperazine-2-carboxamide (47-3);
cis-N-(4-(furan-2-yl)-2-methoxybenzyl)-1-isobutyryl-6-methyl-4-(phenylsulfonyl)piperazine-2-carboxamide (47-4);
cis-N-(5-fluoro-4-(furan-2-yl)-2-methylbenzyl)-1-isobutyryl-6-methyl-4-(phenylsulfonyl) piperazine-2-carboxamide (47-5);
cis-N-(2-ethyl-4-(furan-2-yl)benzyl)-1-isobutyryl-6-methyl-4-(phenylsulfonyl)piperazine-2-carboxamide (47-6);
cis-N-(3-fluoro-4-(furan-2-yl)-2-methylbenzyl)-1-isobutyryl-6-methyl-4-(phenylsulfonyl) piperazine-2-carboxamide (51);
cis-N-((5-(furan-2-yl)-3-methylpyridin-2-yl)methyl)-1-isobutyryl-6-methyl-4-(phenylsulfonyl)piperazine-2-carboxamide (55);
cis-6-methyl-N-(naphthalen-2-ylmethyl)-4-(phenylsulfonyl)-1-(thiophene-2-carbonyl)piperazine-2-carboxamide (57);
cis-N-(4-(1,1-difluoropropoxy)benzyl)-1-isobutyryl-6-methyl-4-(phenylsulfonyl)piperazine-2-carboxamide (61);
cis-N-(4-(1,1-difluoropropoxy)benzyl)-1-isobutyryl-6-methyl-4-(phenylsulfonyl)piperazine-2-carboxamide (66);
cis-6-ethyl-N-(4-(furan-2-yl)benzyl)-1-isobutyryl-4-(phenylsulfonyl)piperazine-2-carboxamide (71);
cis-N-(4-(furan-2-yl)benzyl)-1-isobutyryl-4-(phenylsulfonyl)-6-(trifluoromethyl)piperazine-2-carboxamide (78);
N-(4-(furan-2-yl)benzyl)-1-isobutyryl-6,6-dimethyl-4-(phenylsulfonyl)piperazine-2-carboxamide (83);
N-(naphthalen-2-ylmethyl)-1-(thiophene-2-carbonyl)-4-tosylpiperazine-2-carboxamide (89-1);
4-[(3-methylphenyl)sulfonyl]-N-[(naphthalen-2-yl)methyl]-1-(thiophene-2-carbonyl)piperazine-2-carboxamide (89-2);
4-[(2-methylphenyl)sulfonyl]-N-[(naphthalen-2-yl)methyl]-1-(thiophene-2-carbonyl)piperazine-2-carboxamide (89-3);
4-[(2-chlorophenyl)sulfonyl]-N-[(naphthalen-2-yl)methyl]-1-(thiophene-2-carbonyl)piperazine-2-carboxamide (89-4);
4-[(4-cyanophenyl)sulfonyl]-N-[(naphthalen-2-yl)methyl]-1-(thiophene-2-carbonyl)piperazine-2-carboxamide (89-5);
4-[(3-cyanophenyl)sulfonyl]-N-[(naphthalen-2-yl)methyl]-1-(thiophene-2-carbonyl)piperazine-2-carboxamide (89-6);
4-[(4-methoxyphenyl)sulfonyl]-N-[(naphthalen-2-yl)methyl]-1-(thiophene-2-carbonyl)piperazine-2-carboxamide (89-7);
4-[(3-methoxyphenyl)sulfonyl]-N-[(naphthalen-2-yl)methyl]-1-(thiophene-2-carbonyl)piperazine-2-carboxamide (89-8);
4-[(4-fluorophenyl)sulfonyl]-N-[(naphthalen-2-yl)methyl]-1-(thiophene-2-carbonyl)piperazine-2-carboxamide (89-9);
4-[(3-fluorophenyl)sulfonyl]-N-[(naphthalen-2-yl)methyl]-1-(thiophene-2-carbonyl)piperazine-2-carboxamide (89-10); and
4-[(2-fluorophenyl)sulfonyl]-N-[(naphthalen-2-yl)methyl]-1-(thiophene-2-carbonyl)piperazine-2-carboxamide (89-11);
   or a pharmaceutically acceptable salt thereof.

Further embodiments of the present invention include a compound which is selected from
cis-N-(4-(furan-2-yl)benzyl)-1-isobutyryl-6-methyl-4-(phenylsulfonyl)piperazine-2-carboxamide (19-2);
(2R,6R)—N-(4-(furan-2-yl)benzyl)-1-isobutyryl-6-methyl-4-(phenylsulfonyl)piperazine-2-carboxamide (19-2-1 PEAK 1);
cis-N-([1,1′-biphenyl]-4-ylmethyl)-1-isobutyryl-6-methyl-4-(phenylsulfonyl)piperazine-2-carboxamide (19-3);
cis-N-(4-(1,1-difluoroethoxy)benzyl)-1-isobutyryl-6-methyl-4-(phenylsulfonyl)piperazine-2-carboxamide (19-4);
(2R,6R)—N-(4-(1,1-difluoroethoxy)benzyl)-1-isobutyryl-6-methyl-4-(phenylsulfonyl)piperazine-2-carboxamide (19-4-1, PEAK 1);
cis-1-isobutyryl-6-methyl-4-(phenylsulfonyl)-N-(4-(pyrimidin-2-yl)benzyl)piperazine-2-carboxamide (19-5);
cis-1-isobutyryl-6-methyl-4-(phenylsulfonyl)-N-(4-(thiazol-2-yl)benzyl)piperazine-2-carboxamide (19-6);
cis-N-(4-(2H-1,2,3-triazol-2-yl)benzyl)-1-isobutyryl-6-methyl-4-(phenylsulfonyl)piperazine-2-carboxamide (19-7);
cis-1-isobutyryl-6-methyl-N-(4-(oxazol-2-yl)benzyl)-4-(phenylsulfonyl)piperazine-2-carboxamide (19-8);
cis-1-isobutyryl-6-methyl-4-(phenylsulfonyl)-N-(4-propylbenzyl)piperazine-2-carboxamide (19-10);
cis-N-((2′-fluoro-[1,1′-biphenyl]-4-yl)methyl)-1-isobutyryl-6-methyl-4-(phenylsulfonyl)piperazine-2-carboxamide (21); and
cis-1-isobutyryl-6-methyl-4-(phenylsulfonyl)-N-(4-(pyridin-2-yl)benzyl)piperazine-2-carboxamide (42-1);
   or a pharmaceutically acceptable salt thereof.

Further embodiments of the present invention include a compound which is selected from
cis-N-(4-(furan-2-yl)benzyl)-1-isobutyryl-6-methyl-4-(phenylsulfonyl)piperazine-2-carboxamide (19-2);
(2R,6R)—N-(4-(furan-2-yl)benzyl)-1-isobutyryl-6-methyl-4-(phenylsulfonyl)piperazine-2-carboxamide (19-2-1 PEAK 1);
cis-N-(4-(1,1-difluoroethoxy)benzyl)-1-isobutyryl-6-methyl-4-(phenylsulfonyl)piperazine-2-carboxamide (19-4);
(2R,6R)—N-(4-(1,1-difluoroethoxy)benzyl)-1-isobutyryl-6-methyl-4-(phenylsulfonyl)piperazine-2-carboxamide (19-4-1, PEAK 1); and
cis-1-isobutyryl-6-methyl-4-(phenylsulfonyl)-N-(4-(pyrimidin-2-yl)benzyl)piperazine-2-carboxamide (19-5);
   or a pharmaceutically acceptable salt thereof.

The present invention includes compounds that may contain one or more asymmetric centers. Thus, compounds of the present invention include those that can occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. Likewise, the present invention includes tautomeric forms of the compounds disclosed herein. Formula I shows the structure of the class of compounds without specific stereochemistry. At least some of the chemical names of compounds of the invention as set forth in this application may have been generated on an automated basis by use of commercially available chemical naming software programs, and have not been independently verified.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. Absolute stereochemistry may also be elucidated through other techniques known in the art, such as cryogenic electron microscopy. Relative stereochemistry may be determined using nuclear magnetic resonance with methods known in the art. Stereochemistry may be assigned by analogy to a set of isomers based on their relative biological activity following the same trend established by a similar stereochemically defined group of isomers. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art. Compounds of the present invention may also be separated by supercritical fluid chromatography (SFC). Isomers are named according to the order they came off the column (PEAK 1, PEAK 2, etc.). For isomers that have a designation similar to “PEAK 2, part b” or “PEAK 2b”, one with skill in the art would understand that sometimes the peaks may contain more than a single isomer and when cut in half or fractionated further result in a “part a” and a “part b” of one peak. Furthermore, some separations required multiple rounds of purifications by the same method of purification and/or an alternative purification system to resolve mixtures into single isomers (i.e., PEAK 2 was a mixture of multiple isomers after the initial purification that was resolved further to give PEAK 2A and PEAK 2B). Additionally, a mixture may be a mixture of 2 to 4 stereoisomers. Alternatively, any enantiomer of a compound may be obtained by stereoselective synthesis using optically pure starting materials or reagents of known configuration by methods well known in the art.

As appreciated by those of skill in the art, halogen or halo as used herein are intended to include fluoro, chloro, bromo and iodo. Similarly, C₁₋₆, as in C₁₋₆alkyl is defined to identify the group as having 1, 2, 3, 4, 5 or 6 carbons in a linear or branched arrangement, such that C₁₋₆alkyl specifically includes methyl, ethyl, n-propyl, iso-propyl, n-butyl, iso-butyl, tert-butyl, pentyl, and hexyl. Additionally, heteroaryl, or heterocycles, unless otherwise specified, means mono- and bicyclic saturated or unsaturated rings and ring systems containing at least one heteroatom selected from N, S and O, each of said rings having 3 to 10 atoms in which the point of attachment may be carbon or nitrogen. Examples of “heteroaryl” include, but are not limited to, azetidinyl, oxetanyl, pyrrolidinyl, piperidinyl, piperazinyl, imidazolidinyl, tetrahydrofuranyl, 1,4-dioxanyl, morpholinyl, thiomorpholinyl, tetrahydrothienyl, thiophenyl, thiazolyl, furanyl, pyrazolyl, imidazolyl, oxazolyl, isoxazolyl, isothiazolyl, pyridinyl, pyridazinyl, pyrimidinyl, pyrazinyl, triazinyl, triazolyl and the like. In embodiments of the instant application, heteroaryl may be independently selected from thiophenyl, thiazolyl, furanyl, pyrazolyl, imidazolyl, oxazolyl, isoxazolyl, isothiazolyl, pyridinyl, pyridazinyl, pyrimidinyl, pyrazinyl, triazinyl, or triazolyl. In further embodiments of the instant application, heteroaryl may be independently selected from thiophenyl, thiazolyl, furanyl, pyrazolyl, imidazolyl, oxazolyl, isoxazolyl, isothiazolyl or triazolyl. In embodiments of the instant application, heteroaryl may be independently selected from pyridinyl, pyridazinyl, pyrimidinyl, pyrazinyl, triazinyl, furanyl, triazolyl, thiazolyl or isothiazolyl. Heterocycles can also exist in tautomeric forms, e.g., 2- and 4-pyridones. In some embodiments, heterocyclyl is independently selected from azetidinyl, oxetanyl, piperidinyl, pyridyl, pyrrolidinyl or tetrahydrofuranyl. A group which is designated as being independently substituted with substituents may be independently substituted with multiple numbers of such substituents.

The present invention also includes all pharmaceutically acceptable isotopic variations of a compound of the Formula I in which one or more atoms is replaced by atoms having the same atomic number, but an atomic mass or mass number different from the atomic mass or mass number usually found in nature. Such compounds are identical to those disclosed herein, but for the fact that one or more atoms are replaced by an atom having an atomic mass or mass number different from the atomic mass or mass number usually found in nature. Examples of isotopes suitable for inclusion in the compounds of the invention include isotopes of hydrogen such as ²H and ³H, carbon such as ¹¹C, ¹³C and ¹⁴C, nitrogen such as ¹³N and ¹⁵N, oxygen such as ¹⁵O, ¹⁷O and ¹⁸O, phosphorus such as ³²P, sulfur such as ³⁵S, fluorine such as ¹⁸F, iodine such as ¹²³I and ¹²⁵I, and chlorine such as ³⁶Cl. Certain isotopically-labelled compounds of Formula I, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. An embodiment of the present invention includes compounds that are substituted with a positron emitting isotope. An embodiment of the present invention includes compounds that are substituted with a ¹¹C isotope. An embodiment of the present invention includes compounds that are substituted with an ¹⁸F isotope. In the compounds of the invention, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds of the invention can be prepared without undue experimentation by conventional techniques well known to those skilled in the art or by processes analogous to those described in the schemes and examples herein using appropriate isotopically-enriched reagents and/or intermediates.

When any variable (e.g., R⁴, R⁵ etc.) occurs more than one time in any substituent or in Formula I, its definition on each occurrence is independent of its definition at every other occurrence unless otherwise specified at the point of definition. One of ordinary skill in the art will recognize that choice of combinations of the various substituents defined in a structural representation, i.e. R², R⁵, etc., are to be chosen in conformity with well-known principles of chemical structure connectivity and stability, and combinations of substituents and/or variables are permissible only if such combinations result in stable compounds.

A “stable” compound is a compound which can be prepared and isolated and whose structure and properties remain or can be caused to remain essentially unchanged for a period of time sufficient to allow use of the compound for the purposes described herein (e.g., therapeutic administration to a subject). The compounds of the present invention are limited to stable compounds embraced by Formula I.

Those skilled in the art will recognize those instances in which the compounds of the invention may form salts. In such instances, another embodiment provides pharmaceutically acceptable salts of the compounds of the invention. Thus, reference to a compound of the invention herein is understood to include reference to salts thereof, unless otherwise indicated. The term “pharmaceutically acceptable salts” refers to salts prepared from pharmaceutically acceptable non-toxic bases or acids including inorganic or organic bases and inorganic or organic acids. In addition, when a compound of the invention contains both a basic moiety, such as, but not limited to a pyridine or imidazole, and an acidic moiety, such as, but not limited to a carboxylic acid, zwitterions (“inner salts”) may be formed and are included within the present invention. Salts derived from inorganic bases include aluminum, ammonium, calcium, copper, ferric, ferrous, lithium, magnesium, manganic salts, manganous, potassium, sodium, zinc, and the like. Particular embodiments include the ammonium, calcium, magnesium, potassium, and sodium salts. Salts in the solid form may exist in more than one crystal structure, and may also be in the form of hydrates or solvates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particular embodiments include the citric, hydrobromic, hydrochloric, maleic, phosphoric, sulfuric, fumaric, and tartaric acids. It will be understood that, as used herein, references to the compounds of Formula I are meant to also include the pharmaceutically acceptable salts. Salts of the compounds of the invention may be formed by methods known to those of ordinary skill in the art, for example, by reacting a compound of the invention with an amount of acid or base, such as an equivalent amount, in a medium such as one in which the salt precipitates or in an aqueous medium followed by lyophilization.

Exemplifying the invention is the use of the compounds disclosed in the Examples and herein. Specific compounds within the present invention include a compound which is selected from the compounds disclosed in the following Examples and pharmaceutically acceptable salts thereof and individual enantiomers or diastereomers thereof.

The present invention is also directed to the use of the compounds disclosed herein as agonists of orexin receptor activity. The subject compounds and pharmaceutically acceptable salts thereof are useful in a method of agonizing orexin receptor activity in a subject such as a mammal comprising the administration of an amount of the compound. In addition to primates, especially humans, a variety of other mammals may be administered with a compound of the present invention. The present invention is directed to a compound of the present invention or a pharmaceutically acceptable salt thereof that could be useful in therapy. The present invention may further be directed to a use of a compound of the present invention or a pharmaceutically acceptable salt thereof for the manufacture of a medicament for agonizing orexin receptor activity or treating the disorders and diseases noted herein in humans and animals.

A subject administered with a compound of the present invention, or a pharmaceutically acceptable salt thereof, is generally a mammal, such as a human being, male or female. The amount of compound administered to the subject is an amount sufficient to agonize the orexin receptor in the subject. In an embodiment, the amount of compound can be an “effective amount”, wherein the subject compound is administered in an amount that will elicit the biological or medical response of a tissue, system, animal or human that is being sought by the researcher, veterinarian, medical doctor or other clinician. An effective amount does not necessarily include considerations of toxicity and safety related to the administration of the compound. It is recognized that one skilled in the art may affect neurological and psychiatric disorders associated with orexin receptor activation by treating a subject presently afflicted with the disorders, or by prophylactically treating a subject likely to be afflicted with the disorders, with an effective amount of a compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy of the mentioned conditions, particularly in a subject that is predisposed to such disease or disorder. The terms “administration of” and or “administering a” compound should be understood to mean providing a compound of the invention or a prodrug of a compound of the invention to the subject.

The term “composition” as used herein is intended to encompass a product comprising the specified ingredients in the specified amounts, as well as any product which results, directly or indirectly, from combination of the specified ingredients in the specified amounts. Such term is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The utility of the compounds in accordance with the present invention as orexin receptor OX1R and/or OX2R agonists may be readily determined without undue experimentation by methodology well known in the art. Both the OX1R and/or OX2R G-coupled protein receptors (GPCRs) couple through the Gαq signaling pathway, which ultimately promotes calcium mobilization via inositol triphosphate (IP3) production. The half-life of IP-3 is relatively short, being rapidly metabolized to inositol monophosphate (IP-1), which can be readily detected using a commercially available assay kit (IP-One; Cisbio; cat #621PAPEC) coupled with a cell line expressing the target receptor(s) of interest. The utility of the compounds in accordance with the present invention as orexin receptor OX1R and/or OX2R agonists may be determined utilizing this assay.

In a typical experiment, the OX1 and OX2 receptor agonist activity is determined in accordance with the following general experimental method. Chinese hamster ovary (CHO) cells expressing human OX1R and/or the human OX2R were grown in Iscove's modified DMEM containing glutaMAX™, 1% G418, 100 U/mL penicillin, 100 μg/mL streptomycin and 10% heat-inactivated qualified fetal bovine serum (FBS). The OX2R cells were seeded at 10,000 cells/well/50 μL and the OX1R cells were seeded at 20,000 cells/well/50 μL into 384-well white tissue culture plates (Greiner; cat #781080). All cell/media reagents were from GIBCO-Invitrogen Corp. The seeded cell plate(s) were incubated at 37° C. with 5% CO₂ and 85% humidity for 20-24 hours. On the day of the assay, assay-ready compound plates were prepared using an acoustic liquid handler (ECHO; Labcyte), which dispensed sufficient volume of test compound stock (10 mM in DMSO) or 100% DMSO to prepare 10 point, ½-log dilutions in a final volume of 202.5 nL/well in all test wells of a 384-well diamond plate (Labcyte). Following completion of assay-ready plates, importantly, the next three steps were performed with minimal delay: 1) 20 μl of 1× stimulation buffer was added to the compound plate using a Multidrop Combi (small cassette, Thermo Fisher Scientific cat #24073290); 2) culture medium was removed from the cell plate using the Bluewasher plate washer (gentle spin; BlueCatBio); 3) 14 μl of compound/stimulation buffer mixture was added to the cell plate using a Bravo liquid handler (Agilent) prior to incubating cell plates at 37° C. with 5% CO₂ and 85% humidity for 1 or 2 hours (OX1R and OX2R, respectively). During this incubation, IP-one detection reagents were prepared (38:1:1 lysis buffer:D2:AB-cryptate reagents). Six μL of mixed detection reagents were added to the cell plate using a Multidrop Combi (small cassette, Thermo Fisher Scientific cat #24073290) and incubated 60 minutes at room temperature in the dark. Fluorescence signal was detected using an Envision plate reader (Perkin Elmer) [LANCE/DELFIA Dual Enh (Em: APC 665; Ex: Cy5 620)].

For each compound, data were fit to a four parameter logistic fit (ActivityBase software) and the EC₅₀ was reported as the inflection point of the resulting curve. Percent effect for each test compound was determined as the percentage of sample raw value/mean max effect, where the mean max effect was derived from the mean raw value of 32 control wells per assay plate (using Orexin A (cat #003-30) at 1 μM for human OX1R and a reference compound at 1 uM with 100% activity previously established by comparison to Orexin A for human OX2R). The intrinsic orexin receptor agonist activity of a compound which may be used in the present invention may be determined by these assays.

All of the final compounds of the following examples had activity in agonizing the human orexin-2 receptor in the aforementioned IPOne assay with an EC₅₀ of about 0.01 nM to 5000 nM. Additional data is provided in the following Examples. Such a result is indicative of the intrinsic activity of the compounds in use as agonists of orexin-1 receptor and/or the orexin-2 receptor. In general, one of ordinary skill in the art would appreciate that a substance is considered to effectively agonize the orexin receptor if it has an EC₅₀ in the IPOne assay of less than about 50 μM, or more specifically less than about 1000 nM.

The orexin receptors have been implicated in a wide range of biological functions. This has suggested a potential role for these receptors in a variety of disease processes in humans or other species. The compounds of the present invention could therefore potentially have utility in treating, preventing, ameliorating, controlling or reducing the risk of a variety of disorders associated with orexin receptors, including one or more of the following conditions or diseases: narcolepsy, narcolepsy syndrome accompanied by narcolepsy-like symptoms, cataplexy in narcolepsy, excessive daytime sleepiness (EDS) in narcolepsy, hypersomnia, idiopathic hypersomnia, repeatability hypersomnia, intrinsic hypersomnia, hypersomnia accompanied by daytime hypersomnia, interrupted sleep, sleep apnea, wakefulness, nocturnal myoclonus, disturbances of consciousness, such as coma, REM sleep interruptions, jet-lag, excessive daytime sleepiness, shift workers' sleep disturbances, dyssomnias, sleep disorders, sleep disturbances, hypersomnia associated with depression, emotional/mood disorders, Alzheimer's disease or cognitive impairment, Parkinson's disease, Guillain-Barre syndrome, Kleine Levin syndrome, and sleep disorders which accompany aging; Alzheimer's sundowning; conditions associated with circadian rhythmicity as well as mental and physical disorders associated with travel across time zones and with rotating shift-work schedules; fibromyalgia; cardiac failure; diseases related to bone loss; sepsis; syndromes which are manifested by non-restorative sleep and muscle pain or sleep apnea which is associated with respiratory disturbances during sleep; conditions which result from a diminished quality of sleep; and other diseases related to general orexin system dysfunction.

Thus, in certain embodiments the present invention may provide methods for: treating or controlling narcolepsy, narcolepsy syndrome accompanied by narcolepsy-like symptoms, cataplexy in narcolepsy, excessive daytime sleepiness (EDS) in narcolepsy, hypersomnia, idiopathic hypersomnia, repeatability hypersomnia, intrinsic hypersomnia, hypersomnia accompanied by daytime hypersomnia, interrupted sleep, sleep apnea, disturbances of consciousness, REM sleep interruptions, jet-lag, shift workers' sleep disturbances, dyssomnias, night terror, insomnias associated with depression, emotional/mood disorders, Alzheimer's disease or cognitive impairment; treating or controlling sleep disturbances associated with diseases such as neurological disorders including neuropathic pain and restless leg syndrome; treating or controlling addiction disorders; treating or controlling psychoactive substance use and abuse; enhancing cognition; increasing memory retention; treating or controlling obesity; treating or controlling diabetes and appetite, taste, eating, or drinking disorders; treating or controlling insulin resistance syndrome; treating or controlling hypothalamic diseases; treating or controlling depression; treating, controlling, ameliorating or reducing the risk of epilepsy, including absence epilepsy; treating or controlling pain, including neuropathic pain; treating or controlling Parkinson's disease; treating or controlling Guillain-Barre syndrome; treating or controlling Klein Levin syndrome; treating or controlling psychosis; treating or controlling dysthymic, mood, psychotic and anxiety disorders; treating side effects or complications due to anesthesia; reversal of anesthesia; reversal of anesthesia following surgery; treating or controlling depression, including major depression and major depression disorder; treating or controlling bipolar disorder; or treating, controlling, ameliorating or reducing the risk of schizophrenia, in a mammalian subject which comprises administering to the subject a compound of the present invention.

The compounds of the present invention may also potentially have utility in treating, preventing, ameliorating, controlling or reducing the risk of a variety of other disorders associated with orexin receptors, including one or more of the following conditions or diseases including enhancing sleep quality, improving sleep quality, increasing sleep efficiency, augmenting sleep maintenance; increasing the value which is calculated from the time that a subject sleeps divided by the time that a subject is attempting to sleep; improving sleep initiation; decreasing sleep latency or onset (the time it takes to fall asleep); decreasing difficulties in falling asleep; increasing sleep continuity; decreasing the number of awakenings during sleep; decreasing intermittent wakings during sleep; decreasing nocturnal arousals; decreasing the time spent awake following the initial onset of sleep; increasing the total amount of sleep; reducing the fragmentation of sleep; altering the timing, frequency or duration of REM sleep bouts; altering the timing, frequency or duration of slow wave (i.e. stages 3 or 4) sleep bouts; increasing the amount and percentage of stage 2 sleep; promoting slow wave sleep; enhancing EEG-delta activity during sleep; decreasing nocturnal arousals, especially early morning awakenings; increasing daytime alertness, reducing daytime drowsiness; treating or reducing excessive daytime sleepiness; increasing satisfaction with the intensity of sleep; increasing sleep maintenance; idiopathic insomnia; sleep problems; insomnia; night terror, insomnias associated with depression, emotional/mood disorders, Alzheimer's disease or cognitive impairment, as well as sleep walking and enuresis, and sleep disorders which accompany aging; Alzheimer's sundowning; conditions associated with circadian rhythmicity as well as mental and physical disorders associated with travel across time zones and with rotating shift-work schedules, conditions due to drugs which cause reductions in REM sleep as a side effect; fibromyalgia; syndromes which are manifested by non-restorative sleep and muscle pain or sleep apnea which is associated with respiratory disturbances during sleep; conditions which result from a diminished quality of sleep; increasing learning; augmenting memory; increasing retention of memory; eating disorders associated with excessive food intake and complications associated therewith, compulsive eating disorders, obesity (due to any cause, whether genetic or environmental), obesity-related disorders overeating, anorexia, bulimia, cachexia, dysregulated appetite control, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, lung disease, abnormal heart rhythms and arrythmias, myocardial infarction, congestive heart failure, coronary heart disease, acute and congestive heart failure; hypotension; hypertension; urinary retention; osteoporosis; angina pectoris; myocardinal infarction; ischemic or haemorrhagic stroke; subarachnoid haemorrhage; ulcers; allergies; benign prostatic hypertrophy; chronic renal failure; renal disease; impaired glucose tolerance; sudden death, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia, metabolic syndrome, also known as syndrome X, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, intestinal motility dyskinesias, obesity-related gastro-esophageal reflux, hypothalmic diseases, hypophysis diseases, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, hyperuricemia, lower back pain, gallbladder disease, gout, kidney cancer, increased anesthetic risk, reducing the risk of secondary outcomes of obesity, such as reducing the risk of left ventricular hypertrophy; diseases or disorders where abnormal oscillatory activity occurs in the brain, including depression, migraine, neuropathic pain, Parkinson's disease, psychosis and schizophrenia, as well as diseases or disorders where there is abnormal coupling of activity, particularly through the thalamus; enhancing cognitive function, including cognitive dysfunctions that comprise deficits in all types of attention, learning and memory functions occurring transiently or chronically in the normal, healthy, young, adult or aging population, and also occurring transiently or chronically in psychiatric, neurologic, cardiovascular and immune disorders; treating or controlling Guillain-Barre syndrome; treating or controlling Klein Levin syndrome; treating or controlling psychosis; treating or controlling dysthymic, mood, psychotic and anxiety disorders; treating complications due to anesthesia; enhancing memory; increasing memory retention; increasing immune response; increasing immune function; hot flashes; night sweats; extending life span; schizophrenia; muscle-related disorders that are controlled by the excitation/relaxation rhythms imposed by the neural system such as cardiac rhythm and other disorders of the cardiovascular system; conditions related to proliferation of cells such as vasodilation or vasorestriction and blood pressure; cancer; cardiac arrhythmia; hypertension; congestive heart failure; conditions of the genital/urinary system; disorders of sexual function and fertility; adequacy of renal function: responsivity to anesthetics; mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder, mood disorders due to a general medical condition, and substance-induced mood disorders; affective neurosis; depressive neurosis; anxiety neurosis; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; acute neurological and psychiatric disorders such as cerebral deficits subsequent to cardiac bypass surgery and grafting, stroke, ischemic stroke, cerebral ischemia, spinal cord trauma, head trauma, perinatal hypoxia, cardiac arrest, hypoglycemic neuronal damage; Huntington's Chorea; Huntington's disease and Tourette syndrome; Cushing's syndrome/disease; basophile adenoma; prolactinoma; hyperprolactinemia; hypophysis tumor/adenoma; hypothalamic diseases; inflammatory bowel disease; gastric diskinesia; gastric ulcers; Froehlich's syndrome; adrenohypophysis disease; hypophysis disease; adrenohypophysis hypofunction; adrenohypophysis hyperfunction; hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth deficiency; dwarfism; gigantism; acromegaly; amyotrophic lateral sclerosis; multiple sclerosis; ocular damage; retinopathy; cognitive disorders, idiopathic and drug-induced Parkinson's disease; muscular spasms and disorders associated with muscular spasticity including tremors, epilepsy, convulsions, seizure disorders, absence seizures, complex partial and generalized seizures; Lennox-Gastaut syndrome; cognitive disorders including dementia (associated with Alzheimer's disease, ischemia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age related cognitive decline; schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced psychotic disorder; dissociative disorders including multiple personality syndromes and psychogenic amnesias; substance-related disorders, substance use, substance abuse, substance seeking, substance reinstatement, all types of psychological and physical addictions and addictive behaviors, reward-related behaviors (including substance-induced delirium, persisting dementia, persisting amnestic disorder, psychotic disorder or anxiety disorder; tolerance, addictive feeding, addictive feeding behaviors, binge/purge feeding behaviors, dependence, withdrawal or relapse from substances including alcohol, amphetamines, cannabis, cocaine, hallucinogens, inhalants, morphine, nicotine, opioids, phencyclidine, sedatives, hypnotics or anxiolytics); appetite, taste, eating or drinking disorders; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), chronic fatigue syndrome, fatigue, including Parkinson's fatigue, multiple sclerosis fatigue, fatigue caused by a sleep disorder or a circadian rhythm disorder, medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, and dyskinesias [including tremor (such as rest tremor, essential tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), restless leg syndrome and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia); neurodegenerative disorders including nosological entities such as disinhibition-dementia-parkinsonism-amyotrophy complex; pallido-ponto-nigral degeneration; epilepsy; seizure disorders; attention deficit/hyperactivity disorder (ADHD); conduct disorder; migraine (including migraine headache); headache; hyperalgesia; pain; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; pain related to infection e.g. HIV, post-chemotherapy pain; post-stroke pain; post-operative pain; neuralgia; emesis, nausea, vomiting; gastric dyskinesia; gastric ulcers; Kallman's syndrome (anosmia); asthma; cancer; conditions associated with visceral pain such as irritable bowel syndrome, and angina; eating disorders; urinary incontinence; substance tolerance, substance withdrawal (including, substances such as opiates, nicotine, tobacco products, alcohol, benzodiazepines, cocaine, sedatives, hypnotics, etc.); psychosis; schizophrenia; anxiety (including generalized anxiety disorder, panic disorder, and obsessive compulsive disorder); mood disorders (including depression, mania, bipolar disorders); trigeminal neuralgia; hearing loss; tinnitus; neuronal damage including ocular damage; retinopathy; macular degeneration of the eye; emesis; brain edema; pain, including acute and chronic pain states, severe pain, intractable pain, inflammatory pain, neuropathic pain, post-traumatic pain, bone and joint pain (osteoarthritis), repetitive motion pain, dental pain, cancer pain, myofascial pain (muscular injury, fibromyalgia), perioperative pain (general surgery, gynecological), chronic pain, neuropathic pain, post-traumatic pain, trigeminal neuralgia, migraine and migraine headache and other diseases related to general orexin system dysfunction.

The subject compounds could further be of potential use in a method for the prevention, treatment, control, amelioration, or reduction of risk of the diseases, disorders and conditions noted herein. The dosage of active ingredient in the compositions of this invention may be varied, however, it is necessary that the amount of the active ingredient be such that a suitable dosage form is obtained. The active ingredient may be administered to subjects (animals and human) in need of such treatment in dosages that will provide optimal pharmaceutical efficacy. The selected dosage depends upon the desired therapeutic effect, on the route of administration, and on the duration of the treatment. The dose will vary from subject to subject depending upon the nature and severity of disease, the subject's weight, special diets then being followed by a subject, concurrent medication, and other factors which those skilled in the art will recognize.

Generally, dosage levels of between 0.0001 to 100 mg/kg. of body weight daily are administered to the subject, e.g., humans, adolescent humans and elderly humans, to obtain effective agonism of orexin receptors. The dosage range will generally be about 0.5 mg to 10.0 g. per subject per day which may be administered in single or multiple doses. In one embodiment, the dosage range will be about 0.5 mg to 500 mg per subject per day; in another embodiment about 0.5 mg to 200 mg per subject per day; and in yet another embodiment about 5 mg to 50 mg per subject per day. Pharmaceutical compositions of the present invention may be provided in a solid dosage formulation such as comprising about 0.5 mg to 500 mg active ingredient, or comprising about 1 mg to 250 mg active ingredient. The pharmaceutical composition may be provided in a solid dosage formulation comprising about 1 mg, 5 mg, 10 mg, 25 mg, 30 mg, 50 mg, 80 mg, 100 mg, 200 mg or 250 mg active ingredient. For oral administration, the compositions may be provided in the form of tablets containing 1.0 to 1000 milligrams of the active ingredient, such as 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the subject to be treated. The compounds may be administered on a regimen of 1 to 4 times per day, such as once or twice per day. The compounds may be administered once or multiple times during the day. The compounds may be administered upon awakening or otherwise in the morning, or during waking hours. For example, the compounds may be administered about 1 hour after awakening, about 30 minutes after awakening or immediately after awakening.

The compounds of the present invention may be used in combination with one or more other drugs in the treatment, prevention, control, amelioration, or reduction of risk of diseases or conditions for which compounds of the present invention or the other drugs may have utility, where the combination of the drugs together are safer or more effective than either drug alone. Such other drug(s) may be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and the compound of the present invention is contemplated. However, the combination therapy may also include therapies in which the compound of the present invention and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of the present invention. The above combinations include combinations of a compound of the present invention not only with one other active compound, but also with two or more other active compounds.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, such as about 200:1 to about 1:200. Combinations of a compound of the present invention and other active ingredients will generally also be within the aforementioned range, but in each case, an effective dose of each active ingredient should be used. In such combinations the compound of the present invention and other active agents may be administered separately or in conjunction. In addition, the administration of one element may be prior to, concurrent to, or subsequent to the administration of other agent(s).

The compounds of the present invention may be administered in combination with compounds which are known in the art to be useful for treating or controlling narcolepsy, including e.g., methylphenidate, amphetamine, pemoline, phenelzine, protriptyline, gamma-hydroxybutyric acid, sodium oxybate, or other oxybate salts, modafinil, armodafinil, caffeine, and salts thereof, and combinations thereof, and the like.

The compounds of the present invention may be administered in combination with compounds which are known in the art to be useful for preventing and treating sleep disorders and sleep disturbances, including e.g., sedatives, hypnotics, anxiolytics, antipsychotics, antianxiety agents, antihistamines, benzodiazepines, barbiturates, cyclopyrrolones, GABA agonists, 5HT-2 antagonists including 5HT-2A antagonists and 5HT-2A/2C antagonists, histamine antagonists including histamine H3 antagonists, histamine H3 inverse agonists, imidazopyridines, minor tranquilizers, melatonin agonists and antagonists, melatonergic agents, orexin antagonists, other orexin agonists, prokineticin agonists and antagonists, pyrazolopyrimidines, T-type calcium channel antagonists, triazolopyridines, and the like, such as: adinazolam, allobarbital, alonimid, alprazolam, amitriptyline, amobarbital, amoxapine, armodafinil, APD-125, bentazepam, benzoctamine, brotizolam, bupropion, busprione, butabarbital, butalbital, capromorelin, capuride, carbocloral, chloral betaine, chloral hydrate, chlordiazepoxide, clomipramine, clonazepam, cloperidone, clorazepate, clorethate, clozapine, conazepam, cyprazepam, desipramine, dexclamol, diazepam, dichloralphenazone, divalproex, diphenhydramine, doxepin, EMD-281014, eplivanserin, estazolam, eszopiclone, ethchlorynol, etomidate, fenobam, flunitrazepam, flurazepam, fluvoxamine, fluoxetine, fosazepam, gaboxadol, glutethimide, halazepam, hydroxyzine, ibutamoren, imipramine, indiplon, lithium, lorazepam, lormetazepam, LY-156735, maprotiline, MDL-100907, mecloqualone, melatonin, mephobarbital, meprobamate, methaqualone, methyprylon, midaflur, midazolam, modafnil, nefazodone, NGD-2-73, nisobamate, nitrazepam, nortriptyline, omortriptyline, oxazepam, paraldehyde, paroxetine, pentobarbital, perlapine, perphenazine, phenelzine, phenobarbital, prazepam, promethazine, propofol, protriptyline, quazepam, ramelteon, reclazepam, roletamide, secobarbital, sertraline, suproclone, TAK-375, temazepam, thioridazine, tiagabine, tracazolate, tranylcypromaine, trazodone, triazolam, trepipam, tricetamide, triclofos, trifluoperazine, trimetozine, trimipramine, uldazepam, venlafaxine, zaleplon, zolazepam, zopiclone, zolpidem, and salts thereof, and combinations thereof, and the like, or the compound of the present invention may be administered in conjunction with the use of physical methods such as with light therapy or electrical stimulation.

In another embodiment, the subject compound may be employed in combination with other compounds which are known in the art, either administered separately or in the same pharmaceutical compositions, including, but are not limited to: insulin sensitizers including (i) PPARγ antagonists such as glitazones (e.g. ciglitazone; darglitazone; englitazone; isaglitazone (MCC-555); pioglitazone; rosiglitazone; troglitazone; tularik; BRL49653; CLX-0921; 5-BTZD), GW-0207, LG-100641, and LY-300512, and the like); (iii) biguanides such as metformin and phenformin; (b) insulin or insulin mimetics, such as biota, LP-100, novarapid, insulin detemir, insulin lispro, insulin glargine, insulin zinc suspension (lente and ultralente); Lys-Pro insulin, GLP-1 (73-7) (insulintropin); and GLP-1 (7-36)-NH₂); (c) sulfonylureas, such as acetohexamide; chlorpropamide; diabinese; glibenclamide; glipizide; glyburide; glimepiride; gliclazide; glipentide; gliquidone; glisolamide; tolazamide; and tolbutamide; (d) α-glucosidase inhibitors, such as acarbose, adiposine; camiglibose; emiglitate; miglitol; voglibose; pradimicin-Q; salbostatin; CKD-711; MDL-25,637; MDL-73,945; and MOR 14, and the like; (e) cholesterol lowering agents such as (i) HMG-CoA reductase inhibitors (atorvastatin, itavastatin, fluvastatin, lovastatin, pravastatin, rivastatin, rosuvastatin, simvastatin, and other statins), (ii) bile acid absorbers/sequestrants, such as cholestyramine, colestipol, dialkylaminoalkyl derivatives of a cross-linked dextran; Colestid®; LoCholest®, and the like, (ii) nicotinyl alcohol, nicotinic acid or a salt thereof, (iii) proliferator-activator receptor α agonists such as fenofibric acid derivatives (gemfibrozil, clofibrate, fenofibrate and benzafibrate), (iv) inhibitors of cholesterol absorption such as stanol esters, beta-sitosterol, sterol glycosides such as tiqueside; and azetidinones such as ezetimibe, and the like, and (acyl CoA:cholesterol acyltransferase (ACAT)) inhibitors such as avasimibe, and melinamide, (v) anti-oxidants, such as probucol, (vi) vitamin E, and (vii) thyromimetics; (f) PPARα agonists such as beclofibrate, benzafibrate, ciprofibrate, clofibrate, etofibrate, fenofibrate, and gemfibrozil; and other fibric acid derivatives, such as Atromid®, Lopid® and Tricor®, and the like, and PPARα agonists as described in WO 97/36579; (g) PPARδ agonists, such as those disclosed in WO 97/28149; (h) PPAR α/δ agonists, such as muraglitazar, and the compounds disclosed in U.S. Pat. No. 6,414,002; (i) anti-obesity agents, such as (l) growth hormone secretagogues, growth hormone secretagogue receptor agonists/antagonists, such as NN703, hexarelin, MK-0677, SM-130686, CP-424,391, L-692,429, and L-163,255, and such as those disclosed in U.S. Pat. Nos. 5,536,716, and 6,358,951, U.S. Patent Application Nos. 2002/049196 and 2002/022637, and PCT Application Nos. WO 01/56592 and WO 02/32888; (2) protein tyrosine phosphatase-1B (PTP-1B) inhibitors; (3) cannabinoid receptor ligands, such as cannabinoid CB₁ receptor antagonists or inverse agonists, such as rimonabant, taranabant, AMT-251, and SR-14778 and SR 141716A (Sanofi Synthelabo), SLV-319 (Solvay), BAY 65-2520 (Bayer) and those disclosed in U.S. Pat. Nos. 5,532,237, 4,973,587, 5,013,837, 5,081,122, 5,112,820, 5,292,736, 5,624,941, 6,028,084, PCT Application Nos. WO 96/33159, WO 98/33765, WO 98/43636, WO 98/43635, WO 01/09120, WO 98/31227, WO 98/41519, WO 98/37061, WO 00/10967, WO 00/10968, WO97/29079, WO99/02499, WO 01/58869, WO 01/64632, WO 01/64633, WO 01/64634, WO 02/076949, WO 03/007887, WO 04/048317, and WO 05/000809; (4) anti-obesity serotonergic agents, such as fenfluramine, dexfenfluramine, phentermine, and sibutramine; (5) β3-adrenoreceptor agonists, such as AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, SR 59119A; (6) pancreatic lipase inhibitors, such as orlistat (Xenical®), Triton WR1339, RHC80267, lipstatin, tetrahydrolipstatin, teasaponin, diethylumbelliferyl phosphate, and those disclosed in PCT Application No. WO 01/77094; (7) neuropeptide Y1 antagonists, such as BIBP3226, J-115814, BIBO 3304, LY-357897, CP-671906, GI-264879A, and those disclosed in U.S. Pat. No. 6,001,836, and PCT Patent Publication Nos. WO 96/14307, WO 01/23387, WO 99/51600, WO 01/85690, WO 01/85098, WO 01/85173, and WO 01/89528; (8) neuropeptide Y5 antagonists, such as GW-569180A, GW-594884A, GW-587081X, GW-548118X, FR226928, FR 240662, FR252384, 1229U91, GI-264879A, CGP71683A, LY-377897, PD-160170, SR-120562A, SR-120819A and JCF-104, and those disclosed in U.S. Pat. Nos. 6,057,335; 6,043,246; 6,140,354; 6,166,038; 6,180,653; 6,191,160; 6,313,298; 6,335,345; 6,337,332; 6,326,375; 6,329,395; 6,340,683; 6,388,077; 6,462,053; 6,649,624; and 6,723,847, European Patent Nos. EP-01010691, and EP-01044970; and PCT International Patent Publication Nos. WO 97/19682, WO 97/20820, WO 97/20821, WO 97/20822, WO 97/20823, WO 98/24768; WO 98/25907; WO 98/25908; WO 98/27063, WO 98/47505; WO 98/40356; WO 99/15516; WO 99/27965; WO 00/64880, WO 00/68197, WO 00/69849, WO 01/09120, WO 01/14376; WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/22592, WO 0248152, and WO 02/49648; WO 02/094825; WO 03/014083; WO 03/10191; WO 03/092889; WO 04/002986; and WO 04/031175; (9) melanin-concentrating hormone (MCH) receptor antagonists, such as those disclosed in WO 01/21577 and WO 01/21169; (10) melanin-concentrating hormone 1 receptor (MCH1R) antagonists, such as T-226296 (Takeda), and those disclosed in PCT Patent Application Nos. WO 01/82925, WO 01/87834, WO 02/051809, WO 02/06245, WO 02/076929, WO 02/076947, WO 02/04433, WO 02/51809, WO 02/083134, WO 02/094799, WO 03/004027; (11) melanin-concentrating hormone 2 receptor (MCH2R) agonist/antagonists; (12) orexin receptor antagonists, such as SB-334867-A, and those disclosed in patent publications herein; (13) serotonin reuptake inhibitors such as fluoxetine, paroxetine, and sertraline; (14) melanocortin agonists, such as Melanotan II; (15) Mc4r (melanocortin 4 receptor) agonists, such as CHIR86036 (Chiron), ME-10142, and ME-10145 (Melacure), CHIR86036 (Chiron); PT-141, and PT-14 (Palatin); (16) 5HT-2 agonists; (17) 5HT2C (serotonin receptor 2C) agonists, such as BVT933, DPCA37215, WAY161503, R-1065, and those disclosed in U.S. Pat. No. 3,914,250, and PCT Application Nos. WO 02/36596, WO 02/48124, WO 02/10169, WO 01/66548, WO 02/44152, WO 02/51844, WO 02/40456, and WO 02/40457; (18) galanin antagonists; (19) CCK agonists; (20) CCK-A (cholecystokinin-A) agonists, such as AR-R 15849, GI 181771, JMV-180, A-71378, A-71623 and SR14613, and those described in U.S. Pat. No. 5,739,106; (21) GLP-1 agonists; (22) corticotropin-releasing hormone agonists; (23) histamine receptor-3 (H3) modulators; (24) histamine receptor-3 (H3) antagonists/inverse agonists, such as thioperamide, 3-(1H-imidazol-4-yl)propyl N-(4-pentenyl)carbamate, clobenpropit, iodophenpropit, imoproxifan, GT2394 (Gliatech), and O-[3-(1H-imidazol-4-yl)propanol]-carbamates; (25) β-hydroxy steroid dehydrogenase-1 inhibitors (3-HSD-1); (26) PDE (phosphodiesterase) inhibitors, such as theophylline, pentoxifylline, zaprinast, sildenafil, amrinone, milrinone, cilostamide, rolipram, and cilomilast; (27) phosphodiesterase-3B (PDE3B) inhibitors; (28) NE (norepinephrine) transport inhibitors, such as GW 320659, despiramine, talsupram, and nomifensine; (29) ghrelin receptor antagonists, such as those disclosed in PCT Application Nos. WO 01/87335, and WO 02/08250; (30) leptin, including recombinant human leptin (PEG-OB, Hoffman La Roche) and recombinant methionyl human leptin (Amgen); (31) leptin derivatives; (32) BRS3 (bombesin receptor subtype 3) agonists such as [D-Phe6,beta-Ala11,Phe13,Nle14]Bn(6-14) and [D-Phe6,Phe13]Bn(6-13)propylamide, and those compounds disclosed in Pept. Sci. 2002 August; 8(8): 461-75); (33) CNTF (Ciliary neurotrophic factors), such as GI-181771 (Glaxo-SmithKline), SR146131 (Sanofi Synthelabo), butabindide, PD170,292, and PD 149164 (Pfizer); (34) CNTF derivatives, such as axokine (Regeneron); (35) monoamine reuptake inhibitors, such as sibutramine; (36) UCP-1 (uncoupling protein-1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), retinoic acid; (37) thyroid hormone β agonists, such as KB-2611 (KaroBioBMS); (38) FAS (fatty acid synthase) inhibitors, such as Cerulenin and C75; (39) DGAT1 (diacylglycerol acyltransferase 1) inhibitors; (40) DGAT2 (diacylglycerol acyltransferase 2) inhibitors; (41) ACC2 (acetyl-CoA carboxylase-2) inhibitors; (42) glucocorticoid antagonists; (43) acyl-estrogens, such as oleoyl-estrone, disclosed in del Mar-Grasa, M. et al., Obesity Research, 9:202-9 (2001); (44) dipeptidyl peptidase IV (DP-IV) inhibitors, such as isoleucine thiazolidide, valine pyrrolidine, NVP-DPP728, LAF237, P93/01, TSL 225, TMC-2A/2B/2C, FE 999011, P9310/K364, VIP 0177, SDZ 274-444, sitagliptin; and the compounds disclosed in U.S. Pat. No. 6,699,871, WO 03/004498; WO 03/004496; EP 1 258 476; WO 02/083128; WO 02/062764: WO 03/000250; WO 03/002530; WO 03/002531; WO 03/002553; WO 03/002593; WO 03/000180; and WO 03/000181; (46) dicarboxylate transporter inhibitors; (47) glucose transporter inhibitors; (48) phosphate transporter inhibitors; (49) Metformin (Glucophage®); (50) Topiramate (Topimax®); (50) peptide YY, PYY 3-36, peptide YY analogs, derivatives, and fragments such as BIM-43073D, BIM-43004C (Olitvak, D. A. et al., Dig. Dis. Sci. 44(3):643-48 (1999)); (51) Neuropeptide Y2 (NPY2) receptor agonists such NPY3-36, N acetyl [Leu(28,31)] NPY 24-36, TASP-V, and cyclo-(28/32)-Ac-[Lys28-Glu32]-(25-36)-pNPY; (52) Neuropeptide Y4 (NPY4) agonists such as pancreatic peptide (PP), and other Y4 agonists such as 1229U91; (54) cyclooxygenase-2 inhibitors such as etoricoxib, celecoxib, valdecoxib, parecoxib, lumiracoxib, BMS347070, tiracoxib or JTE522, ABT963, CS502 and GW406381; (55) Neuropeptide Y1 (NPY1) antagonists such as BIBP3226, J-115814, BIBO 3304, LY-357897, CP-671906, GI-264879A; (56) Opioid antagonists such as nalmefene (Revex®), 3-methoxynaltrexone, naloxone, naltrexone; (57) 110 HSD-1 (11-beta hydroxy steroid dehydrogenase type 1) inhibitors such as BVT 3498, BVT 2733, and those disclosed in WO 01/90091, WO 01/90090, WO 01/90092, U.S. Pat. No. 6,730,690 and US 2004-0133011; (58) aminorex; (59) amphechloral; (60) amphetamine; (61) benzphetamine; (62) chlorphentermine; (63) clobenzorex; (64) cloforex; (65) clominorex; (66) clortermine; (67) cyclexedrine; (68) dextroamphetamine; (69) diphemethoxidine, (70) N-ethylamphetamine; (71) fenbutrazate; (72) fenisorex; (73) fenproporex; (74) fludorex; (75) fluminorex; (76) furfurylmethylamphetamine; (77) levamfetamine; (78) levophacetoperane; (79) mefenorex; (80) metamfepramone; (81) methamphetamine; (82) norpseudoephedrine; (83) pentorex; (84) phendimetrazine; (85) phenmetrazine; (86) picilorex; (87) phytopharm 57; and (88) zonisamide., (89) neuromedin U and analogs or derivatives thereof, (90) oxyntomodulin and analogs or derivatives thereof, and (91) Neurokinin-1 receptor antagonists (NK-1 antagonists) such as the compounds disclosed in: U.S. Pat. Nos. 5,162,339, 5,232,929, 5,242,930, 5,373,003, 5,387,595, 5,459,270, 5,494,926, 5,496,833, and 5,637,699.

In another embodiment, the subject compound may be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; citalopram, duloxetine, fluoxetine, fluvoxamine, paroxetine and sertraline; isocarboxazid, phenelzine, tranylcypromine and selegiline; moclobemide: venlafaxine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

In another embodiment, the subject compound may be employed in combination with anti-Alzheimer's agents; beta-secretase inhibitors, such as verubecestat; gamma-secretase inhibitors; growth hormone secretagogues; recombinant growth hormone; HMG-CoA reductase inhibitors; NSAID's including ibuprofen; vitamin E; anti-amyloid antibodies; CB-1 receptor antagonists or CB-1 receptor inverse agonists; antibiotics such as doxycycline and rifampin; N-methyl-D-aspartate (NMDA) receptor antagonists, such as memantine; cholinesterase inhibitors such as galantamine, rivastigmine, donepezil, and tacrine; growth hormone secretagogues such as ibutamoren, ibutamoren mesylate, and capromorelin; histamine H₃ antagonists; AMPA agonists; PDE IV inhibitors; GABA_{A} inverse agonists; or neuronal nicotinic agonists.

In another embodiment, the subject compound may be employed in combination with sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, clonazepam, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem, and salts thereof, and combinations thereof, and the like, or the subject compound may be administered in conjunction with the use of physical methods such as with light therapy or electrical stimulation.

In another embodiment, the subject compound may be employed in combination with acetophenazine, alentemol, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, risperidone, sulpiride, tetrabenazine, trihexyphenidyl, thioridazine, thiothixene or trifluoperazine.

In another embodiment, the subject compound may be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone.

In another embodiment, the subject compound may be employed in combination with a nicotine agonist or a nicotine receptor partial agonist such as varenicline, opioid antagonists (e.g., naltrexone (including naltrexone depot), antabuse, and nalmefene), dopaminergic agents (e.g., apomorphine), ADD/ADHD agents (e.g., methylphenidate hydrochloride (e.g., Ritalin® and Concerta®), atomoxetine (e.g., Strattera®), a monoamine oxidase inhibitor (MAOI), amphetamines (e.g., Adderall®)) and anti-obesity agents, such as apo-B/MTP inhibitors, 11Beta-hydroxy steroid dehydrogenase-1 (11Beta-HSD type 1) inhibitors, peptide YY3-36 or analogs thereof, MCR-4 agonists, CCK-A agonists, monoamine reuptake inhibitors, sympathomimetic agents, β3 adrenergic receptor agonists, dopamine receptor agonists, melanocyte-stimulating hormone receptor analogs, 5-HT2c receptor agonists, melanin concentrating hormone receptor antagonists, leptin, leptin analogs, leptin receptor agonists, galanin receptor antagonists, lipase inhibitors, bombesin receptor agonists, neuropeptide-Y receptor antagonists (e.g., NPY Y5 receptor antagonists), thyromimetic agents, dehydroepiandrosterone or analogs thereof, glucocorticoid receptor antagonists, orexin receptor antagonists, such as suvorexant, other orexin agonists, glucagon-like peptide-1 receptor agonists, ciliary neurotrophic factors, human agouti-related protein antagonists, ghrelin receptor antagonists, histamine 3 receptor antagonists or inverse agonists, and neuromedin U receptor agonists, and pharmaceutically acceptable salts thereof.

In another embodiment, the subject compound may be employed in combination with an agent such as aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; selective serotonin reuptake inhibitor (SSRI); halogenated amphetamine derivatives, including chlorphentermine, cloforex, clortermine, dexfenfluramine, fenfluramine, picilorex and sibutramine; and pharmaceutically acceptable salts thereof.

In another embodiment, the subject compound may be employed in combination with an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, asprin, codiene, fentanyl, ibuprofen, indomethacin, ketorolac, morphine, naproxen, phenacetin, piroxicam, a steroidal analgesic, sufentanyl, sunlindac, tenidap, and the like. Similarly, the subject compound may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention may be effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives. Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

Several methods for preparing the compounds of this invention are illustrated in the following Schemes and Examples. Starting materials are made according to procedures known in the art or as illustrated herein. The following abbreviations are used herein: Me: methyl; Et: ethyl; t-Bu: tert-butyl; Ar: aryl; Ph: phenyl; Bn: benzyl; Ac: acetyl; ACN: acetonitrile; DAST: Diethylaminosulfur trifluoride; DCM (CH₂Cl₂): dichloromethane; DIPEA: N,N-diisopropylethylamine; DMAP: 4-dimethylaminopyridine; DMF: N,N-dimethylformamide; DMSO: dimethylsulfoxide; DPPF: 1,1′-Bis(diphenylphosphino)ferrocene; EA: Ethyl acetate; EDC: N-(3-Dimethylaminopropyl)-N′-ethylcarbodiimide; Et₃N: triethylamine; EtOAc: ethyl acetate; EtOH: ethanol; Et₃N: Triethylamine; h: hour(s); HABT: 1-Hydroxy-7-azabenzotriazole; HATU: (1-[Bis(dimethylamino)methylene]-1H-1,2,3-triazolo[4,5-b]pyridinium 3-oxide hexafluorophosphate; HCl: hydrogen chloride; HOBT: hydroxybenzotriazole hydrate; HPLC: high performance liquid chromatography; LRMS: Low resolution Mass Spectrometer; MeOH: methanol; MgSO₄: magnesium sulfate; Ms: methanesulfonyl; MsCl: methanesulfonyl chloride; Pd(OAc)₂: Palladium(II) acetate; PE: Petroleum ether; SOCl₂: thionyl chloride: SFC: supercritical fluid chromatography; TEA: triethylamine; THF: tetrahydrofuran; TFA: trifluoracetic acid; TLC: Thin-layer chromatography.

The compounds of the present invention can be prepared in a variety of fashions. In some cases the final product may be further modified, for example, by manipulation of substituents. These manipulations may include, but are not limited to, reduction, oxidation, alkylation, acylation, and hydrolysis reactions which are commonly known to those skilled in the art. In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### EXAMPLES

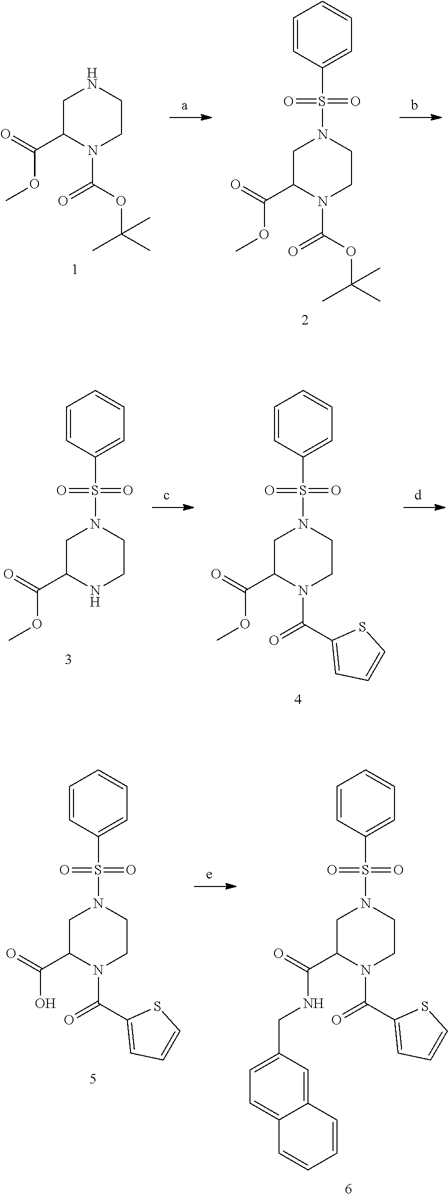 [SMILES: COC(=O)C1CNCCN1C(=O)OC(C)(C)C ; COC(=O)C1CN(S(=O)(=O)c2ccccc2)CCN1C(=O)OC(C)(C)C ; COC(=O)C1CN(S(=O)(=O)c2ccccc2)CCN1 ; COC(=O)C1CN(S(=O)(=O)c2ccccc2)CCN1C(=O)c1cccs1 ; O=C(O)C1CN(S(=O)(=O)c2ccccc2)CCN1C(=O)c1cccs1 ; O=C(NCc1ccc2ccccc2c1)C1CN(S(=O)(=O)c2ccccc2)CCN1C(=O)c1cccs1]

### Preparation of N-(naphthalen-2-ylmethyl)-4-(phenylsulfonyl)-1-(thiophene-2-carbonyl)piperazine-2-carboxamide (6)

Step 1: Preparation of 1-(tert-butyl) 2-methyl 4-(phenylsulfonyl)piperazine-1,2-dicarboxylate (2) To a stirred solution of 1-tert-butyl 2-methyl piperazine-1,2-dicarboxylate (1, 2.50 g, 10.23 mmol) in DCM (34.1 mL) was added pyridine (2.48 mL, 30.70 mmol) and benzene sulfonyl chloride (1.30 mL, 10.23 mmol) at room temperature. The resulting solution was stirred 2 hours, washed with saturated sodium bicarbonate solution, washed with IN HCl solution and the organic phase concentrated. The crude mixture was flash column purified using a 0-30% ethyl acetate/hexane gradient to give 1-(tert-butyl) 2-methyl 4-(phenylsulfonyl)piperazine-1,2-dicarboxylate (2) LRMS (ES) (M+H)⁺: observed=329.2(-tbutyl). calculated=384.4.

Step 2: Preparation of methyl 4-(phenylsulfonyl)piperazine-2-carboxylate (3) A stirred solution of 1-(tert-butyl) 2-methyl 4-(phenylsulfonyl)piperazine-1,2-dicarboxylate (2, 0.74 g, 1.93 mmol) in Ethyl Acetate (15 mL) was chilled in an ice bath to 0° C. To this solution was bubbled in HCl gas. The solution was stirred 30 min and concentrated to give methyl 4-(phenylsulfonyl)piperazine-2-carboxylate (3) as an HCl salt. LRMS (ES) (M+H)⁺: observed=285.2. calculated=284.3.

Step 3: Preparation of methyl 4-(phenylsulfonyl)-1-(thiophene-2-carbonyl)piperazine-2-carboxylate (4). To a stirred solution of methyl 4-(phenylsulfonyl)piperazine-2-carboxylate (3, 0.62 g, 1.92 mmol) and thiophene-2-carboxylic acid (0.25 g, 1.92 mmol) in DCM (5.5 mL) was added DMAP (0.47 g, 3.85 mmol) and EDC (0.44 g, 2.31 mmol). The resulting mixture was stirred 2 hours, then washed with 1N HCl solution and concentrated. Flash column purification using a 0-80% ethyl acetate/hexane gradient gave methyl 4-(phenylsulfonyl)-1-(thiophene-2-carbonyl)piperazine-2-carboxylate (4). LRMS (ES) (M+H)⁺: observed=395.2. calculated=394.5.

Step 4: Preparation of 4-(phenylsulfonyl)-1-(thiophene-2-carbonyl)piperazine-2-carboxylic acid (5). To a stirred solution of methyl 4-(phenylsulfonyl)-1-(thiophene-2-carbonyl)piperazine-2-carboxylate (4, 0.48 g 1.23 mmol) in a mixture of THF (6.1 mL) and methanol (6.1 mL) was added lithium hydroxide solution (1M, 3.46 mL, 3.46 mmol). The resulting mixture was heated to 55° C. for 2 hours. The reaction was concentrated and partitioned between EtOAc and 1N HCl solution. The organic phase was concentrated to give 4-(phenylsulfonyl)-1-(thiophene-2-carbonyl)piperazine-2-carboxylic acid (5). LRMS (ES) (M+H)⁺: observed=381.2. calculated=380.4.

Step 5: Preparation of N-(naphthalen-2-ylmethyl)-4-(phenylsulfonyl)-1-(thiophene-2-carbonyl)piperazine-2-carboxamide (6) To a stirred solution of 4-(phenylsulfonyl)-1-(thiophene-2-carbonyl)piperazine-2-carboxylic acid (5, 0.025 g, 0.066 mmol) and naphthalen-2-ylmethanamine HCl (0.015 g, 0.07 mmol) in DMF (0.5 mL) was added HATU (0.025 g, 0.066 mmol) followed by triethylamine (0.027 mL, 0.197 mmol). The resulting solution was stirred 20 minutes and purified by reverse phase purification to give N-(naphthalen-2-ylmethyl)-4-(phenylsulfonyl)-1-(thiophene-2-carbonyl)piperazine-2-carboxamide (6). LRMS (ES) (M+H)⁺: observed=520.1. calculated=519.6.

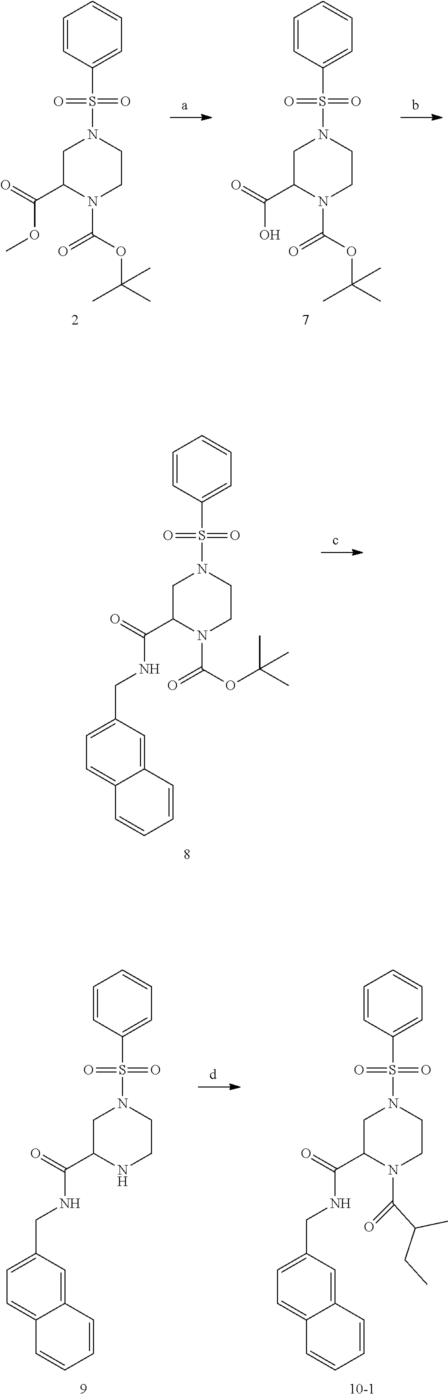 [SMILES: COC(=O)C1CN(S(=O)(=O)c2ccccc2)CCN1C(=O)OC(C)(C)C ; CC(C)(C)OC(=O)N1CCN(S(=O)(=O)c2ccccc2)CC1C(=O)O ; CC(C)(C)OC(=O)N1CCN(S(=O)(=O)c2ccccc2)CC1C(=O)NCc1ccc2ccccc2c1 ; O=C(NCc1ccc2ccccc2c1)C1CN(S(=O)(=O)c2ccccc2)CCN1 ; CCC(C)C(=O)N1CCN(S(=O)(=O)c2ccccc2)CC1C(=O)NCc1ccc2ccccc2c1]

### Preparation of 1-(2-methylbutanoyl)-N-(naphthalen-2-ylmethyl)-4-(phenylsulfonyl)piperazine-2-carboxamide (10-1)

Step 1: Preparation of 1-(tert-butoxycarbonyl)-4-(phenylsulfonyl)piperazine-2-carboxylic acid (7) In To a stirred solution of 1-(tert-butyl) 2-methyl 4-(phenylsulfonyl)piperazine-1,2-dicarboxylate (2, 2.92 g 7.60 mmol) in a mixture of THF (31.6 mL) and methanol (31.6 mL) was added lithium hydroxide solution (1M, 15.19 mL, 15.19 mmol). The resulting mixture was heated to 55° C. for 18 hours. The reaction was concentrated and partitioned between EtOAc and 1N HCl solution. The organic phase was concentrated and dried over sodium sulfate to 1-(tert-butoxycarbonyl)-4-(phenylsulfonyl)piperazine-2-carboxylic acid (7). LRMS (ES) (M+H)⁺: observed=315.2(-tbutyl). calculated=370.4.

Step 2: Preparation of tert-butyl 2-((naphthalen-2-ylmethyl)carbamoyl)-4-(phenylsulfonyl)piperazine-1-carboxylate (8) To a stirred solution of 1-(tert-butoxycarbonyl)-4-(phenylsulfonyl)piperazine-2-carboxylic acid (7, 0.05 g, 1.35 mmol) and naphthalen-2-ylmethanamine (0.23 g, 1.47 mmol) in DCM (3.8 mL) was added DMAP (0.33 g, 2.70 mmol) and EDC (0.31 g, 1.62 mmol). The resulting mixture was stirred 1 hours, then washed with 1N HCl solution and concentrated. Flash column purification using a 0-50% ethyl acetate/hexane gradient gave tert-butyl 2-((naphthalen-2-ylmethyl)carbamoyl)-4-(phenylsulfonyl)piperazine-1-carboxylate (8). LRMS (ES) (M+H)⁺: observed=510.4. calculated=509.6.

Step 3: Preparation of N-(naphthalen-2-ylmethyl)-4-(phenylsulfonyl)piperazine-2-carboxamide 2) A stirred solution of tert-butyl 2-((naphthalen-2-ylmethyl)carbamoyl)-4-(phenylsulfonyl)piperazine-1-carboxylate (8, 1.35 mmol theoretical) in Ethyl Acetate (7 mL) was chilled in an ice bath to 0° C. To this solution was bubbled in HCl gas. The solution was stirred 30 min and concentrated to give N-(naphthalen-2-ylmethyl)-4-(phenylsulfonyl)piperazine-2-carboxamide (9) as an HCl salt. LRMS (ES) (M+H)⁺: observed=410.3. calculated=409.5.

Step 4: Preparation of 1-(2-methylbutanoyl)-N-(naphthalen-2-ylmethyl)-4-(phenylsulfonyl)piperazine-2-carboxamide (10-1) To a stirred solution of N-(naphthalen-2-ylmethyl)-4-(phenylsulfonyl)piperazine-2-carboxamide (9, 0.03 g, 0.067 mmol) and 2-methylbutanoic acid (0.0075 g, 0.074 mmol) in DMF (0.34 mL) was added HATU (0.028 g, 0.074 mmol) followed by triethylamine (0.028 mL, 0.20 mmol). The resulting solution was stirred 20 minutes and purified by reverse phase purification to give 1-(2-methylbutanoyl)-N-(naphthalen-2-ylmethyl)-4-(phenylsulfonyl)piperazine-2-carboxamide (10-1). LRMS (ES) (M+H)⁺: observed=494.3. calculated=493.6.

The following compounds were prepared in an analagous manner to example 10-1.

| | | | Observed | |
|---|---|---|---|---|
| | | | Mass | Calculated |
| Example | Compound | Structure | (M + H) | Mass |
| 10-2 | N-(naphthalen-2- ylmethyl)-4- (phenylsulfonyl)-1- propionylpiperazine- 2-carboxamide | 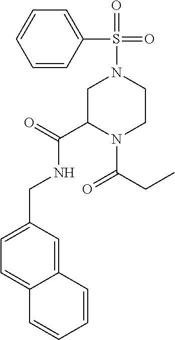 [SMILES: CCC(=O)N1CCN(S(=O)(=O)c2ccccc2)CC1C(=O)NCc1ccc2ccccc2c1] | 466.1 | 465.576 |
| 10-3 | 1-butyryl-N- (naphthalen-2- ylmethyl)-4- (phenylsulfonyl) piperazine-2- carboxamide | 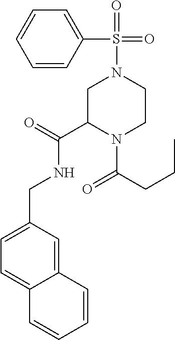 [SMILES: CCCC(=O)N1CCN(S(=O)(=O)c2ccccc2)CC1C(=O)NCc1ccc2ccccc2c1] | 480.1 | 479.603 |
| 10-4 | 1-isobutyryl-N- (naphthalen-2- ylmethyl)-4- (phenylsulfonyl) piperazine-2- carboxamide | 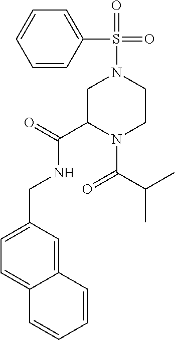 [SMILES: CC(C)C(=O)N1CCN(S(=O)(=O)c2ccccc2)CC1C(=O)NCc1ccc2ccccc2c1] | 480.1 | 479.603 |
| 10-5 | 1- (cyclopropane- carbonyl)-N-(naphthalen- 2-ylmethyl)-4- (phenylsulfonyl) piperazine-2- carboxamide | 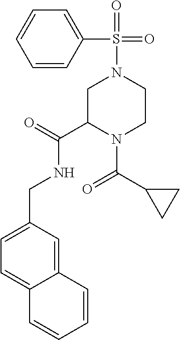 [SMILES: O=C(NCc1ccc2ccccc2c1)C1CN(S(=O)(=O)c2ccccc2)CCN1C(=O)C1CC1] | 478.1 | 477.587 |
| 10-6 | 1- (cyclobutanecarbonyl)- N-(naphthalen- 2-ylmethyl)-4- (phenylsulfonyl) piperazine-2- carboxamide | 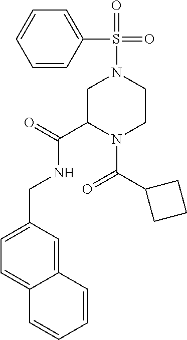 [SMILES: O=C(NCc1ccc2ccccc2c1)C1CN(S(=O)(=O)c2ccccc2)CCN1C(=O)C1CCC1] | 492.1 | 491.614 |
| 10-7 | N-(naphthalen-2- ylmethyl)-4- (phenylsulfonyl)-1- (thiazole-2- carbonyl)piperazine- 2-carboxamide | 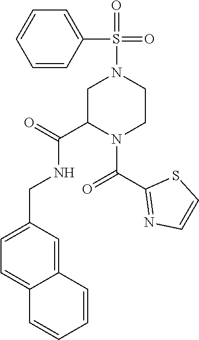 [SMILES: O=C(NCc1ccc2ccccc2c1)C1CN(S(=O)(=O)c2ccccc2)CCN1C(=O)c1nccs1] | 521.1 | 520.634 |
| 10-8 | N-(naphthalen-2- ylmethyl)-4- (phenylsulfonyl)-1- (thiophene-3- carbonyl)piperazine-2- carboxamide | 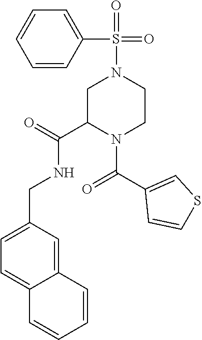 [SMILES: O=C(NCc1ccc2ccccc2c1)C1CN(S(=O)(=O)c2ccccc2)CCN1C(=O)c1ccsc1] | 520.4 | 519.646 |
| 10-9 | 1-(furan-2- carbonyl)-N- (naphthalen-2- ylmethyl)-4- (phenylsulfonyl) piperazine-2- carboxamide | 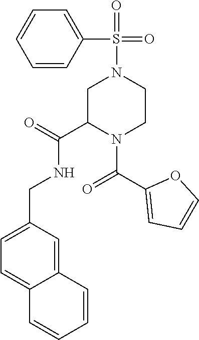 [SMILES: O=C(NCc1ccc2ccccc2c1)C1CN(S(=O)(=O)c2ccccc2)CCN1C(=O)c1ccco1] | 504.1 | 503.581 |
| 10-10 | N-(naphthalen-2- ylmethyl)-4- (phenylsulfonyl)-1- (thiazole-5- carbonyl)piperazine-2- carboxamide | 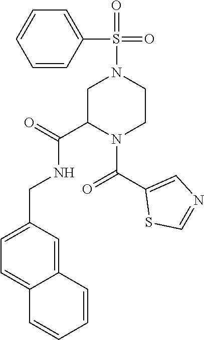 [SMILES: O=C(NCc1ccc2ccccc2c1)C1CN(S(=O)(=O)c2ccccc2)CCN1C(=O)c1cncs1] | 521.1 | 520.634 |
| 10-11 | 1-(2- methylbenzoyl)-N- (naphthalen-2- ylmethyl)-4- (phenylsulfonyl) piperazine-2- carboxamide | 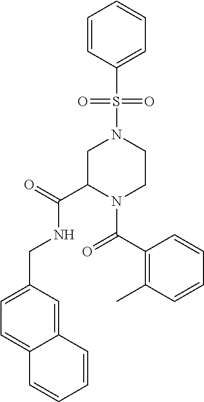 [SMILES: Cc1ccccc1C(=O)N1CCN(S(=O)(=O)c2ccccc2)CC1C(=O)NCc1ccc2ccccc2c1] | 528.2 | 527.647 |
| 10-12 | 1-(3- methylthiophene-2- carbonyl)-N- (naphthalen-2- ylmethyl)-4- (phenylsulfonyl) piperazine-2- carboxamide | 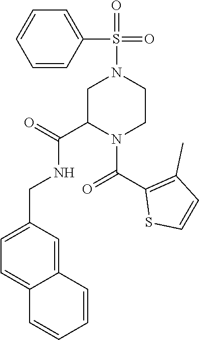 [SMILES: Cc1ccsc1C(=O)N1CCN(S(=O)(=O)c2ccccc2)CC1C(=O)NCc1ccc2ccccc2c1] | 534.1 | 533.673 |

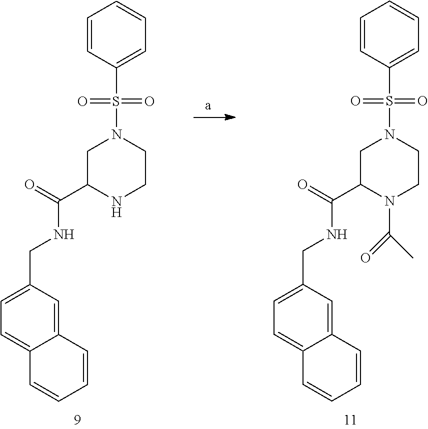 [SMILES: O=C(NCc1ccc2ccccc2c1)C1CN(S(=O)(=O)c2ccccc2)CCN1 ; CC(=O)N1CCN(S(=O)(=O)c2ccccc2)CC1C(=O)NCc1ccc2ccccc2c1]

### Preparation of 1-acetyl-N-(naphthalen-2-ylmethyl)-4-(phenylsulfonyl)piperazine-2-carboxamide (11)

To a stirred solution of N-(naphthalen-2-ylmethyl)-4-(phenylsulfonyl)piperazine-2-carboxamide (9, 0.03 g, 0.067 mmol) in DCM (0.3 mL) was added acetic anhydride (0.007 mL, 0.074 mmol) and triethylamine (0.028 mL, 0.20 mmol) and the resulting solution was stirred at room temperature for 1 hour and concentrated. Purification with reverse phase chromotography provided 1-acetyl-N-(naphthalen-2-ylmethyl)-4-(phenylsulfonyl)piperazine-2-carboxamide (11). LRMS (ES) (M+H)⁺: observed=452.3. calculated=451.5.

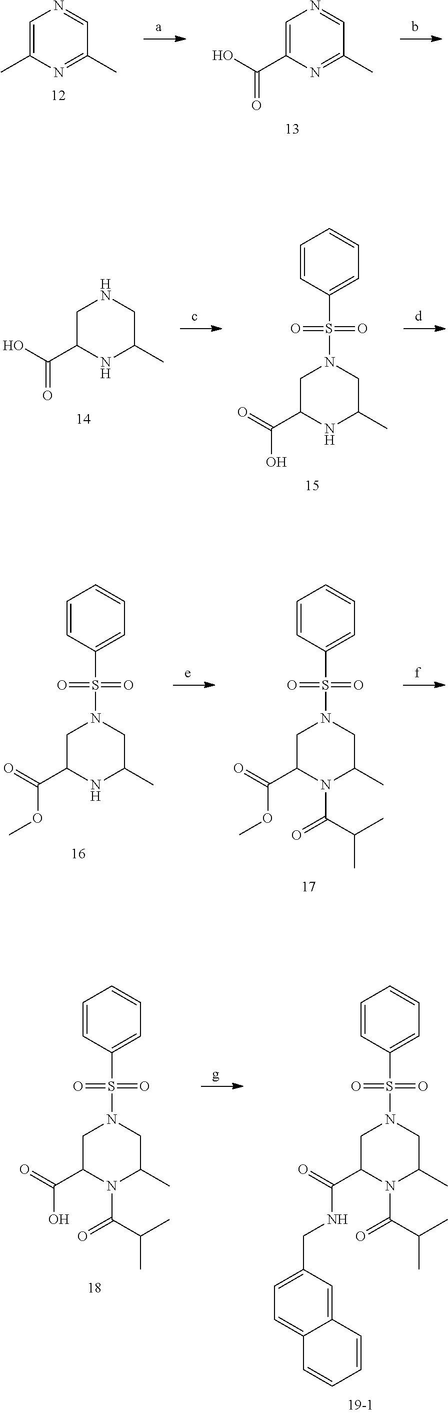

### Preparation of cis-1-isobutyryl-6-methyl-N-(naphthalen-2-ylmethyl)-4-(phenylsulfonyl)piperazine-2-carboxamide (19-1)

Step 1: Preparation of 6-methylpyrazine-2-carboxylic acid (13) Into a 20-L 4-necked round-bottom flask purged and maintained with an inert atmosphere of nitrogen, was placed a solution of 2,6-dimethylpyrazine (12,400 g, 3.70 mol) in water (4 L). This was followed by the addition of a solution of KMnO4 (1287 g, 8.15 mol) in water (10 L), dropwise at 70° C. The resulting solution was stirred overnight at 70-75° C. The solids were filtered out. The pH value of the solution was adjusted to 1.5 with 5N hydrogen chloride solution. The resulting solution was extracted with 10×5 L of ethyl acetate and the organic layers combined and dried over anhydrous sodium sulfate and concentrated under vacuum. This gave 6-methylpyrazine-2-carboxylic acid (13).

Step 2: Preparation of cis-6-methylpiperazine-2-carboxylic acid (14) Into a 5-L pressure tank reactor purged, was placed a solution of sodium hydroxide (92.7 g, 2.32 mol, 2.00 equiv) in water (1.6 L), 6-methylpyrazine-2-carboxylic acid (13, 160 g, 1.16 mol, 1.00 equiv), Palladium carbon (64 g). To the above 150 psi H2 (g) was introduced. The resulting solution was stirred for 48 h at 50° C. The solids were filtered out.

Step 3: Preparation of cis-6-methyl-4-(phenylsulfonyl)piperazine-2-carboxylic acid (15) Into a 5-L 4-necked round-bottom flask purged and maintained with an inert atmosphere of nitrogen, was placed a solution of cis-6-methylpiperazine-2-carboxylic acid (14, 150 g, 1.04 mol, 1.00 equiv) in tetrahydrofuran/H₂O (0.8/1.6 L). This was followed by the addition of benzenesulfonyl chloride (183 g, 1.04 mol) dropwise with stirring at 0° C. The resulting solution was stirred for 2 h at room temperature. The resulting mixture was concentrated under vacuum to give cis-6-methyl-4-(phenylsulfonyl)piperazine-2-carboxylic acid (15).

Step 4: Preparation of cis-methyl 6-methyl-4-(phenylsulfonyl)piperazine-2-carboxylate (16) Into a 10-L 4-necked round-bottom flask purged and maintained with an inert atmosphere of nitrogen, was placed a solution of cis-6-methyl-4-(phenylsulfonyl)piperazine-2-carboxylic acid (15, 260 g, 914.43 mmol) in methanol (4 L). This was followed by the addition of sulfuric acid (538 g, 5.49 mol) dropwise with stirring. The resulting solution was stirred overnight at 80° C. The reaction mixture was cooled with a water/ice bath. The reaction was then quenched by the addition of 4 L of water/ice. The pH value of the solution was adjusted to 8 with sodium bicarbonate solution. The resulting solution was extracted with 3×3 L of dichloromethane and the organic layers combined. The resulting mixture was washed with 1×4 L of brine. The mixture was dried over anhydrous sodium sulfate and concentrated under vacuum. The residue was applied onto a silica gel column with ethyl acetate/petroleum ether (1:3). The crude product (80 g) was purified by Flash-Prep-HPLC with the following conditions (IntelFlash-1): Column, silica gel; mobile phase, ACN/H₂O=10% ACN increasing to ACN/H₂O=40% ACN within 30 min; Detector, UV 210 nm. This gave cis-methyl 6-methyl-4-(phenylsulfonyl)piperazine-2-carboxylate (16). LRMS (ES) (M+H)⁺: observed=299.0. calculated=298.3.

Step 5: Preparation of cis-methyl 1-isobutyryl-6-methyl-4-(phenylsulfonyl)piperazine-2-carboxylate (17) To a stirred solution of cis-methyl 6-methyl-4-(phenylsulfonyl)piperazine-2-carboxylate (16, 4.0 g, 13.41 mmol) in DCM (38.3 mL) was added triethylamine (3.74 mL, 26.8 mmol) and isobutyryl chloride (1.54 mL, 14.75 mmol). The resulting solution was stirred at room temperature for 1 hr, washed with 1N HCl solution, dried over sodium sulfate and concentrated to give cis-methyl 1-isobutyryl-6-methyl-4-(phenylsulfonyl)piperazine-2-carboxylate (17)

Step 6: Preparation of cis-1-isobutyryl-6-methyl-4-(phenylsulfonyl)piperazine-2-carboxylic acid (18) A stirred solution of cis-methyl 1-isobutyryl-6-methyl-4-(phenylsulfonyl)piperazine-2-carboxylate (17, 3.61 g, 9.8 mmol) in THF (49 mL) was chilled in an ice bath to 0° C. To this was added potassium trimethylsilanolate (3.07 g, 21.56 mmol) and the resulting solution was stirred at 0° C. for three hours. The reaction was quenched using 1N HCl solution and extracted several times with ethyl acetate. The organic layers were combined and washed with brine and concentrated. Flash column purification using a 0-20% isopropanol/DCM gradient gave cis-1-isobutyryl-6-methyl-4-(phenylsulfonyl)piperazine-2-carboxylic acid (18)

Step 7: Preparation of cis-1-isobutyryl-6-methyl-N-(naphthalen-2-ylmethyl)-4-(phenylsulfonyl)piperazine-2-carboxamide (19-1) To a stirred solution of cis-1-isobutyryl-6-methyl-4-(phenylsulfonyl)piperazine-2-carboxylic acid (18, 0.033 g, 0.093 mmol) and naphthalen-2-ylmethanamine (0.018 g, 0.101 mmol) in DMF (0.5 mL) was added HATU (0.043 g, 0.112 mmol) followed by triethylamine (0.039 mL, 0.28 mmol). The resulting solution was stirred 20 minutes and purified by reverse phase purification to give cis-1-isobutyryl-6-methyl-N-(naphthalen-2-ylmethyl)-4-(phenylsulfonyl)piperazine-2-carboxamide (19-1). LRMS (ES) (M+H)⁺: observed=494.0. calculated=493.6.

The following compounds were prepared in an analagous manner to example 19-1.

| | | | Observed | |
|---|---|---|---|---|
| | | | Mass | Calculated |
| Example | Compound | Structure | (M + H) | Mass |
| 19-2 | cis-N-(4-(furan-2- yl)benzyl)-1-isobutyryl-6- methyl-4- (phenylsulfonyl)piperazine-2- carboxamide | 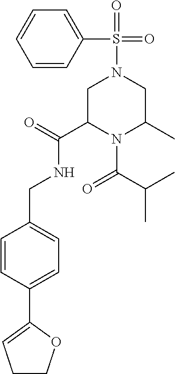 [SMILES: CC(C)C(=O)N1C(C)CN(S(=O)(=O)c2ccccc2)CC1C(=O)NCc1ccc(C2=CCCO2)cc1] | 510.3 | 509.629 |
| 19-3 | cis-N-([1,1′-biphenyl]-4- ylmethyl)-1-isobutyryl-6- methyl-4- (phenylsulfonyl)piperazine-2- carboxamide | 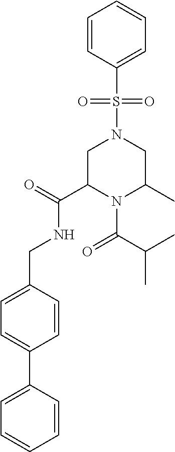 [SMILES: CC(C)C(=O)N1C(C)CN(S(=O)(=O)c2ccccc2)CC1C(=O)NCc1ccc(-c2ccccc2)cc1] | 520.4 | 519.668 |
| 19-4 | cis-N-(4-(1,1- difluoroethoxy)benzyl)-1- isobutyryl-6-methyl-4- (phenylsulfonyl)piperazine- 2-carboxamide | 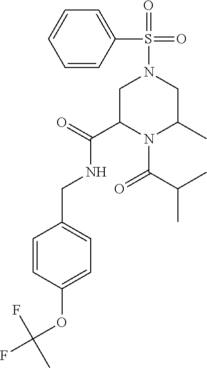 [SMILES: CC(C)C(=O)N1C(C)CN(S(=O)(=O)c2ccccc2)CC1C(=O)NCc1ccc(OC(C)(F)F)cc1] | 524.3 | 523.604 |
| 19-5 | cis-1-isobutyryl-6-methyl- 4-(phenylsulfonyl)-N-(4- (pyrimidin-2- yl)benzyl)piperazine-2- carboxamide | 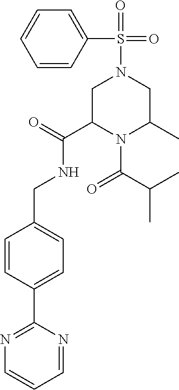 [SMILES: CC(C)C(=O)N1C(C)CN(S(=O)(=O)c2ccccc2)CC1C(=O)NCc1ccc(-c2ncccn2)cc1] | 522.4 | 521.643 |
| 19-6 | cis-1-isobutyryl-6-methyl- 4-(phenylsulfonyl)-N-(4- (thiazol-2- yl)benzyl)piperazine-2- carboxamide | 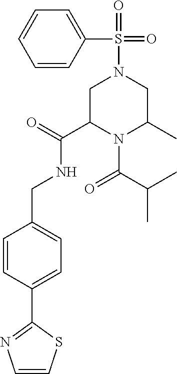 [SMILES: CC(C)C(=O)N1C(C)CN(S(=O)(=O)c2ccccc2)CC1C(=O)NCc1ccc(-c2nccs2)cc1] | 527 | 526.681 |
| 19-7 | cis-N-(4-(2H-1,2,3-triazol- 2-yl)benzyl)-1-isobutyryl- 6-methyl-4- (phenylsulfonyl)piperazine-2- carboxamide | 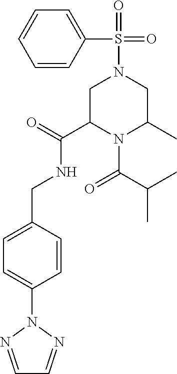 [SMILES: CC(C)C(=O)N1C(C)CN(S(=O)(=O)c2ccccc2)CC1C(=O)NCc1ccc(-n2nccn2)cc1] | 511.4 | 510.62 |
| 19-8 | cis-1-isobutyryl-6-methyl- N-(4-(oxazol-2-yl)benzyl)- 4- (phenylsulfonyl)piperazine-2- carboxamide | 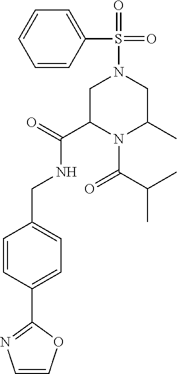 [SMILES: CC(C)C(=O)N1C(C)CN(S(=O)(=O)c2ccccc2)CC1C(=O)NCc1ccc(-c2ncco2)cc1] | 511 | 510.617 |
| 19-9 | cis-N-(4-(1H-pyrazol-1- yl)benzyl)-1-isobutyryl-6- methyl-4- (phenylsulfonyl)piperazine- 2-carboxamide | 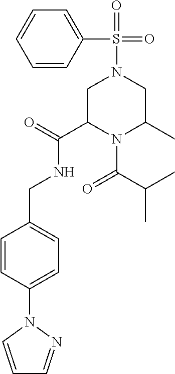 [SMILES: CC(C)C(=O)N1C(C)CN(S(=O)(=O)c2ccccc2)CC1C(=O)NCc1ccc(-n2cccn2)cc1] | 510 | 509.632 |
| 19-10 | cis-1-isobutyryl-6-methyl- 4-(phenylsulfonyl)-N-(4- propylbenzyl)piperazine-2- carboxamide | 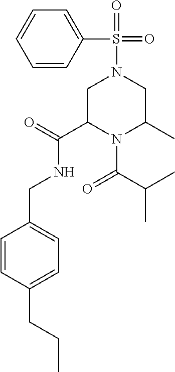 [SMILES: CCCc1ccc(CNC(=O)C2CN(S(=O)(=O)c3ccccc3)CC(C)N2C(=O)C(C)C)cc1] | 486.2 | 485.651 |
| 19-11 | cis-N-(4-ethoxybenzyl)-1- isobutyryl-6-methyl-4- (phenylsulfonyl)piperazine- 2-carboxamide | 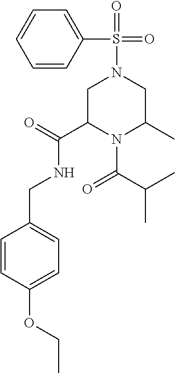 [SMILES: CCOc1ccc(CNC(=O)C2CN(S(=O)(=O)c3ccccc3)CC(C)N2C(=O)C(C)C)cc1] | 488.2 | 487.623 |

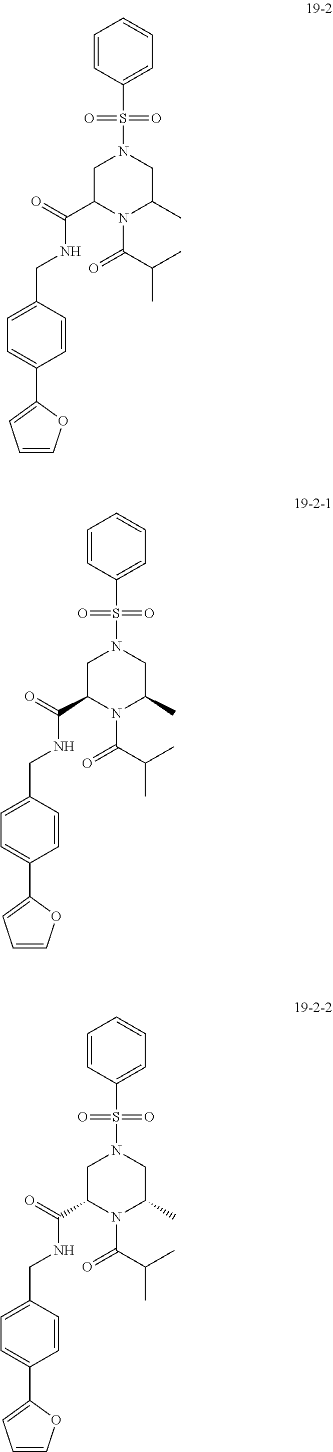 [SMILES: CC(C)C(=O)N1C(C)CN(S(=O)(=O)c2ccccc2)CC1C(=O)NCc1ccc(-c2ccco2)cc1 ; CC(C)C(=O)N1[C@H](C)CN(S(=O)(=O)c2ccccc2)C[C@@H]1C(=O)NCc1ccc(-c2ccco2)cc1 ; CC(C)C(=O)N1[C@@H](C)CN(S(=O)(=O)c2ccccc2)C[C@H]1C(=O)NCc1ccc(-c2ccco2)cc1]

### Separation of cis-N-(4-(furan-2-yl)benzyl)-1-isobutyryl-4-(phenylsulfonyl)-6-(trifluoromethyl)piperazine-2-carboxamide (19-2)

cis-N-(4-(furan-2-yl)benzyl)-1-isobutyryl-4-(phenylsulfonyl)-6-(trifluoromethyl)piperazine-2-carboxamide (19-2, 0.262 g, 0.514 mmol) was separated using SFC by injecting (0.8 mL) 2 mg/mL methanol to an AD-H (2×25 cm) column using a 35% ethanol/CO₂ gradient at 100 bar, flow rate of 60 mL/min to give (2R,6R)—N-(4-(furan-2-yl)benzyl)-1-isobutyryl-6-methyl-4-(phenylsulfonyl)piperazine-2-carboxamide (19-2-1, PEAK 1) and (2S,6S)—N-(4-(furan-2-yl)benzyl)-1-isobutyryl-6-methyl-4-(phenylsulfonyl)piperazine-2-carboxamide (19-2-2, PEAK 2).

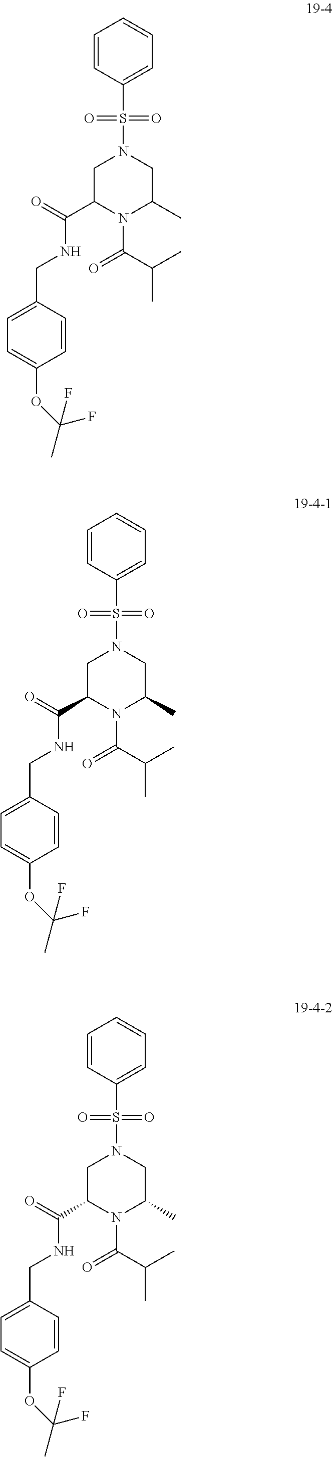 [SMILES: CC(C)C(=O)N1C(C)CN(S(=O)(=O)c2ccccc2)CC1C(=O)NCc1ccc(OC(C)(F)F)cc1 ; CC(C)C(=O)N1[C@H](C)CN(S(=O)(=O)c2ccccc2)C[C@@H]1C(=O)NCc1ccc(OC(C)(F)F)cc1 ; CC(C)C(=O)N1[C@@H](C)CN(S(=O)(=O)c2ccccc2)C[C@H]1C(=O)NCc1ccc(OC(C)(F)F)cc1]

### Separation of cis-N-(4-(1,1-difluoroethoxy)benzyl)-1-isobutyryl-6-methyl-4-(phenylsulfonyl)piperazine-2-carboxamide (19-4)

In an analogous manner to 19-2, cis-N-(4-(1,1-difluoroethoxy)benzyl)-1-isobutyryl-6-methyl-4-(phenylsulfonyl)piperazine-2-carboxamide (19-4) was separated by SFC using a 20% ethanol/CO₂ gradient gave (2R,6R)—N-(4-(1,1-difluoroethoxy)benzyl)-1-isobutyryl-6-methyl-4-(phenylsulfonyl)piperazine-2-carboxamide (19-4-1, PEAK 1) and (2S,6S)—N-(4-(1,1-difluoroethoxy)benzyl)-1-isobutyryl-6-methyl-4-(phenylsulfonyl)piperazine-2-carboxamide (19-4-2, PEAK 2).

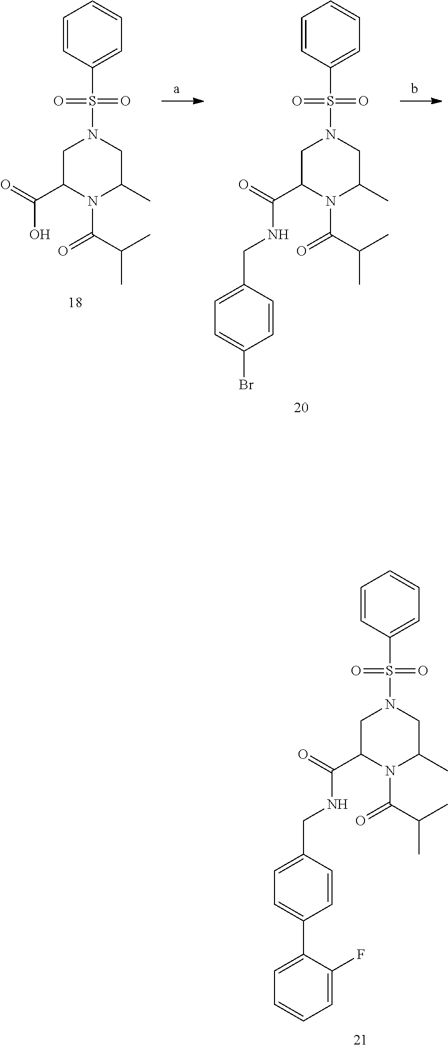 [SMILES: CC(C)C(=O)N1C(C)CN(S(=O)(=O)c2ccccc2)CC1C(=O)O ; CC(C)C(=O)N1C(C)CN(S(=O)(=O)c2ccccc2)CC1C(=O)NCc1ccc(Br)cc1 ; CC(C)C(=O)N1C(C)CN(S(=O)(=O)c2ccccc2)CC1C(=O)NCc1ccc(-c2ccccc2F)cc1]

### Preparation of cis-N-((2′-fluoro-[1,1′-biphenyl]-4-yl)methyl)-1-isobutyryl-6-methyl-4-(phenylsulfonyl)piperazine-2-carboxamide (21)

Step 1: Preparation of cis-N-(4-bromobenzyl)-1-isobutyryl-6-methyl-4-(phenylsulfonyl)piperazine-2-carboxamide (20) To a stirred solution of cis-1-isobutyryl-6-methyl-4-(phenylsulfonyl)piperazine-2-carboxylic acid (18, 0.200 g, 0.564 mmol) and (4-bromophenyl)methanamine (0.13 g, 0.68 mmol) in DCM (1.6 mL) was added HABT (0.084 g, 0.62 mmol) and EDC (0.13 g, 0.68 mmol). The resulting mixture was stirred 1 hour 30 minutes, then washed with 1N HCl solution and concentrated. Flash column purification using a 0-60% ethyl acetate/hexane gradient gave cis-N-(4-bromobenzyl)-1-isobutyryl-6-methyl-4-(phenylsulfonyl)piperazine-2-carboxamide (20). LRMS (ES) (M+H)⁺: observed=523.3. calculated=522.4.

Step 2: Preparation of cis-N-(2′-fluoro-[1,1′-biphenyl]-4-yl)methyl)-1-isobutyryl-6-methyl-4-(phenylsulfonyl)piperazine-2-carboxamide (21) To a round bottom flask containing cis-N-(4-bromobenzyl)-1-isobutyryl-6-methyl-4-(phenylsulfonyl)piperazine-2-carboxamide (20, 0.035 g, 0.67 mmol) and 2-(2-fluorophenyl)-4,4,5,5-tetramethyl-1,3,2-dioxaborolane (0.018 g, 0.08 mmol) and Palladium(II) acetate (0.002 g, 6.7 μM) and 2-Dicyclohexylphosphino-2′,6′-dimethoxybiphenyl (0.006 g, 0.013 mmol) and sodium bicarbonate (0.014 g, 0.17 mmol) under nitrogen was added 1,4-dioxane (0.28 mL) and water (0.06 mL). The resulting mixture was stirred and heated to 90° C. for 1 hour and 30 minutes. The reaction was allowed to cool and concentrated. The crude mixture was dissolved in DMSO and reverse phase purification gave cis-N-((2′-fluoro-[1,1′-biphenyl]-4-yl)methyl)-1-isobutyryl-6-methyl-4-(phenylsulfonyl)piperazine-2-carboxamide (21). LRMS (ES) (M+H)⁺: observed=538.4. calculated=537.6.

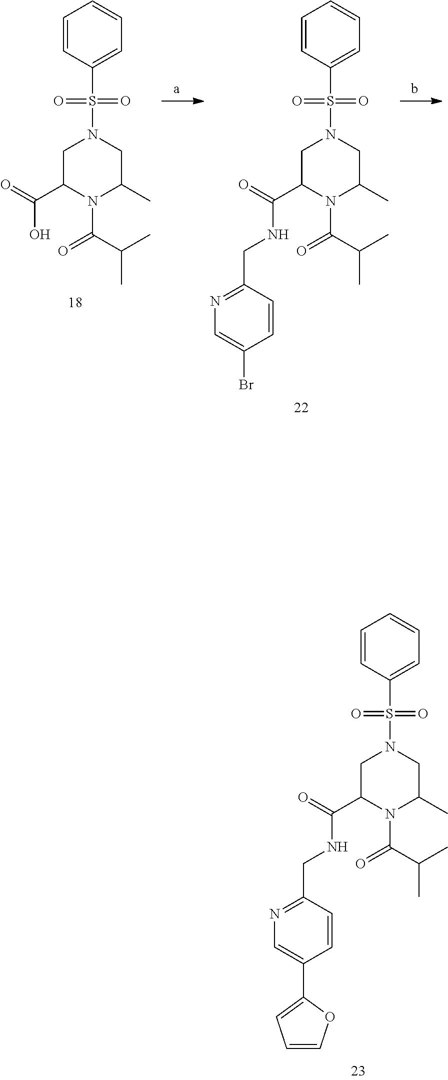 [SMILES: CC(C)C(=O)N1C(C)CN(S(=O)(=O)c2ccccc2)CC1C(=O)O ; CC(C)C(=O)N1C(C)CN(S(=O)(=O)c2ccccc2)CC1C(=O)NCc1ccc(Br)cn1 ; CC(C)C(=O)N1C(C)CN(S(=O)(=O)c2ccccc2)CC1C(=O)NCc1ccc(-c2ccco2)cn1]

### Preparation of cis-N-((5-(furan-2-yl)pyridin-2-yl)methyl)-1-isobutyryl-6-methyl-4-(phenylsulfonyl)piperazine-2-carboxamide (23)

Step 1: Preparation of cis-N-((5-bromopyridin-2-yl)methyl)-1-isobutyryl-6-methyl-4-(phenylsulfonyl)piperazine-2-carboxamide (22) To a stirred solution of cis-1-isobutyryl-6-methyl-4-(phenylsulfonyl)piperazine-2-carboxylic acid (18, 1.50 g, 4.23 mmol) and (5-bromopyridin-2-yl)methanamine, HCl (1.04 g, 4.66 mmol) in DMF (12.0 mL) was added HATU (1.77 g, 4.66 mmol) followed by triethylamine (1.77 mL, 12.70 mmol). The resulting solution was stirred 3 hours, diluted with sodium bicarbonate solution and extracted with ethyl acetate. The organic phase was concentrated and flash column purification using a 0-100% ethyl acetate/hexane gradient gave cis-N-((5-bromopyridin-2-yl)methyl)-1-isobutyryl-6-methyl-4-(phenylsulfonyl)piperazine-2-carboxamide (22). LRMS (ES) (M+H)⁺: observed=525.3. calculated=523.4.

Step 2: Preparation of cis-N-((5-(furan-2-yl)pyridin-2-yl)methyl)-1-isobutyryl-6-methyl-4-(phenylsulfonyl)piperazine-2-carboxamide (23) In an analogous manner to 21, cis-N-((5-bromopyridin-2-yl)methyl)-1-isobutyryl-6-methyl-4-(phenylsulfonyl)piperazine-2-carboxamide and 2-(furan-2-yl)-4,4,5,5-tetramethyl-1,3,2-dioxaborolane were used to prepare cis-N-((5-(furan-2-yl)pyridin-2-yl)methyl)-1-isobutyryl-6-methyl-4-(phenylsulfonyl)piperazine-2-carboxamide (23). LRMS (ES) (M+H)⁺: observed=511.4. calculated=510.6.

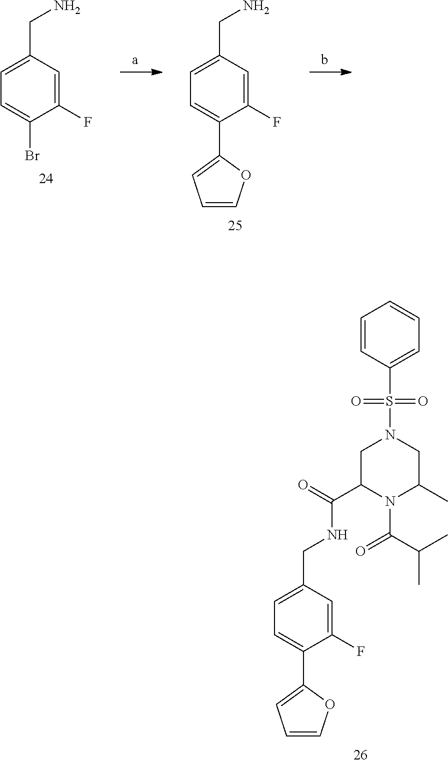 [SMILES: NCc1ccc(Br)c(F)c1 ; NCc1ccc(-c2ccco2)c(F)c1 ; CC(C)C(=O)N1C(C)CN(S(=O)(=O)c2ccccc2)CC1C(=O)NCc1ccc(-c2ccco2)c(F)c1]

### Preparation of cis-N-(3-fluoro-4-(furan-2-yl)benzyl)-1-isobutyryl-6-methyl-4-(phenylsulfonyl)piperazine-2-carboxamide (26)

Step 1: In an analogous manner to 21, (4-bromo-3-fluorophenyl)methanamine (24) and 2-(furan-2-yl)-4,4,5,5-tetramethyl-1,3,2-dioxaborolane were used to prepare (3-fluoro-4-(furan-2-yl)phenyl)methanamine (25, 42%). LRMS (ES) (M+H)⁺: observed=192.1. calculated=191.2.

Step 2: In an analogous manner to 19-1, cis-1-isobutyryl-6-methyl-4-(phenylsulfonyl)piperazine-2-carboxylic acid (18) and (3-fluoro-4-(furan-2-yl)phenyl)methanamine (25) were used to prepare cis-N-(3-fluoro-4-(furan-2-yl)benzyl)-1-isobutyryl-6-methyl-4-(phenylsulfonyl)piperazine-2-carboxamide (26) LRMS (ES) (M+H)⁺: observed=528.3. calculated=527.6.

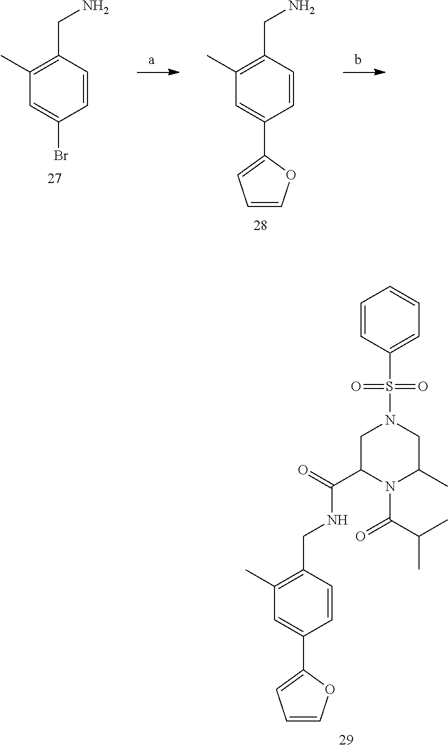

### Preparation of cis-N-(4-(furan-2-yl)-2-methylbenzyl)-1-isobutyryl-6-methyl-4-(phenylsulfonyl)piperazine-2-carboxamide (29)

Step 1: In an analogous manner to 21, (4-bromo-2-methylphenyl)methanamine (27) and 2-(furan-2-yl)-4,4,5,5-tetramethyl-1,3,2-dioxaborolane were used to prepare (4-(furan-2-yl)-2-methylphenyl)methanamine (28). LRMS (ES) (M+H)⁺: observed=188.2. calculated=187.2.

Step 2: In an analogous manner to 19-1, cis-1-isobutyryl-6-methyl-4-(phenylsulfonyl)piperazine-2-carboxylic acid (18) and (4-(furan-2-yl)-2-methylphenyl)methanamine (28) were used to prepare cis-N-(4-(furan-2-yl)-2-methylbenzyl)-1-isobutyryl-6-methyl-4-(phenylsulfonyl)piperazine-2-carboxamide (29) LRMS (ES) (M+H)⁺: observed=524.3. calculated=523.6.

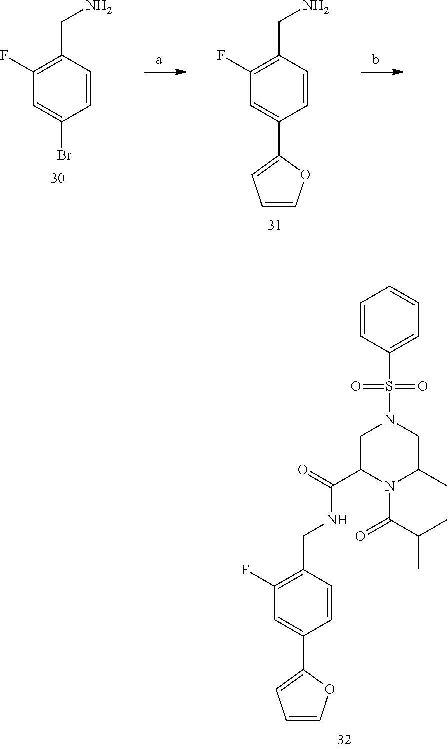 [SMILES: NCc1ccc(Br)cc1F ; NCc1ccc(-c2ccco2)cc1F ; CC(C)C(=O)N1C(C)CN(S(=O)(=O)c2ccccc2)CC1C(=O)NCc1ccc(-c2ccco2)cc1F]

### Preparation of cis-N-(2-fluoro-4-(furan-2-yl)benzyl)-1-isobutyryl-6-methyl-4-(phenylsulfonyl)piperazine-2-carboxamide (32)

Step 1: In an analogous manner to 21, (4-bromo-2-fluorophenyl)methanamine (30) and 2-(furan-2-yl)-4,4,5,5-tetramethyl-1,3,2-dioxaborolane were used to prepare (2-fluoro-4-(furan-2-yl)phenyl)methanamine (31). LRMS (ES) (M+H)⁺: observed=192.1. calculated=191.2.

Step 2: In an analogous manner to 19-1, cis-1-isobutyryl-6-methyl-4-(phenylsulfonyl)piperazine-2-carboxylic acid (18) and (2-fluoro-4-(furan-2-yl)phenyl)methanamine (31) were used to prepare cis-N-(2-fluoro-4-(furan-2-yl)benzyl)-1-isobutyryl-6-methyl-4-(phenylsulfonyl)piperazine-2-carboxamide (32) LRMS (ES) (M+H)⁺: observed=528.3. calculated=527.6.

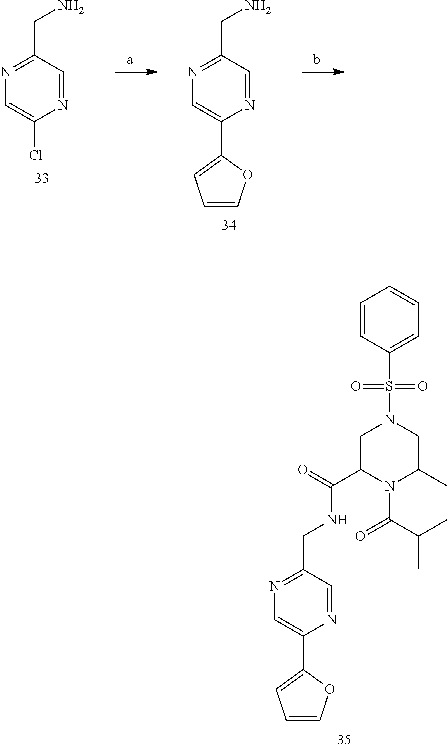 [SMILES: NCc1cnc(Cl)cn1 ; NCc1cnc(-c2ccco2)cn1 ; CC(C)C(=O)N1C(C)CN(S(=O)(=O)c2ccccc2)CC1C(=O)NCc1cnc(-c2ccco2)cn1]

### Preparation of cis-N-((5-(furan-2-yl)pyrazin-2-yl)methyl)-1-isobutyryl-6-methyl-4-(phenylsulfonyl)piperazine-2-carboxamide (35)

Step 1: In an analogous manner to 21, (5-chloropyrazin-2-yl)methanamine (33) and 2-(furan-2-yl)-4,4,5,5-tetramethyl-1,3,2-dioxaborolane were used to prepare (5-(furan-2-yl)pyrazin-2-yl)methanamine (34). LRMS (ES) (M+H)⁺: observed=176.1. calculated=175.1.

Step 2: In an analogous manner to 19-1, cis-1-isobutyryl-6-methyl-4-(phenylsulfonyl)piperazine-2-carboxylic acid (13) and (5-(furan-2-yl)pyrazin-2-yl)methanamine (34) were used to prepare cis-N-((5-(furan-2-yl)pyrazin-2-yl)methyl)-1-isobutyl-6-methyl-4-(phenylsulfonyl)piperazine-2-carboxamide (35) LRMS (ES) (M+H)⁺: observed=512.3. calculated=511.5.

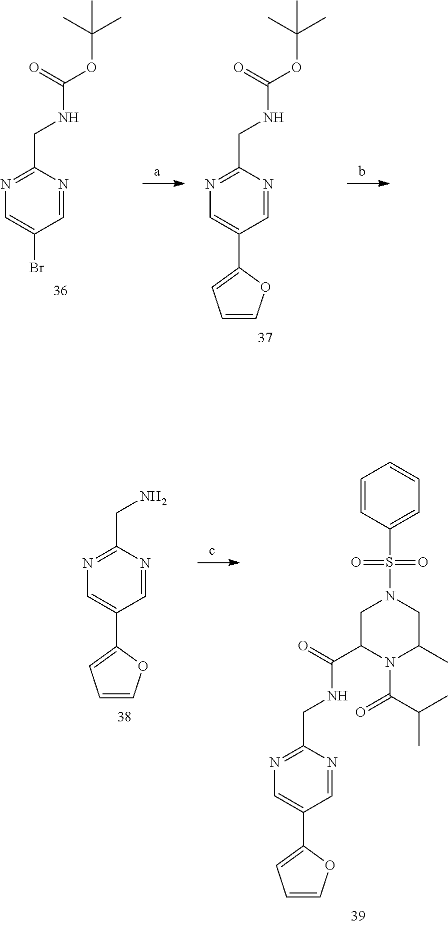 [SMILES: CC(C)(C)OC(=O)NCc1ncc(Br)cn1 ; CC(C)(C)OC(=O)NCc1ncc(-c2ccco2)cn1 ; NCc1ncc(-c2ccco2)cn1 ; CC(C)C(=O)N1C(C)CN(S(=O)(=O)c2ccccc2)CC1C(=O)NCc1ncc(-c2ccco2)cn1]

### Preparation of cis-N-((5-(furan-2-yl)pyrazin-2-yl)methyl)-1-isobutyryl-6-methyl-4-(phenylsulfonyl)piperazine-2-carboxamide (39)

Step 1: In an analogous manner to 21, tert-butyl ((5-bromopyrimidin-2-yl)methyl)carbamate (36) and 2-(furan-2-yl)-4,4,5,5-tetramethyl-1,3,2-dioxaborolane were used to prepare tert-butyl ((5-(furan-2-yl)pyrimidin-2-yl)methyl)carbamate (37). LRMS (ES) (M+H)⁺: observed=276.2. calculated=275.3.

Step 2: In an analogous manner to 3, tert-butyl ((5-(furan-2-yl)pyrimidin-2-yl)methyl)carbamate (37) and HCl were used to prepare (5-(furan-2-yl)pyrimidin-2-yl)methanamine (38). LRMS (ES) (M+H)⁺: observed=176.1. calculated=175.1.

Step 3: In an analogous manner to 19-1, cis-1-isobutyryl-6-methyl-4-(phenylsulfonyl)piperazine-2-carboxylic acid (18) and ((5-(furan-2-yl)pyrimidin-2-yl)methanamine (38) were used to prepare cis-N-((5-(furan-2-yl)pyrazin-2-yl)methyl)-1-isobutyryl-6-methyl-4-(phenylsulfonyl)piperazine-2-carboxamide (39) LRMS (ES) (M+H)⁺: observed=512.3. calculated=511.5.

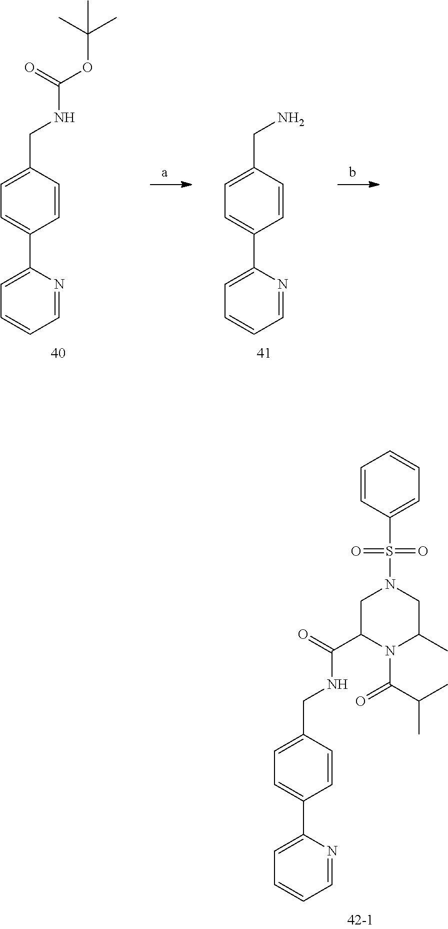 [SMILES: CC(C)(C)OC(=O)NCc1ccc(-c2ccccn2)cc1 ; NCc1ccc(-c2ccccn2)cc1 ; CC(C)C(=O)N1C(C)CN(S(=O)(=O)c2ccccc2)CC1C(=O)NCc1ccc(-c2ccccn2)cc1]

### Preparation of cis-1-isobutyryl-6-methyl-4-(phenylsulfonyl)-N-(4-(pyridin-2-yl)benzyl)piperazine-2-carboxamide (42-1)

Step 1: In an analogous manner to 3, tert-butyl (4-(pyridin-2-yl)benzyl)carbamate (40) and HCl were used to prepare (4-(pyridin-2-yl)phenyl)methanamine HCl (41)

Step 2: In an analogous manner to 19-1, cis-1-isobutyryl-6-methyl-4-(phenylsulfonyl)piperazine-2-carboxylic acid (18) and (4-(pyridin-2-yl)phenyl)methanamine HCl (41) were used to prepare cis-1-isobutyryl-6-methyl-4-(phenylsulfonyl)-N-(4-(pyridin-2-yl)benzyl)piperazine-2-carboxamide (42-1) LRMS (ES) (M+H)⁺: observed=521.5. calculated=520.6.

The following compounds were prepared in an analagous manner to example 42-1.

| | | | Observed | Calculated |
|---|---|---|---|---|
| Example | Compound | Structure | Mass (M + H) | Mass |
| 42-2 | 1-isobutyryl-6- methyl-4- (phenylsulfonyl)- N-(4-(pyridin-3- yl)benzyl)piperazine- 2-carboxamide | 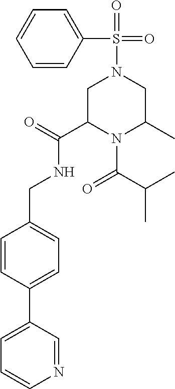 [SMILES: CC(C)C(=O)N1C(C)CN(S(=O)(=O)c2ccccc2)CC1C(=O)NCc1ccc(-c2cccnc2)cc1] | 521.2 | 520.656 |
| 42-3 | 1-isobutyryl-6- methyl-4- (phenylsulfonyl)- N-(4-(pyridin-4- yl)benzyl)piperazine- 2-carboxamide | 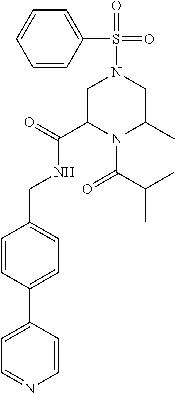 [SMILES: CC(C)C(=O)N1C(C)CN(S(=O)(=O)c2ccccc2)CC1C(=O)NCc1ccc(-c2ccncc2)cc1] | 521.2 | 520.656 |

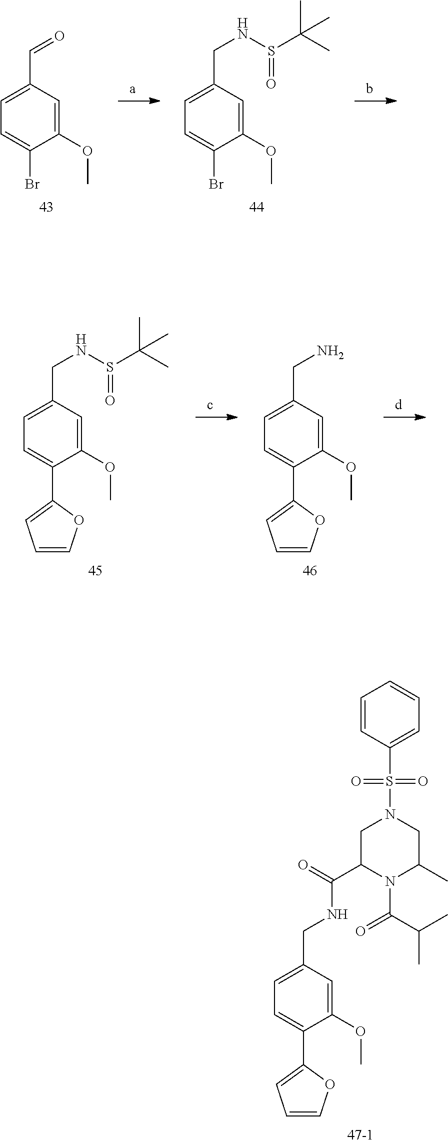 [SMILES: COc1cc(C=O)ccc1Br ; COc1cc(CNS(=O)C(C)(C)C)ccc1Br ; COc1cc(CNS(=O)C(C)(C)C)ccc1-c1ccco1 ; COc1cc(CN)ccc1-c1ccco1 ; COc1cc(CNC(=O)C2CN(S(=O)(=O)c3ccccc3)CC(C)N2C(=O)C(C)C)ccc1-c1ccco1]

### Preparation of cis-N-(4-(furan-2-yl)-3-methoxybenzyl)-1-isobutyryl-6-methyl-4-(phenylsulfonyl)piperazine-2-carboxamide (47-1)

Step 1 Preparation of N-(4-bromo-3-methoxybenzyl)-2-methylpropane-2-sulfinamide To a stirred solution of 4-bromo-3-methoxybenzaldehyde (43, 0.50 g, 2.33 mmol) in THF (4.6 mL) under nitrogen was added Titanium (IV) ethoxide (0.97 mL, 4.65 mmol) and 2-methylpropane-2-sulfinamide (0.37 g, 3.02 mmol) and the resulting mixture was stirred and heated to 55° C. overnight. The reaction was allowed to cool to room temperature and sodium borohydride (0.09 g, 2.32 mmol) was added. After stirring for 1 hr, the reaction was quenched using methanol (0.5 mL). To this was added ethyl acetate and brine, and this mixture was allowed to stir at room temperature. The mixture was filtered through celite, the organic phase extracted with ethyl acetate and concentrated. Flash column purification using a 0-100% ethyl acetate/hexane gradient gave N-(4-bromo-3-methoxybenzyl)-2-methylpropane-2-sulfinamide (44). LRMS (ES) (M+H)⁺: observed=322.2. calculated=320.2.

Step 2: In an analogous manner to 21, N-(4-bromo-3-methoxybenzyl)-2-methylpropane-2-sulfinamide (44) and 2-(furan-2-yl)-4,4,5,5-tetramethyl-1,3,2-dioxaborolane were used to prepare N-(4-(furan-2-yl)-3-methoxybenzyl)-2-methylpropane-2-sulfinamide (45). LRMS (ES) (M+H)⁺: observed=308.3. calculated=307.4.

Step 3: In an analogous manner to 3, N-(4-(furan-2-yl)-3-methoxybenzyl)-2-methylpropane-2-sulfinamide (45) and HCl were used to prepare (4-(furan-2-yl)-3-methoxyphenyl)methanamine HCl (46)

Step 4: In an analogous manner to 19-1, cis-N-isobutyryl-6-methyl-4-(phenylsulfonyl) piperazine-2-carboxylic acid (18) and (4-(furan-2-yl)-3-methoxyphenyl)methanamine HCl (45) were used to prepare cis-N-(4-(furan-2-yl)-3-methoxybenzyl)-1-isobutyryl-6-methyl-4-(phenylsulfonyl)piperazine-2-carboxamide (47-1) LRMS (ES) (M+H)⁺: observed=540.4. calculated=539.6.

The following compounds were prepared in an analagous manner to example 47-1.

| | | | Observed | |
|---|---|---|---|---|
| | | | Mass | Calculated |
| Example | Compound | Structure | (M + H) | Mass |
| 47-2 | cis-N-(3-cyano-4- (furan-2-yl)benzyl)- 1-isobutyryl-6- methyl-4- (phenylsulfonyl) piperazine- 2-carboxamide | 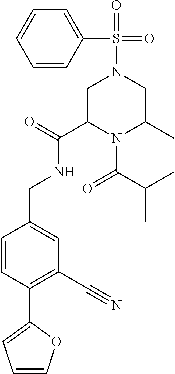 [SMILES: CC(C)C(=O)N1C(C)CN(S(=O)(=O)c2ccccc2)CC1C(=O)NCc1ccc(-c2ccco2)c(C#N)c1] | 535.4 | 534.639 |
| 47-3 | cis-N-(3,5-difluoro- 4-(furan-2- yl)benzyl)-1- isobutyryl-6- methyl-4- (phenylsulfonyl) piperazine- 2-carboxamide | 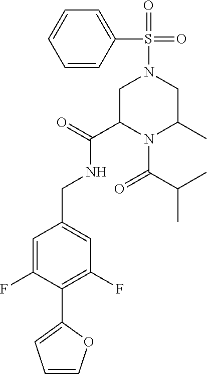 [SMILES: CC(C)C(=O)N1C(C)CN(S(=O)(=O)c2ccccc2)CC1C(=O)NCc1cc(F)c(-c2ccco2)c(F)c1] | 546.4 | 545.61 |
| 47-4 | cis-N-(4-(furan-2- yl)-2- methoxybenzyl)-1- isobutyryl-6- methyl-4- (phenylsulfonyl) piperazine- 2-carboxamide | 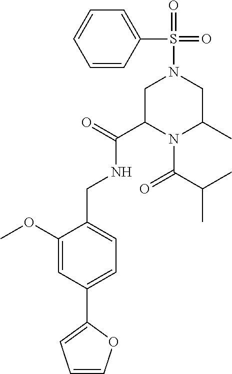 | 540.4 | 539.656 |
| 47-5 | cis-N-(5-fluoro-4- (furan-2-yl)-2- methylbenzyl)-1- isobutyryl-6- methyl-4- (phenylsulfonyl) piperazine- 2-carboxamide | 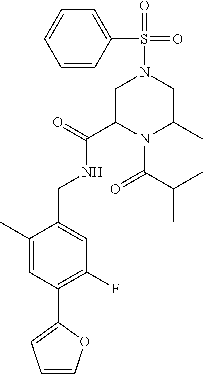 | 542.4 | 541.647 |
| 47-6 | cis-N-(2-ethyl-4- (furan-2-yl)benzyl)- 1-isobutyryl-6- methyl-4- (phenylsulfonyl) piperazine- 2-carboxamide | 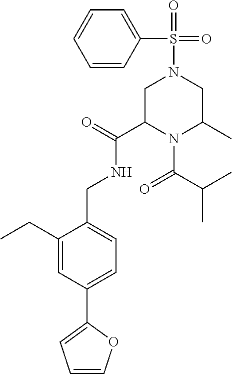 [SMILES: CCc1cc(-c2ccco2)ccc1CNC(=O)C1CN(S(=O)(=O)c2ccccc2)CC(C)N1C(=O)C(C)C] | 538.5 | 537.683 |

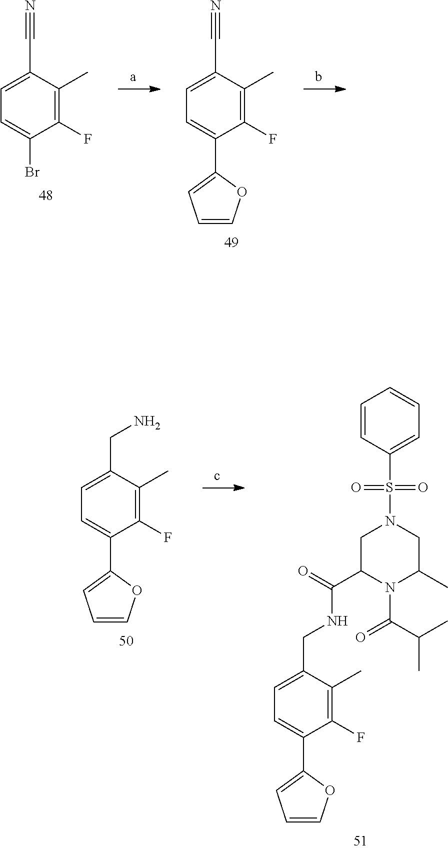 [SMILES: Cc1c(C#N)ccc(Br)c1F ; Cc1c(C#N)ccc(-c2ccco2)c1F ; Cc1c(CN)ccc(-c2ccco2)c1F ; Cc1c(CNC(=O)C2CN(S(=O)(=O)c3ccccc3)CC(C)N2C(=O)C(C)C)ccc(-c2ccco2)c1F]

### Preparation of cis-N-(3-fluoro-4-(furan-2-yl)-2-methylbenzyl)-1-isobutyryl-6-methyl-4-(phenylsulfonyl)piperazine-2-carboxamide (51)

Step 1: In an analogous manner to 21, 4-bromo-3-fluoro-2-methylbenzonitrile (48) and 2-(furan-2-yl)-4,4,5,5-tetramethyl-1,3,2-dioxaborolane were used to prepare 3-fluoro-4-(furan-2-yl)-2-methylbenzonitrile (49, 64%). LRMS (ES) (M+H)⁺: observed=N/D. calculated=201.2.

Step 2: Preparation of (3-fluoro-4-(furan-2-yl)-2-methylphenyl)methanamine (50) To a stirred solution of 3-fluoro-4-(furan-2-yl)-2-methylbenzonitrile (49, 0.24 g, 1.20 mmol) in methanol (4.8 mL) chilled in an ice bath to 0° C. was added Nickel (II) Chloride hexahydrate (0.28 g, 1.20 mmol). To this was added sodium borohydride (0.14 g, 3.61 mmol) portionwise over 15 minutes. The resulting mixture was stirred for 30 minutes at 0° C. and concentrated. The crude material was partitioned between Ethyl acetate and saturated sodium bicarbonate solution. Both phases were sonicated and filtered through celite. The organic phase was concentrated and flash column purification using a 0-50% isopropanol/DCM gradient gave (3-fluoro-4-(furan-2-yl)-2-methylphenyl)methanamine (50). LRMS (ES) (M+H)⁺: observed=206.2. calculated=205.2.

Step 3: In an analogous manner to 19-1, cis-1-isobutyryl-6-methyl-4-(phenylsulfonyl)piperazine-2-carboxylic acid (18) and (3-fluoro-4-(furan-2-yl)-2-methylphenyl)methanamine (50) were used to prepare cis-N-(3-fluoro-4-(furan-2-yl)-2-methylbenzyl)-1-isobutyryl-6-methyl-4-(phenylsulfonyl)piperazine-2-carboxamide (51) LRMS (ES) (M+H)⁺: observed=542.4. calculated=541.6.

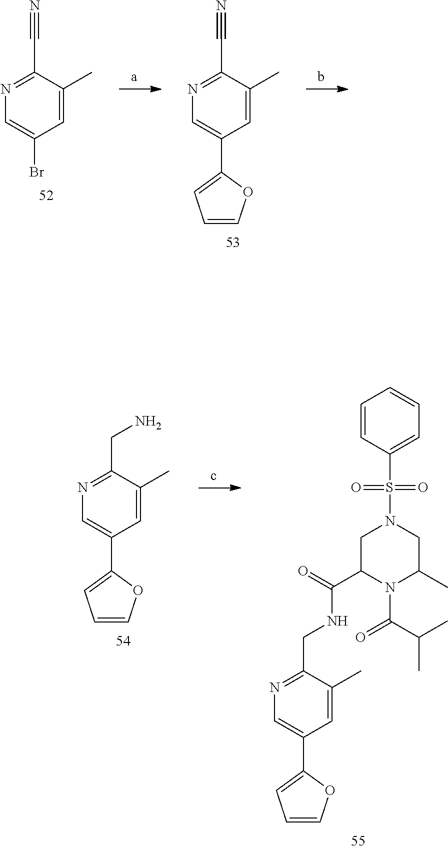

### Preparation of cis-N-((5-(furan-2-yl)-3-methylpyridin-2-yl)methyl)-1-isobutyryl-6-methyl-4-(phenylsulfonyl)piperazine-2-carboxamide (55)

Step 1: In an analogous manner to 21, 5-bromo-3-methylpicolinonitrile (52) and 2-(furan-2-yl)-4,4,5,5-tetramethyl-1,3,2-dioxaborolane were used to prepare 5-(furan-2-yl)-3-methylpicolinonitrile (53, 52%). LRMS (ES) (M+H)⁺: observed=185.1. calculated=184.1.

Step 2: In an analogous manner to 50, 5-(furan-2-yl)-3-methylpicolinonitrile (53) was used to prepare (5-(furan-2-yl)-3-methylpyridin-2-yl)methanamine (54). LRMS (ES) (M+H)⁺: observed=189.1. calculated=188.2.

Step 3: In an analogous manner to 19-1, cis-1-isobutyryl-6-methyl-4-(phenylsulfonyl)piperazine-2-carboxylic acid (18) and (5-(furan-2-yl)-3-methylpyridin-2-yl)methanamine (54) were used to prepare cis-N-((5-(furan-2-yl)-3-methylpyridin-2-yl)methyl)-1-isobutyryl-6-methyl-4-(phenylsulfonyl)piperazine-2-carboxamide (55). LRMS (ES) (M+H)⁺: observed=525.4. calculated=524.6.

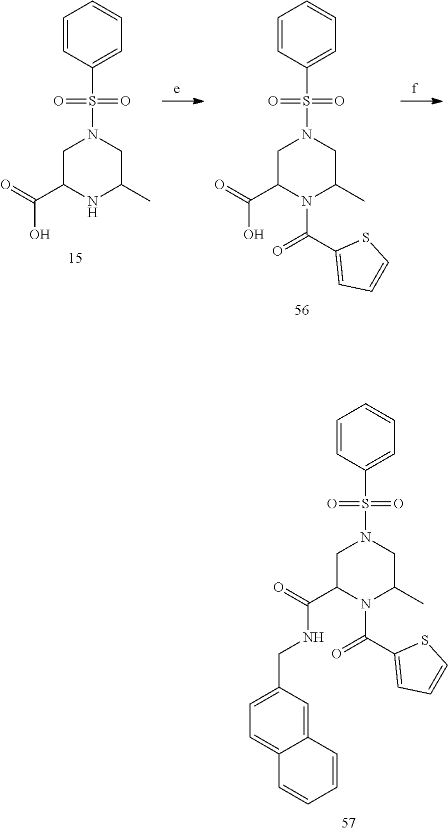 [SMILES: CC1CN(S(=O)(=O)c2ccccc2)CC(C(=O)O)N1 ; CC1CN(S(=O)(=O)c2ccccc2)CC(C(=O)O)N1C(=O)c1cccs1 ; CC1CN(S(=O)(=O)c2ccccc2)CC(C(=O)NCc2ccc3ccccc3c2)N1C(=O)c1cccs1]

### Preparation of cis-6-methyl-N-(naphthalen-2-ylmethyl)-4-(phenylsulfonyl)-1-(thiophene-2-carbonyl)piperazine-2-carboxamide (57)

Step 1: Preparation of 6-methyl-4-(phenylsulfonyl)-1-(thiophene-2-carbonyl)piperazine-2-carboxylic acid (56) To a solution of cis-6-methyl-4-(phenylsulfonyl)piperazine-2-carboxylic acid (15, 0.2 g, 0.70 mmol) in DCM (5.0 ml) was added triethylamine (0.214 g, 2.11 mmol), thiophene-2-carbonyl chloride (0.11 g, 0.77 mmol). Then the reaction mixture was stirred at room temperature for 2 hours. LCMS show that the reaction was completed. After concentration, the residue was purified by PRE-HPLC (TFA) to give 6-methyl-4-(phenylsulfonyl)-1-(thiophene-2-carbonyl)piperazine-2-carboxylic acid (56). LRMS (ES) (M+H)⁺: observed=395.0. calculated=394.5.

Step 2: In an analogous manner to 19-1, 6-methyl-4-(phenylsulfonyl)-1-(thiophene-2-carbonyl) piperazine-2-carboxylic acid (56) and naphthalen-2-ylmethanamine were used to prepare cis-6-methyl-N-(naphthalen-2-ylmethyl)-4-(phenylsulfonyl)-1-(thiophene-2-carbonyl)piperazine-2-carboxamide (57) LRMS (ES) (M+H)⁺: observed=534.0. calculated=533.7.

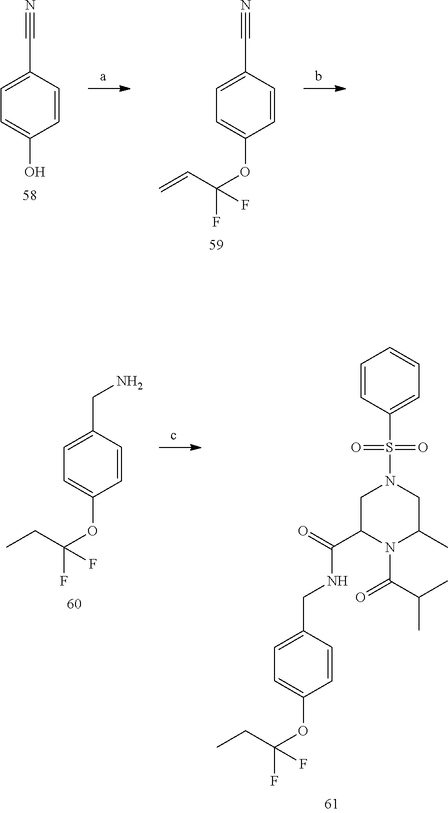 [SMILES: N#Cc1ccc(O)cc1 ; C=CC(F)(F)Oc1ccc(C#N)cc1 ; CCC(F)(F)Oc1ccc(CN)cc1 ; CCC(F)(F)Oc1ccc(CNC(=O)C2CN(S(=O)(=O)c3ccccc3)CC(C)N2C(=O)C(C)C)cc1]

### Preparation of cis-N-(4-(1,1-difluoropropoxy)benzyl)-1-isobutyryl-6-methyl-4-(phenylsulfonyl)piperazine-2-carboxamide (61)

Step 1: Preparation of 4-((1,1-difluoroallyl)oxy)benzonitrile (59) To a solution of 4-hydroxybenzonitrile (58, 0.72 g, 6.04 mmol) in DMF (10.0 ml) were added K₂CO₃ (1.67 g, 12.09 mmol) and 3-bromo-3,3-difluoroprop-1-ene (1.90 g, 12.09 mmol), then the solution was stirred at 50° C. for 5 hours. The reaction mixture was poured into water (10 mL), extracted with EtOAc (20 mL×3). The organic layer was washed with brine (20 mL), dried over Na₂SO₄ and filtered. After filtration was concentrated to give crude product, the crude product was purified by flash silica gel chromatography (ISCO®; 12 g SepaFlash® Silica Flash Column, eluent of 1.6% ethyl acetate/pet. ether gradient @30 mL/min) to give 4-((1,1-difluoroallyl)oxy)benzonitrile (59).

Step 2: Preparation of (4-(1,1-difluoropropoxy)phenyl)methanamine (60) To a solution of nickel (0.50 g, 8.52 mmol) in MeOH (10.0 ml) and NH₃. H₂O (10.0 ml) was added compound 2 (0.30 g, 1.54 mmol), then the solution was stirred at 20° C. for 5 hours under H2 (15 psi). The reaction mixture was filtered and concentrated to give crude (4-(1,1-difluoropropoxy)phenyl) methanamine (60).

Step 3: In an analogous manner to 19-1, cis-1-isobutyryl-6-methyl-4-(phenylsulfonyl) piperazine-2-carboxylic acid (18) and (4-(1,1-difluoropropoxy)phenyl)methanamine (60) were used to prepare cis-N-(4-(1,1-difluoropropoxy)benzyl)-1-isobutyryl-6-methyl-4-(phenylsulfonyl) piperazine-2-carboxamide (61). LRMS (ES) (M+H)⁺: observed=538.0. calculated=537.6.

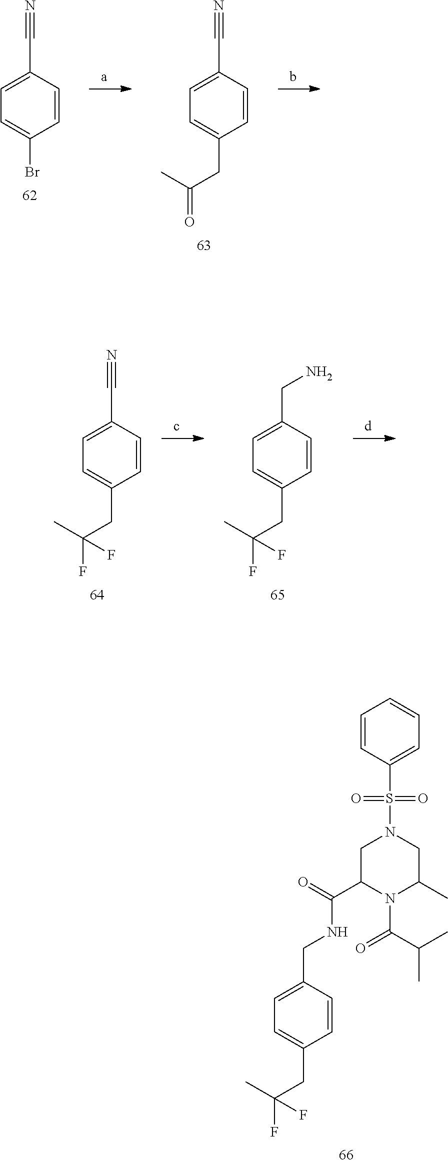 [SMILES: N#Cc1ccc(Br)cc1 ; CC(=O)Cc1ccc(C#N)cc1 ; CC(F)(F)Cc1ccc(C#N)cc1 ; CC(F)(F)Cc1ccc(CN)cc1 ; CC(C)C(=O)N1C(C)CN(S(=O)(=O)c2ccccc2)CC1C(=O)NCc1ccc(CC(C)(F)F)cc1]

### Preparation of cis-N-(4-(1,1-difluoropropoxy)benzyl)-1-isobutyryl-6-methyl-4-(phenylsulfonyl)piperazine-2-carboxamide (66)

Step 1: Preparation of 4-(2-oxopropyl)benzonitrile (63) To a solution of 4-bromobenzonitrile (5.0 g, 27.50 mmol) in anhydrous toluene (50.0 ml) was added prop-1-en-2-yl acetate (5.50 g, 54.90 mmol), tri-o-tolylphosphine (0.84 g, 2.75 mmol), tri-N-butyltin methoxide (9.25 g, 28.8 mmol), Pd(OAc)₂ (0.31 g, 1.37 mmol). The resulting mixture was stirred at 105° C. under N₂ for 12 hours. The reaction mixture was added to aq. KF (100 ml) and stirred for 30 minutes. Then the reaction mixture was filtered and extracted with ethyl acetate (50 mL×3). The organic layer was washed with brine (50 mL×3), and dried over Na₂SO₄. After filtration and concentration, the crude was purified by flash column chromatography (SiO₂, PE:EA=3:1) to give 4-(2-oxopropyl)benzonitrile (63). LRMS (ES) (M+H)⁺: observed=159.8. calculated=159.1.

Step 2: Preparation of 4-(2,2-difluoropropyl)benzonitrile (64) To a solution of 4-(2-oxopropyl)benzonitrile (63, 0.160 g, 1.01 mmol) in anhydrous DCM (5 ml) was added DAST (0.66 ml, 5.03 mmol) at 10° C. The resulting mixture was stirred at 10° C. under N₂ for 12 hours. Then the reaction mixture was poured into water (10 mL) and extracted with DCM (10 mL×3). The organic layer was washed with brine (10 mL×3), dried over Na₂SO₄. After filtration and concentration, the residue was purified by pre-TLC (SiO₂, PE:EA=5:1) to give 4-(2,2-difluoropropyl)benzonitrile (64).

Step 3: Preparation of (4-(2,2-difluoropropyl)phenyl)methanamine (65) To a solution of 4-(2,2-difluoropropyl)benzonitrile (64, 0.14 g, 0.77 mmol) in anhydrous THF (5 ml) was added BH3 (2.32 ml, 2.32 mmol, 1M). Then the resulting mixture was stirred at 75° C. under N₂ for 5 hours. The reaction was quenched with 3M HCl (3 ml) and stirred for 12 h. The reaction mixture was neutralized with aq. NaHCO₃ to pH=8, and extracted with EA (20 mL×3). The organic layer was washed with brine (20 mL×3), dried over Na₂SO₄. After filtration and concentration, the crude product (4-(2,2-difluoropropyl)phenyl)methanamine (65) was obtained. LRMS (ES) (M+H)⁺: observed=169.0 [M-17+H+]. calculated=185.2.

Step 4: In an analogous manner to 19-1, cis-1-isobutyryl-6-methyl-4-(phenylsulfonyl) piperazine-2-carboxylic acid (18) and (4-(2,2-difluoropropyl)phenyl)methanamine (65) were used to prepare cis-N-(4-(1,1-difluoropropoxy)benzyl)-1-isobutyryl-6-methyl-4-(phenylsulfonyl)piperazine-2-carboxamide (66).

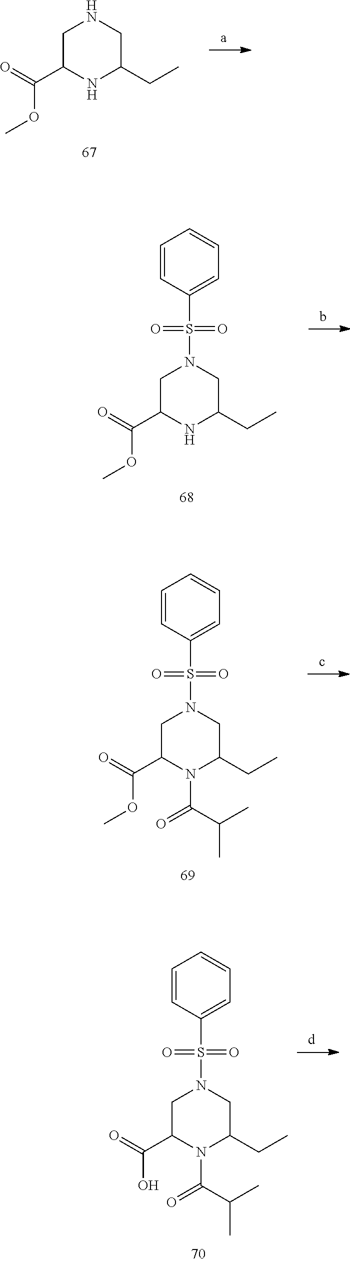

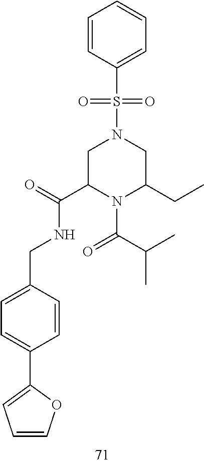 [SMILES: CCC1CN(S(=O)(=O)c2ccccc2)CC(C(=O)NCc2ccc(-c3ccco3)cc2)N1C(=O)C(C)C]

### Preparation of cis-6-ethyl-N-(4-(furan-2-yl)benzyl)-1-isobutyryl-4-(phenylsulfonyl)piperazine-2-carboxamide (71)

Step 1: Preparation of cis-methyl 6-ethyl-4-(phenylsulfonyl)piperazine-2-carboxylate (68) To a solution of cis-methyl 6-ethylpiperazine-2-carboxylate (67, 0.40 g, 2.32 mmol) in DCM (5 mL) were added benzenesulfonyl chloride (0.37 g, 2.09 mmol) and TEA (0.65 mL, 4.65 mmol). The mixture was stirred at 25° C. for 16 h. To the mixture was added water (20 mL), which was extracted with DCM:MeOH=10:1 (20 mL×3). The combined organic layers were washed with brine (20 mL), dried over MgSO₄ and filtered. The filtrate was concentrated in vacuo to give the crude, which was purified by prep-TLC (SiO₂, EtOAc) to give cis-methyl 6-ethyl-4-(phenylsulfonyl)piperazine-2-carboxylate (68). LRMS (ES) (M+H)⁺: observed=313.1 [M-17+H+]. calculated=312.4.

Step 2: In an analogous manner to 17, cis-methyl 6-ethyl-4-(phenylsulfonyl)piperazine-2-carboxylate (68) and isobutyryl chloride were used to prepare cis-methyl 6-ethyl-1-isobutyryl-4-(phenylsulfonyl)piperazine-2-carboxylate (69).

Step 3: In an analogous manner to 18, cis-methyl 6-ethyl-1-isobutyryl-4-(phenylsulfonyl)piperazine-2-carboxylate (69) and potassium trimethylsilanolate were used to prepare cis-6-ethyl-1-isobutyryl-4-(phenylsulfonyl)piperazine-2-carboxylic acid (70).

Step 4: Preparation of cis-6-ethyl-N-(4-(furan-2-yl)benzyl)-1-isobutyryl-4-(phenylsulfonyl)piperazine-2-carboxamide (71) To a solution of cis-6-ethyl-1-isobutyryl-4-(phenylsulfonyl)piperazine-2-carboxylic acid (70, 0.03 g, 0.081 mmol) in DMF (1 mL) were added DIPEA (0.04 mL, 0.24 mmol), EDC (0.023 g, 0.122 mmol), HOBT (0.018 g, 0.122 mmol) and (4-(furan-2-yl)phenyl)methanamine (0.016 mg, 0.09 mmol). The mixture was degassed and backfilled with N2 for three times. The reaction mixture was stirred at 25° C. for 5 h. The mixture was diluted with EtAOc (10 mL), which was washed with water (10 mL×2) and brine (10 mL×2), dried over MgSO4 and filtered. The filtrate was concentrated in vacuo to give the residue, which was purified by prep-HPLC (TFA) to give cis-6-ethyl-N-(4-(furan-2-yl)benzyl)-1-isobutyryl-4-(phenylsulfonyl)piperazine-2-carboxamide (71). LRMS (ES) (M+H)⁺: observed=524.0. calculated=523.6.

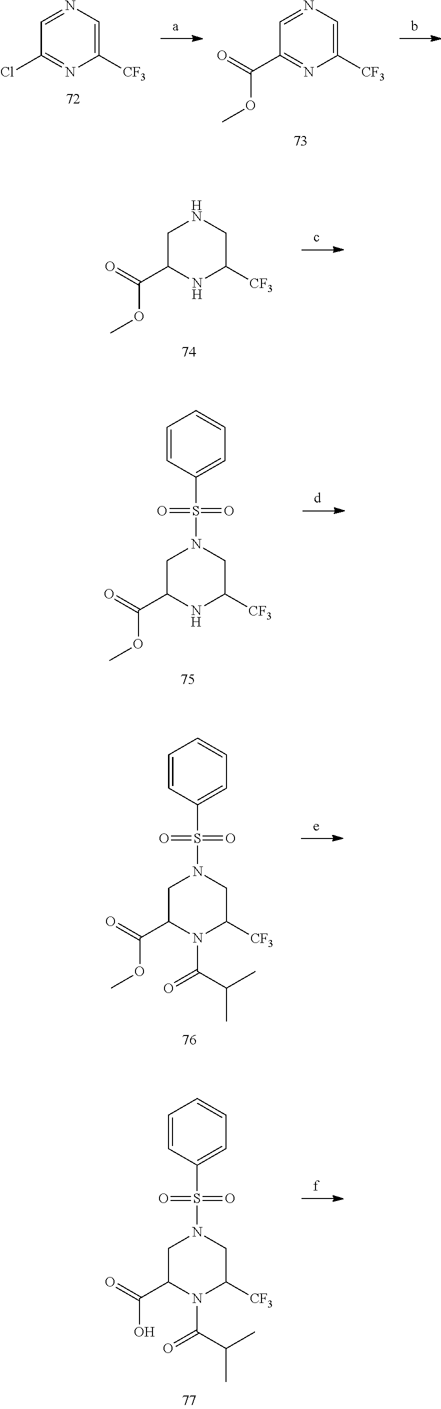 [SMILES: FC(F)(F)c1cncc(Cl)n1 ; COC(=O)c1cncc(C(F)(F)F)n1 ; COC(=O)C1CNCC(C(F)(F)F)N1 ; COC(=O)C1CN(S(=O)(=O)c2ccccc2)CC(C(F)(F)F)N1 ; COC(=O)C1CN(S(=O)(=O)c2ccccc2)CC(C(F)(F)F)N1C(=O)C(C)C ; CC(C)C(=O)N1C(C(=O)O)CN(S(=O)(=O)c2ccccc2)CC1C(F)(F)F]

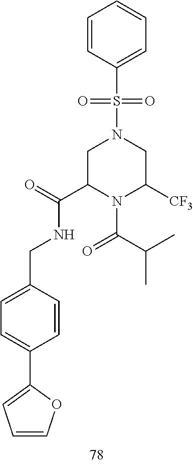 [SMILES: CC(C)C(=O)N1C(C(=O)NCc2ccc(-c3ccco3)cc2)CN(S(=O)(=O)c2ccccc2)CC1C(F)(F)F]

### Preparation of cis-N-(4-(furan-2-yl)benzyl)-1-isobutyryl-4-(phenylsulfonyl)-6-(trifluoromethyl)piperazine-2-carboxamide (78)

Step 1: Preparation of methyl 6-(trifluoromethyl)pyrazine-2-carboxylate (73) A mixture of 2-chloro-6-(trifluoromethyl)pyrazine (72, 0.19 g, 1.04 mmol), DPPF (0.035 g, 0.06 mmol), Pd(OAc)2 (0.018 g, 0.08 mmol) and Et₃N (0.22 mL, 1.56 mmol) in MeOH (6 mL) was stirred at 70° C. under CO (excess) (50 psi) for 24 h. Then the solution was filtered and concentrated in vacuo. The mixture was purified by prep-TLC (SiO₂, PE:EtOAc=5:1) to give methyl 6-(trifluoromethyl)pyrazine-2-carboxylate (73). LRMS (ES) (M+H)⁺: observed=207.0. calculated=206.1.

Step 2: Preparation of cis-methyl 6-(trifluoromethyl)piperazine-2-carboxylate (74) To a mixture of methyl 6-(trifluoromethyl)pyrazine-2-carboxylate (73, 0.12 g, 0.58 mmol) in AcOH (5 mL) were added Pd/C (0.069 g, 0.06 mmol) (10%). The mixture was stirred at 25° C. under H2 (excess) (50 psi) for 16 h. Then the solution was filtered and concentrated in vacuo to give cis-methyl 6-(trifluoromethyl)piperazine-2-carboxylate (74). LRMS (ES) (M+H)⁺: observed=213.1. calculated=212.2.

Step 3: In an analogous manner to 68, cis-methyl 6-(trifluoromethyl)piperazine-2-carboxylate (74) and benzenesulfonyl chloride were used to prepare cis-methyl 4-(phenylsulfonyl)-6-(trifluoromethyl)piperazine-2-carboxylate (75).

Step 4: In an analogous manner to 17, cis-methyl 4-(phenylsulfonyl)-6-(trifluoromethyl) piperazine-2-carboxylate (75) and isobutyryl chloride were used to prepare cis-methyl 1-isobutyryl-4-(phenylsulfonyl)-6-(trifluoromethyl)piperazine-2-carboxylate (76).

Step 5: In an analogous manner to 18, cis-methyl 1-isobutyryl-4-(phenylsulfonyl)-6-(trifluoromethyl)piperazine-2-carboxylate (76) and potassium trimethylsilanolate were used to prepare cis-1-isobutyryl-4-(phenylsulfonyl)-6-(trifluoromethyl)piperazine-2-carboxylic acid (77).

Step 6: In an analogous manner to 71, cis-1-isobutyryl-4-(phenylsulfonyl)-6-(trifluoromethyl)piperazine-2-carboxylic acid (77) and (4-(furan-2-yl)phenyl)methanamine were used to prepare cis-N-(4-(furan-2-yl)benzyl)-1-isobutyryl-4-(phenylsulfonyl)-6-(trifluoromethyl)piperazine-2-carboxamide (78).

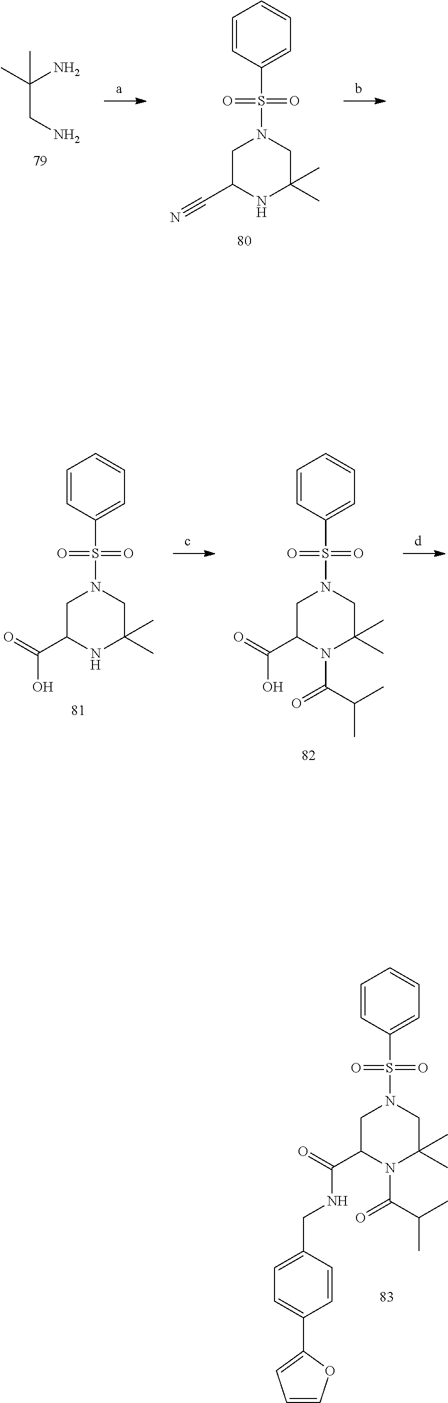

### Preparation of N-(4-(furan-2-yl)benzyl)-1-isobutyryl-6,6-dimethyl-4-(phenylsulfonyl)piperazine-2-carboxamide (83)

Step 1: Preparation of 6,6-dimethyl-4-(phenylsulfonyl)piperazine-2-carbonitrile (80) To a solution of 2-methylpropane-1,2-diamine (79, 4.98 g, 56.45 mmol) in THF (100 ml) was 2-chloroacrylonitrile (3.80 g, 43.43 mmol) and the solution was stirred at 50° C. for 4 hours. Then Et3N (8.79 g, 86.85 mmol) and benzenesulfonyl chloride (9.97 g, 56.45 mmol) were added to the above solution at 20° C. The mixture was stirred at 20° C. for 5 hours. The reaction mixture was poured into water (100 mL), extracted with EtOAc (50 mL×3). The organic layer was washed with brine (100 mL), dried over Na2SO4. After filtration was concentrated, the crude product was purified by flash silica gel chromatography (ISCO®; 80 g SepaFlash® Silica Flash Column, eluent of 25% ethyl acetate/pet. ether gradient @60 mL/min) to give Preparation of 6,6-dimethyl-4-(phenylsulfonyl)piperazine-2-carbonitrile (80).

Step 2: Preparation of 6,6-dimethyl-4-(phenylsulfonyl)piperazine-2-carboxylic acid (81) To a solution of 6,6-dimethyl-4-(phenylsulfonyl)piperazine-2-carbonitrile (80, 1.00 g, 3.58 mmol) in DMSO (10 ml) were added KOH (0.20 g, 3.58 mmol) and H2O2 (2 ml), then the solution was stirred at 20° C. for 24 hours. The reaction mixture was poured into water (20 mL), extracted with EtOAc (30 mL×5). The desired product in water phase was purified by pre-HPLC (TFA) to give 6,6-dimethyl-4-(phenylsulfonyl)piperazine-2-carboxylic acid (81).

Step 3: Preparation of 1-isobutyryl-6,6-dimethyl-4-(phenylsulfonyl)piperazine-2-carboxylic acid (82) To a solution of 6,6-dimethyl-4-(phenylsulfonyl)piperazine-2-carboxylic acid (81, 0.30 g, 1.01 mmol) in Pyridine (2 ml) were added isobutyryl chloride (0.54 g, 5.03 mmol) and DMAP (0.012 g, 0.10 mmol), then the solution was stirred at 20° C. for 2 hours. The reaction mixture was poured into water (10 mL), extracted with EtOAc (20 mL×3). The organic layer was washed with brine (30 mL), dried over Na2SO4 and filtered. After filtration was concentrated to give crude product, the crude product was purified by pre-HPLC (TFA) to give 1-isobutyryl-6,6-dimethyl-4-(phenylsulfonyl)piperazine-2-carboxylic acid (82).

Step 4: In an analogous manner to 19-1, 1-isobutyryl-6,6-dimethyl-4-(phenylsulfonyl) piperazine-2-carboxylic acid (82) and (4-(furan-2-yl)phenyl)methanamine were used to prepare N-(4-(furan-2-yl)benzyl)-1-isobutyryl-6,6-dimethyl-4-(phenylsulfonyl)piperazine-2-carboxamide (83).

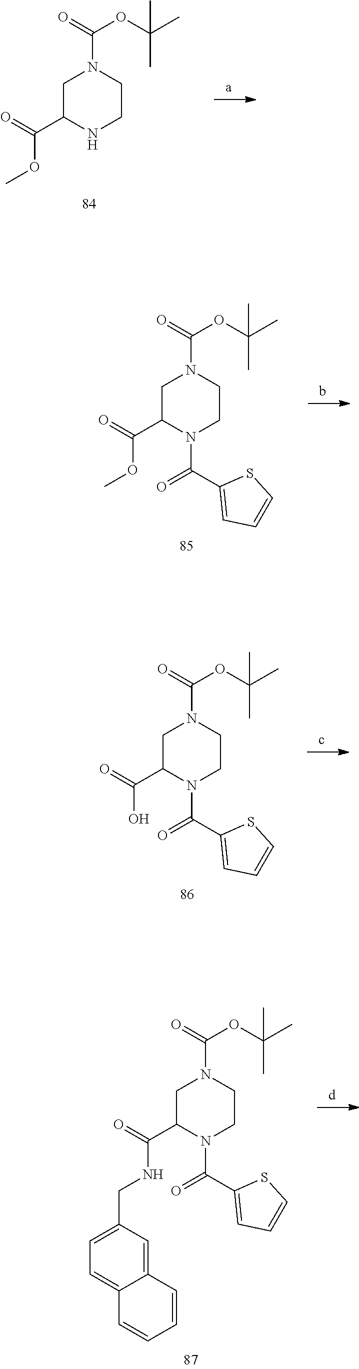 [SMILES: COC(=O)C1CN(C(=O)OC(C)(C)C)CCN1 ; COC(=O)C1CN(C(=O)OC(C)(C)C)CCN1C(=O)c1cccs1 ; CC(C)(C)OC(=O)N1CCN(C(=O)c2cccs2)C(C(=O)O)C1 ; CC(C)(C)OC(=O)N1CCN(C(=O)c2cccs2)C(C(=O)NCc2ccc3ccccc3c2)C1]

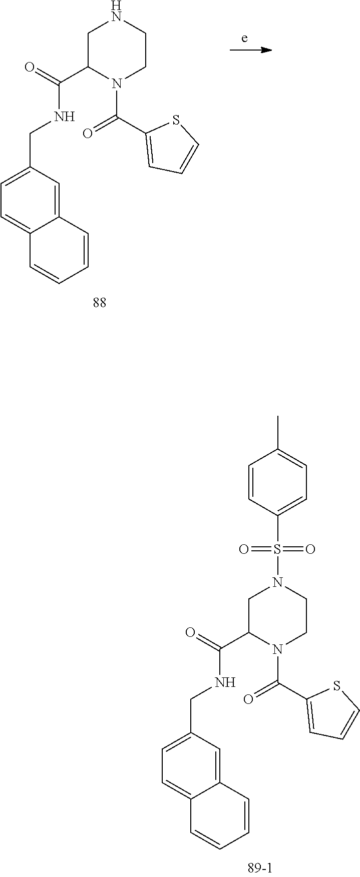 [SMILES: O=C(NCc1ccc2ccccc2c1)C1CNCCN1C(=O)c1cccs1 ; Cc1ccc(S(=O)(=O)N2CCN(C(=O)c3cccs3)C(C(=O)NCc3ccc4ccccc4c3)C2)cc1]

### Preparation of N-(naphthalen-2-ylmethyl)-1-(thiophene-2-carbonyl)-4-tosylpiperazine-2-carboxamide (89-1)

Step 1: Preparation of 1-(tert-butyl) 3-methyl 4-(thiophene-2-carbonyl)piperazine-1,3-dicarboxylate (85). To a stirred solution of 1-(tert-butyl) 3-methyl piperazine-1,3-dicarboxylate (84, 1.00 g, 4.09 mmol) and thiophene-2-carboxylic acid (0.55 g, 4.30 mmol) in DCM (11.7 mL) was added DMAP (1.00 g, 8.19 mmol) and EDC (0.94 g, 4.91 mmol). The resulting mixture was stirred overnight, then washed with 1N HCl solution and concentrated. Flash column purification using a 0-50% ethyl acetate/hexane gradient gave 1-(tert-butyl) 3-methyl 4-(thiophene-2-carbonyl)piperazine-1,3-dicarboxylate (85). LRMS (ES) (M+H)⁺: observed=355.3. calculated=354.2.

Step 2: Preparation of 4-(tert-butoxycarbonyl)-1-(thiophene-2-carbonyl)piperazine-2-carboxylic acid (86). To a stirred solution of 1-(tert-butyl) 3-methyl 4-(thiophene-2-carbonyl)piperazine-1,3-dicarboxylate (85, 0.45 g 1.25 mmol) in a mixture of THF (5.2 mL) and methanol (5.2 mL) was added lithium hydroxide solution (1M, 2.51 mL, 2.51 mmol). The resulting mixture was heated to 55° C. overnight. The reaction was concentrated and partitioned between EtOAc and 1N HCl solution. The organic phase was concentrated to give 4-(tert-butoxycarbonyl)-1-(thiophene-2-carbonyl)piperazine-2-carboxylic acid (86). LRMS (ES) (M+H)⁺: observed=341.2. calculated=340.4.

Step 3: Preparation of tert-butyl 3-((naphthalen-2-ylmethyl)carbamoyl)-4-(thiophene-2-carbonyl)piperazine-1-carboxylate (87). To a stirred solution of 4-(tert-butoxycarbonyl)-1-(thiophene-2-carbonyl)piperazine-2-carboxylic acid (86, 0.57 g, 1.68 mmol) and naphthalen-2-ylmethanamine (0.29 g, 2.02 mmol) in DCM (4.8 mL) was added DMAP (0.41 g, 3.37 mmol) and EDC (0.39 g, 4.91 mmol). The resulting mixture was stirred overnight, then washed with 1N HCl solution and concentrated. Flash column purification using a 0-60% ethyl acetate/hexane gradient gave 3-((naphthalen-2-ylmethyl)carbamoyl)-4-(thiophene-2-carbonyl)piperazine-1-carboxylate (87). LRMS (ES) (M+H)⁺: observed=480.4. calculated=479.6.

Step 4: Preparation of N-(naphthalen-2-ylmethyl)-1-(thiophene-2-carbonyl)piperazine-2-carboxamide (88) A stirred solution of 3-((naphthalen-2-ylmethyl)carbamoyl)-4-(thiophene-2-carbonyl)piperazine-1-carboxylate (87, 0.69 g, 1.44 mmol) in Ethyl Acetate (7 mL) was chilled in an ice bath to 0° C. To this solution was bubbled in HCl gas. The solution was stirred 75 min and concentrated to give Preparation of N-(naphthalen-2-ylmethyl)-1-(thiophene-2-carbonyl)piperazine-2-carboxamide (88) as an HCl salt. LRMS (ES) (M+H)⁺: observed=380.3. calculated=379.3.

Step 5: Preparation of N-(naphthalen-2-ylmethyl)-1-(thiophene-2-carbonyl)-4-tosylpiperazine-2-carboxamide (89-1) To a stirred solution of N-(naphthalen-2-ylmethyl)-1-(thiophene-2-carbonyl)piperazine-2-carboxamide (88, 0.02 g, 0.048 mmol) in DCM (0.25 mL) was added pyridine (0.02 mL, 0.24 mmol) and 4-methylbenzenesulfonyl chloride (6.8 mg, 0.06 mmol) at room temperature. The resulting solution was stirred and concentrated. The crude mixture was purified by reverse phase purification to give N-(naphthalen-2-ylmethyl)-1-(thiophene-2-carbonyl)-4-tosylpiperazine-2-carboxamide (89-1) LRMS (ES) (M+H)⁺: observed=534.1. calculated=533.6.

The following compounds were prepared in an analagous manner to example 89-1

| | | | Calculated | Observed |
|---|---|---|---|---|
| Example | Structure | Compound | Mass | Mass (M + H) |
| 89-2 | 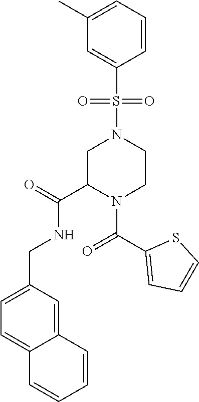 [SMILES: Cc1cccc(S(=O)(=O)N2CCN(C(=O)c3cccs3)C(C(=O)NCc3ccc4ccccc4c3)C2)c1] | 4-[(3- methylphenyl)sulfonyl]- N-[(naphthalen-2- yl)methyl]-1- (thiophene-2- carbonyl)piperazine- 2-carboxamide | 533.7 | 534.1 |
| 89-3 | 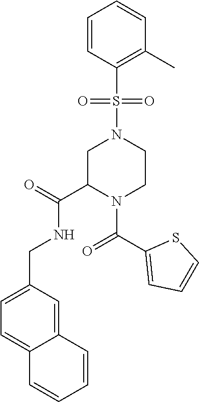 [SMILES: Cc1ccccc1S(=O)(=O)N1CCN(C(=O)c2cccs2)C(C(=O)NCc2ccc3ccccc3c2)C1] | 4-[(2- methylphenyl)sulfonyl]- N-[(naphthalen-2- yl)methyl]-1- (thiophene-2- carbonyl)piperazine-2- carboxamide | 533.7 | 534.1 |
| 89-4 | 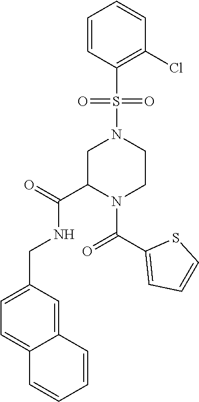 [SMILES: O=C(NCc1ccc2ccccc2c1)C1CN(S(=O)(=O)c2ccccc2Cl)CCN1C(=O)c1cccs1] | 4-[(2- chlorophenyl)sulfonyl]- N-[(naphthalen-2- yl)methyl]-1- (thiophene-2- carbonyl)piperazine- 2-carboxamide | 554.1 | 554 |
| 89-5 | 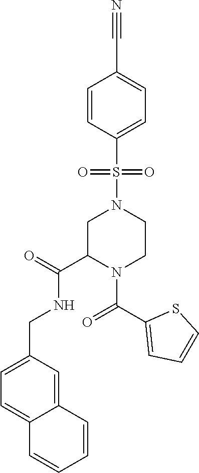 [SMILES: N#Cc1ccc(S(=O)(=O)N2CCN(C(=O)c3cccs3)C(C(=O)NCc3ccc4ccccc4c3)C2)cc1] | 4-[(4- cyanophenyl)sulfonyl]- N-[(naphthalen-2- yl)methyl]-1- (thiophene-2- carbonyl)piperazine- 2-carboxamide | 544.6 | 545.1 |
| 89-6 | 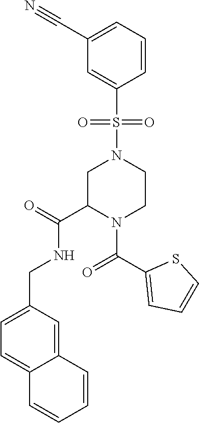 [SMILES: N#Cc1cccc(S(=O)(=O)N2CCN(C(=O)c3cccs3)C(C(=O)NCc3ccc4ccccc4c3)C2)c1] | 4-[(3- cyanophenyl)sulfonyl]- N-[(naphthalen-2- yl)methyl]-1- (thiophene-2- carbonyl)piperazine-2- carboxamide | 544.6 | 545.1 |
| 89-7 | 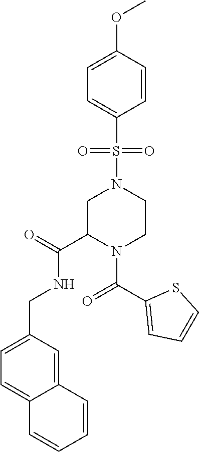 [SMILES: COc1ccc(S(=O)(=O)N2CCN(C(=O)c3cccs3)C(C(=O)NCc3ccc4ccccc4c3)C2)cc1] | 4-[(4- methoxyphenyl) sulfonyl]-N-[(naphthalen- 2-yl)methyl]-1- (thiophene-2- carbonyl)piperazine-2- carboxamide | 549.7 | 550.1 |
| 89-8 | 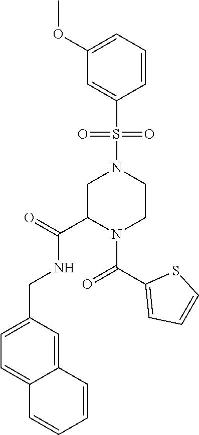 [SMILES: COc1cccc(S(=O)(=O)N2CCN(C(=O)c3cccs3)C(C(=O)NCc3ccc4ccccc4c3)C2)c1] | 4-[(3- methoxyphenyl)sulfonyl]- N-[(naphthalen-2- yl)methyl]-1- (thiophene-2- carbonyl)piperazine- 2-carboxamide | 549.7 | 550.1 |
| 89-9 | 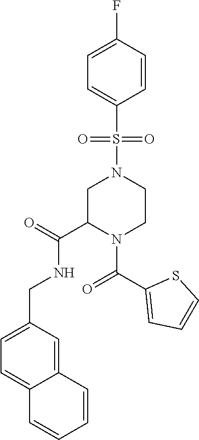 [SMILES: O=C(NCc1ccc2ccccc2c1)C1CN(S(=O)(=O)c2ccc(F)cc2)CCN1C(=O)c1cccs1] | 4-[(4- fluorophenyl)sulfonyl]- N-[(naphthalen-2- yl)methyl]-1- (thiophene-2- carbonyl)piperazine-2- carboxamide | 537.6 | 538.1 |
| 89-10 | 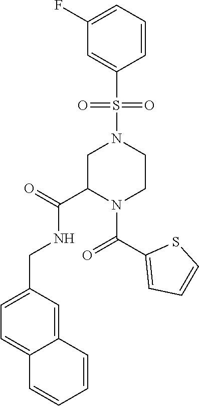 [SMILES: O=C(NCc1ccc2ccccc2c1)C1CN(S(=O)(=O)c2cccc(F)c2)CCN1C(=O)c1cccs1] | 4-[(3- fluorophenyl)sulfonyl]- N-[(naphthalen-2- yl)methyl]-1- (thiophene-2- carbonyl)piperazine- 2-carboxamide | 537.6 | 538.1 |
| 89-11 | 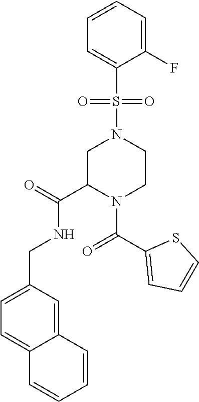 [SMILES: O=C(NCc1ccc2ccccc2c1)C1CN(S(=O)(=O)c2ccccc2F)CCN1C(=O)c1cccs1] | 4-[(2- fluorophenyl)sulfonyl]- N-[(naphthalen-2- yl)methyl]-1- (thiophene-2- carbonyl)piperazine- 2-carboxamide | 537.6 | 538.1 |

The following table shows data for representative compounds of the Examples as orexin receptor agonists as determined by assays described herein. Emax (%) represents percent activity at maximum tested concentration.

| Example | hOX2R_IP IC₅₀ (nM) | Emax (%) |
|---|---|---|
| 6 | 2257 | 94.26 |
| 10-1 | 4309 | 100.8 |
| 10-2 | 8899 | 97.41 |
| 10-3 | 12190 | 87.78 |
| 10-4 | 2607 | 98.69 |
| 10-5 | 8987 | 98.65 |
| 10-6 | 5002 | 96.79 |
| 10-7 | 1870 | 98.93 |
| 10-8 | 4669 | 89.95 |
| 10-9 | 6171 | 93.85 |
| 10-10 | 11700 | 93.94 |
| 10-11 | 100200 | 19.37 |
| 10-12 | 2433 | 89.44 |
| 11 | 34660 | 81.58 |
| 19-1 | 245.4 | 100.1 |
| 19-2 | 14.04 | 98.82 |
| 19-2-1 | 6.701 | 98.12 |
| 19-2-2 | 8469 | 87.69 |
| 19-3 | 68.94 | 87.07 |
| 19-4 | 70.24 | 101 |
| 19-4-1 | 22.73 | 100.3 |
| 19-4-2 | 9104 | 95.6 |
| 19-5 | 79.49 | 100.6 |
| 19-6 | 81.3 | 98.7 |
| 19-7 | 82.25 | 100.9 |
| 19-8 | 146.3 | 99.48 |
| 19-9 | 409.4 | 99.21 |
| 19-10 | 158.1 | 93.84 |
| 19-11 | 264.3 | 93.13 |
| 21 | 92.22 | 90.73 |
| 23 | 107.7 | 98.94 |
| 26 | 11.18 | 100.1 |
| 29 | 13.08 | 99.6 |
| 32 | 58.46 | 99.92 |
| 35 | 504.2 | 99.67 |
| 39 | 6277 | 97.8 |
| 42-1 | 120.8 | 100.1 |
| 42-2 | 1013 | 20.57 |
| 42-3 | 1013 | 4.389 |
| 47-1 | 19170 | 38.42 |
| 47-2 | 144.2 | 98.32 |
| 47-3 | 29.51 | 99.71 |
| 47-4 | 27.93 | 94.25 |
| 47-5 | 14.63 | 106.1 |
| 47-6 | 10.94 | 99.55 |
| 51 | 85.72 | 99.79 |
| 55 | 2980 | 93.47 |
| 57 | 2544 | 94.33 |
| 61 | 100 | 93.23 |
| 66 | 2227 | 97.86 |
| 71 | 244.3 | 100.4 |
| 78 | 2668 | 97.09 |
| 83 | 93.9 | 89.92 |
| 89-1 | 5718 | 60.07 |
| 89-2 | 2668 | 92.01 |
| 89-3 | 6570 | 82.69 |
| 89-4 | 5281 | 75.46 |
| 89-5 | 100200 | 23.07 |
| 89-6 | 6094 | 67.47 |
| 89-7 | 2601 | 99.72 |
| 89-8 | 8708 | 75.88 |
| 89-9 | 5509 | 89.58 |
| 89-10 | 5296 | 83.58 |
| 89-11 | 5031 | 94.5 |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

1. A compound of formula I: 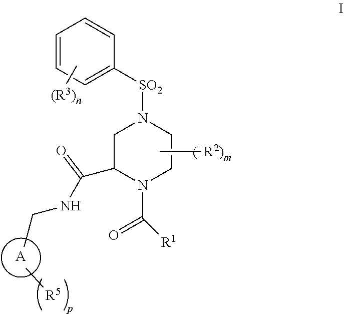
wherein:
Ring A is selected from phenyl, naphthyl and heteroaryl;
R is independently selected from H, C₁₋₆alkyl, O—C₁₋₆alkyl, CF₃, OCF₂CH₃, OCF₂CH₂CH₃, and CH₂CF₂CH₃;
R¹ is selected from:
(1) C₁₋₆ alkyl, where the alkyl is unsubstituted or substituted with one to six substituents independently selected from R⁴,
(2) C₃₋₆ cycloalkyl, where the cycloalkyl is unsubstituted or substituted with one to six substituents independently selected from R⁴,
(3) aryl, where the aryl is unsubstituted or substituted with one to three substituents independently selected from R⁴; and
(4) heteroaryl, where the heteroaryl is unsubstituted or substituted with one to three substituents independently selected from R⁴;
R² is
C₁₋₆alkyl, where the alkyl is unsubstituted or substituted with one to six substituents selected from halo, CF₃ and —C₁₋₆alkyl;
R³ is independently selected from:
(1) C₁₋₆alkyl, where the alkyl is unsubstituted or substituted with one to six substituents independently selected from R⁴,
(2) OR,
(3) halo, and
(4) CN;
R⁴ is independently selected from:
(1) hydrogen,
(2) C₁₋₆alkyl, which is unsubstituted or substituted with one to six substituents independently selected from R⁶,
(3) OR,
(4) CN,
(5) halo,
(6) aryl, which is unsubstituted or substituted with one to six substituents independently selected from R⁶, and
(7) heteroaryl, which is unsubstituted or substituted with one to six substituents independently selected from R⁶;
R⁵ is independently selected from:
(1) hydrogen,
(2) C₁₋₆alkyl, which is unsubstituted or substituted with one to six substituents independently selected from R⁶,
(3) halo,
(4) CN,
(5) aryl, which is unsubstituted or substituted with one to six substituents independently selected from R⁶,
(6) OR,
(7) heteroaryl, which is unsubstituted or substituted with one to six substituents independently selected from R⁶,
(8) OCF₃,
(9) OCF₂CH₃,
(10) OCF₂CH₂CH₃, and
(11) CH₂CF₂CH₃;
R⁶ is independently selected from: H, halo, OR, CF₃, and C₁₋₆alkyl;
m is selected from 0, 1, 2 and 3;
n is selected from 1 and 2;
p is selected from 0, 1, 2, 3, and 4;
or a pharmaceutically acceptable salt thereof.

2. The compound of claim 1, or a pharmaceutically acceptable salt thereof, wherein:
R¹ is selected from:
(1) C₁₋₆alkyl, where the alkyl is unsubstituted or substituted with one to six substituents independently selected from R⁴,
(2) phenyl, where the phenyl is unsubstituted or substituted with one to three substituents independently selected from R⁴; and
(3) -heteroaryl, where the heteroaryl is selected from thiophenyl, thiazolyl, or furanyl, which is unsubstituted or substituted with one to three substituents independently selected from R⁴;
R² is
C₁₋₆alkyl, where the alkyl is unsubstituted or substituted with one to six substituents selected from halo, CF₃ and —C₁₋₆alkyl;
R³ is independently selected from:
(1) C₁₋₆alkyl, where the alkyl is unsubstituted or substituted with one to six substituents independently selected from R⁴,
(2) OR,
(3) halo, and
(4) CN;
R⁴ is independently selected from:
(1) hydrogen,
(2) C₁₋₆alkyl, which is unsubstituted or substituted with one to six substituents independently selected from R⁶,
(3) OR,
(4) CN,
(5) halo,
(6) aryl, which is unsubstituted or substituted with one to six substituents independently selected from R⁶, and
(7) heteroaryl, which is unsubstituted or substituted with one to six substituents independently selected from R⁶;
R⁵ is independently selected from:
(1) hydrogen,
(2) C₁₋₆alkyl, which is unsubstituted or substituted with one to six substituents independently selected from R⁶,
(3) halo,
(4) CN,
(5) aryl, which is unsubstituted or substituted with one to six substituents independently selected from R⁶,
(6) OR,
(7) heteroaryl, which is unsubstituted or substituted with one to six substituents independently selected from R⁶,
(8) OCF₃,
(9) OCF₂CH₃,
(10) OCF₂CH₂CH₃, and
(11) CH₂CF₂CH₃;
R⁶ is independently selected from: H, halo, OR, CF₃, and C₁₋₆alkyl;
m is selected from 0 or 1;
n is selected from 0-1 or 2; and
p is selected from 0, 1, 2, or 3.

3. The compound of claim 1 or a pharmaceutically acceptable salt thereof, having the formula IB: 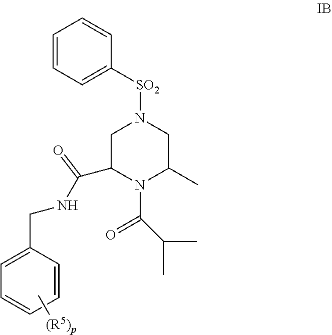
wherein:
R is independently selected from H, —C₁₋₆alkyl, O—C₁₋₆alkyl, CF₃, OCF₂CH₃, OCF₂CH₂CH₃, and CH₂CF₂CH₃;
R⁵ is independently selected from:
(1) hydrogen,
(2) C₁₋₆alkyl, which is unsubstituted or substituted with one to six substituents independently selected from R⁶,
(3) halo,
(4) CN,
(5) aryl, which is unsubstituted or substituted with one to six substituents independently selected from R⁶,
(6) OR,
(7) heteroaryl, which is unsubstituted or substituted with one to six substituents independently selected from R⁶,
(8) OCF₃,
(9) OCF₂CH₃,
(10) OCF₂CH₂CH₃, and
(11) CH₂CF₂CH₃;
R⁶ is independently selected from: H, halo, OR, CF₃, and C₁₋₆alkyl; and
p is selected from 0, 1, 2, 3, or 4.

4. The compound of claim 1 or a pharmaceutically acceptable salt thereof, wherein Ring A is selected from: phenyl, naphthyl, pyridinyl, pyrazinyl, or pyrimidinyl.

5. The compound of claim 1 or a pharmaceutically acceptable salt thereof, wherein R¹ is selected from: —C₁₋₆alkyl or phenyl, where the alkyl or phenyl is unsubstituted or substituted with one to six substituents independently selected from R⁴.

6. The compound of claim 1 or a pharmaceutically acceptable salt thereof, wherein R² is —C₁₋₆alkyl, where the alkyl is unsubstituted or substituted with one to six substituents selected from halo, CF₃ and —C₁₋₆alkyl.

7. The compound of claim 1 or a pharmaceutically acceptable salt thereof, wherein R⁵ is independently selected from C₁₋₆alkyl, phenyl, OR, heteroaryl, OCF₃, OCF₂CH₃, OCF₂CH₂CH₃, or CH₂CF₂CH₃, wherein said alkyl, phenyl or heteroaryl is unsubstituted or substituted with one to six substituents independently selected from R⁶.

8. A compound which is selected from:
N-(naphthalen-2-ylmethyl)-4-(phenylsulfonyl)-1-(thiophene-2-carbonyl)piperazine-2-carboxamide (6);
1-(2-methylbutanoyl)-N-(naphthalen-2-ylmethyl)-4-(phenylsulfonyl)piperazine-2-carboxamide (10-1);
N-(naphthalen-2-ylmethyl)-4-(phenylsulfonyl)-1-propionylpiperazine-2-carboxamide (10-2);
1-butyryl-N-(naphthalen-2-ylmethyl)-4-(phenylsulfonyl)piperazine-2-carboxamide (10-3)
1-isobutyryl-N-(naphthalen-2-ylmethyl)-4-(phenylsulfonyl)piperazine-2-carboxamide (10-4);
1-(cyclopropanecarbonyl)-N-(naphthalen-2-ylmethyl)-4-(phenylsulfonyl)piperazine-2-carboxamide (10-5);
1-(cyclobutanecarbonyl)-N-(naphthalen-2-ylmethyl)-4-(phenylsulfonyl)piperazine-2-carboxamide (10-6);
N-(naphthalen-2-ylmethyl)-4-(phenylsulfonyl)-1-(thiazole-2-carbonyl)piperazine-2-carboxamide (10-7);
N-(naphthalen-2-ylmethyl)-4-(phenylsulfonyl)-1-(thiophene-3-carbonyl)piperazine-2-carboxamide (10-8);
1-(furan-2-carbonyl)-N-(naphthalen-2-ylmethyl)-4-(phenylsulfonyl)piperazine-2-carboxamide (10-9);
N-(naphthalen-2-ylmethyl)-4-(phenylsulfonyl)-1-(thiazole-5-carbonyl)piperazine-2-carboxamide (10-10);
1-(2-methylbenzoyl)-N-(naphthalen-2-ylmethyl)-4-(phenylsulfonyl)piperazine-2-carboxamide (10-11);
1-(3-methylthiophene-2-carbonyl)-N-(naphthalen-2-ylmethyl)-4-(phenylsulfonyl)piperazine-2-carboxamide (10-12);
1-acetyl-N-(naphthalen-2-ylmethyl)-4-(phenylsulfonyl)piperazine-2-carboxamide (11);
cis-1-isobutyryl-6-methyl-N-(naphthalen-2-ylmethyl)-4-(phenylsulfonyl)piperazine-2-carboxamide (19-1);
cis-N-(4-(furan-2-yl)benzyl)-1-isobutyryl-6-methyl-4-(phenylsulfonyl)piperazine-2-carboxamide (19-2);
(2R,6R)—N-(4-(furan-2-yl)benzyl)-1-isobutyryl-6-methyl-4-(phenylsulfonyl)piperazine-2-carboxamide (19-2-1 PEAK 1);
(2S,6S)—N-(4-(furan-2-yl)benzyl)-1-isobutyryl-6-methyl-4-(phenylsulfonyl)piperazine-2-carboxamide (19-2-2, PEAK 2);
cis-N-([1,1′-biphenyl]-4-ylmethyl)-1-isobutyryl-6-methyl-4-(phenylsulfonyl)piperazine-2-carboxamide (19-3);
cis-N-(4-(1,1-difluoroethoxy)benzyl)-1-isobutyryl-6-methyl-4-(phenylsulfonyl)piperazine-2-carboxamide (19-4);
(2R,6R)—N-(4-(1,1-difluoroethoxy)benzyl)-1-isobutyryl-6-methyl-4-(phenylsulfonyl)piperazine-2-carboxamide (19-4-1, PEAK 1);
(2S,6S)—N-(4-(1,1-difluoroethoxy)benzyl)-1-isobutyryl-6-methyl-4-(phenylsulfonyl)piperazine-2-carboxamide (19-4-2, PEAK 2);
cis-1-isobutyryl-6-methyl-4-(phenylsulfonyl)-N-(4-(pyrimidin-2-yl)benzyl)piperazine-2-carboxamide (19-5);
cis-1-isobutyryl-6-methyl-4-(phenylsulfonyl)-N-(4-(thiazol-2-yl)benzyl)piperazine-2-carboxamide (19-6);
cis-N-(4-(2H-1,2,3-triazol-2-yl)benzyl)-1-isobutyryl-6-methyl-4-(phenylsulfonyl)piperazine-2-carboxamide (19-7);
cis-1-isobutyryl-6-methyl-N-(4-(oxazol-2-yl)benzyl)-4-(phenylsulfonyl)piperazine-2-carboxamide (19-8);
cis-N-(4-(1H-pyrazol-1-yl)benzyl)-1-isobutyryl-6-methyl-4-(phenylsulfonyl)piperazine-2-carboxamide (19-9);
cis-1-isobutyryl-6-methyl-4-(phenylsulfonyl)-N-(4-propylbenzyl)piperazine-2-carboxamide (19-10);
cis-N-(4-ethoxybenzyl)-1-isobutyryl-6-methyl-4-(phenylsulfonyl)piperazine-2-carboxamide (19-11);
cis-N-((2′-fluoro-[1,1′-biphenyl]-4-yl)methyl)-1-isobutyryl-6-methyl-4-(phenylsulfonyl)piperazine-2-carboxamide (21);
cis-N-((5-(furan-2-yl)pyridin-2-yl)methyl)-1-isobutyryl-6-methyl-4-(phenylsulfonyl)piperazine-2-carboxamide (23);
cis-N-(3-fluoro-4-(furan-2-yl)benzyl)-1-isobutyryl-6-methyl-4-(phenylsulfonyl)piperazine-2-carboxamide (26);
cis-N-(4-(furan-2-yl)-2-methylbenzyl)-1-isobutyryl-6-methyl-4-(phenylsulfonyl)piperazine-2-carboxamide (29);
cis-N-(2-fluoro-4-(furan-2-yl)benzyl)-1-isobutyryl-6-methyl-4-(phenylsulfonyl)piperazine-2-carboxamide (32);
cis-N-((5-(furan-2-yl)pyrazin-2-yl)methyl)-1-isobutyryl-6-methyl-4-(phenylsulfonyl)piperazine-2-carboxamide (35);
cis-N-((5-(furan-2-yl)pyrazin-2-yl)methyl)-1-isobutyryl-6-methyl-4-(phenylsulfonyl)piperazine-2-carboxamide (39);
cis-1-isobutyryl-6-methyl-4-(phenylsulfonyl)-N-(4-(pyridin-2-yl)benzyl)piperazine-2-carboxamide (42-1);
1-isobutyryl-6-methyl-4-(phenylsulfonyl)-N-(4-(pyridin-3-yl)benzyl)piperazine-2-carboxamide (42-2);
1-isobutyryl-6-methyl-4-(phenylsulfonyl)-N-(4-(pyridin-4-yl)benzyl)piperazine-2-carboxamide (42-3);
cis-N-(4-(furan-2-yl)-3-methoxybenzyl)-1-isobutyryl-6-methyl-4-(phenylsulfonyl)piperazine-2-carboxamide (47-1);
cis-N-(3-cyano-4-(furan-2-yl)benzyl)-1-isobutyryl-6-methyl-4-(phenylsulfonyl)piperazine-2-carboxamide (47-2);
cis-N-(3,5-difluoro-4-(furan-2-yl)benzyl)-1-isobutyryl-6-methyl-4-(phenylsulfonyl)piperazine-2-carboxamide (47-3);
cis-N-(4-(furan-2-yl)-2-methoxybenzyl)-1-isobutyryl-6-methyl-4-(phenylsulfonyl)piperazine-2-carboxamide (47-4);
cis-N-(5-fluoro-4-(furan-2-yl)-2-methylbenzyl)-1-isobutyryl-6-methyl-4-(phenylsulfonyl) piperazine-2-carboxamide (47-5);
cis-N-(2-ethyl-4-(furan-2-yl)benzyl)-1-isobutyryl-6-methyl-4-(phenylsulfonyl)piperazine-2-carboxamide (47-6);
cis-N-(3-fluoro-4-(furan-2-yl)-2-methylbenzyl)-1-isobutyryl-6-methyl-4-(phenylsulfonyl) piperazine-2-carboxamide (51);
cis-N-((5-(furan-2-yl)-3-methylpyridin-2-yl)methyl)-1-isobutyryl-6-methyl-4-(phenylsulfonyl)piperazine-2-carboxamide (55);
cis-6-methyl-N-(naphthalen-2-ylmethyl)-4-(phenylsulfonyl)-1-(thiophene-2-carbonyl)piperazine-2-carboxamide (57);
cis-N-(4-(1,1-difluoropropoxy)benzyl)-1-isobutyryl-6-methyl-4-(phenylsulfonyl)piperazine-2-carboxamide (61);
cis-N-(4-(1,1-difluoropropoxy)benzyl)-1-isobutyryl-6-methyl-4-(phenylsulfonyl)piperazine-2-carboxamide (66);
cis-6-ethyl-N-(4-(furan-2-yl)benzyl)-1-isobutyryl-4-(phenylsulfonyl)piperazine-2-carboxamide (71);
cis-N-(4-(furan-2-yl)benzyl)-1-isobutyryl-4-(phenylsulfonyl)-6-(trifluoromethyl)piperazine-2-carboxamide (78);
N-(4-(furan-2-yl)benzyl)-1-isobutyryl-6,6-dimethyl-4-(phenylsulfonyl)piperazine-2-carboxamide (83);
N-(naphthalen-2-ylmethyl)-1-(thiophene-2-carbonyl)-4-tosylpiperazine-2-carboxamide (89-1);
4-[(3-methylphenyl)sulfonyl]-N-[(naphthalen-2-yl)methyl]-1-(thiophene-2-carbonyl)piperazine-2-carboxamide (89-2);
4-[(2-methylphenyl)sulfonyl]-N-[(naphthalen-2-yl)methyl]-1-(thiophene-2-carbonyl)piperazine-2-carboxamide (89-3);
4-[(2-chlorophenyl)sulfonyl]-N-[(naphthalen-2-yl)methyl]-1-(thiophene-2-carbonyl)piperazine-2-carboxamide (89-4);
4-[(4-cyanophenyl)sulfonyl]-N-[(naphthalen-2-yl)methyl]-1-(thiophene-2-carbonyl)piperazine-2-carboxamide (89-5);
4-[(3-cyanophenyl)sulfonyl]-N-[(naphthalen-2-yl)methyl]-1-(thiophene-2-carbonyl)piperazine-2-carboxamide (89-6);
4-[(4-methoxyphenyl)sulfonyl]-N-[(naphthalen-2-yl)methyl]-1-(thiophene-2-carbonyl)piperazine-2-carboxamide (89-7);
4-[(3-methoxyphenyl)sulfonyl]-N-[(naphthalen-2-yl)methyl]-1-(thiophene-2-carbonyl)piperazine-2-carboxamide (89-8);
4-[(4-fluorophenyl)sulfonyl]-N-[(naphthalen-2-yl)methyl]-1-(thiophene-2-carbonyl)piperazine-2-carboxamide (89-9);
4-[(3-fluorophenyl)sulfonyl]-N-[(naphthalen-2-yl)methyl]-1-(thiophene-2-carbonyl)piperazine-2-carboxamide (89-10); and
4-[(2-fluorophenyl)sulfonyl]-N-[(naphthalen-2-yl)methyl]-1-(thiophene-2-carbonyl)piperazine-2-carboxamide (89-11);
or a pharmaceutically acceptable salt thereof.

9. The compound of claim 8 which is selected from:
cis-N-(4-(furan-2-yl)benzyl)-1-isobutyryl-6-methyl-4-(phenylsulfonyl)piperazine-2-carboxamide (19-2);
(2R,6R)—N-(4-(furan-2-yl)benzyl)-1-isobutyryl-6-methyl-4-(phenylsulfonyl)piperazine-2-carboxamide (19-2-1 PEAK 1);
cis-N-([1,1′-biphenyl]-4-ylmethyl)-1-isobutyryl-6-methyl-4-(phenylsulfonyl)piperazine-2-carboxamide (19-3);
cis-N-(4-(1,1-difluoroethoxy)benzyl)-1-isobutyryl-6-methyl-4-(phenylsulfonyl)piperazine-2-carboxamide (19-4);
(2R,6R)—N-(4-(1,1-difluoroethoxy)benzyl)-1-isobutyryl-6-methyl-4-(phenylsulfonyl)piperazine-2-carboxamide (19-4-1, PEAK 1);
cis-1-isobutyryl-6-methyl-4-(phenylsulfonyl)-N-(4-(pyrimidin-2-yl)benzyl)piperazine-2-carboxamide (19-5);
cis-1-isobutyryl-6-methyl-4-(phenylsulfonyl)-N-(4-(thiazol-2-yl)benzyl)piperazine-2-carboxamide (19-6);
cis-N-(4-(2H-1,2,3-triazol-2-yl)benzyl)-1-isobutyryl-6-methyl-4-(phenylsulfonyl)piperazine-2-carboxamide (19-7);
cis-1-isobutyryl-6-methyl-N-(4-(oxazol-2-yl)benzyl)-4-(phenylsulfonyl)piperazine-2-carboxamide (19-8);
cis-1-isobutyryl-6-methyl-4-(phenylsulfonyl)-N-(4-propylbenzyl)piperazine-2-carboxamide (19-10);
cis-N-((2′-fluoro-[1,1′-biphenyl]-4-yl)methyl)-1-isobutyryl-6-methyl-4-(phenylsulfonyl)piperazine-2-carboxamide (21); and
cis-1-isobutyryl-6-methyl-4-(phenylsulfonyl)-N-(4-(pyridin-2-yl)benzyl)piperazine-2-carboxamide (42-1);
or a pharmaceutically acceptable salt thereof.

10. A pharmaceutical composition which comprises an inert carrier and a compound of claim 1 or a pharmaceutically acceptable salt thereof.

11-12. (canceled)

13. A method for treating narcolepsy in a mammalian subject which comprises administering to the patient an effective amount of the compound of claim 1 or a pharmaceutically acceptable salt thereof.

14. A method for treating hypersomnia in a mammalian subject which comprises administering to the patient an effective amount of the compound of claim 1 or a pharmaceutically acceptable salt thereof.

15. A compound of formula I: 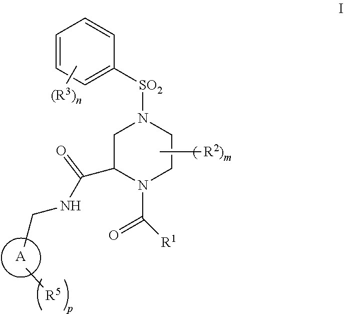
wherein:
Ring A is selected from naphthyl and heteroaryl;
R is independently selected from H, C₁₋₆alkyl, O—C₁₋₆alkyl, CF₃, OCF₂CH₃, OCF₂CH₂CH₃, and CH₂CF₂CH₃;
R¹ is selected from:
(1) C₁₋₆ alkyl, where the alkyl is unsubstituted or substituted with one to six substituents independently selected from R⁴,
(2) C₃₋₆ cycloalkyl, where the cycloalkyl is unsubstituted or substituted with one to six substituents independently selected from R⁴,
(3) aryl, where the aryl is unsubstituted or substituted with one to three substituents independently selected from R⁴; and
(4) heteroaryl, where the heteroaryl is unsubstituted or substituted with one to three substituents independently selected from R⁴;
R² is H;
R³ is independently selected from:
(1) C₁₋₆alkyl, where the alkyl is unsubstituted or substituted with one to six substituents independently selected from R⁴,
(2) OR,
(3) halo, and
(4) CN;
R⁴ is independently selected from:
(1) hydrogen,
(2) C₁₋₆alkyl, which is unsubstituted or substituted with one to six substituents independently selected from R⁶,
(3) OR,
(4) CN,
(5) halo,
(6) aryl, which is unsubstituted or substituted with one to six substituents independently selected from R⁶, and
(7) heteroaryl, which is unsubstituted or substituted with one to six substituents independently selected from R⁶;
R⁵ is independently selected from:
(1) hydrogen,
(2) C₁₋₆alkyl, which is unsubstituted or substituted with one to six substituents independently selected from R⁶,
(3) halo,
(4) CN,
(5) aryl, which is unsubstituted or substituted with one to six substituents independently selected from R⁶,
(6) OR,
(7) heteroaryl, which is unsubstituted or substituted with one to six substituents independently selected from R⁶,
(8) OCF₃,
(9) OCF₂CH₃,
(10) OCF₂CH₂CH₃, and
(11) CH₂CF₂CH₃;
R⁶ is independently selected from: H, halo, OR, CF₃, and C₁₋₆alkyl;
m is selected from 0, 1, 2 and 3;
n is selected from 0, 1 and 2;
p is selected from 0, 1, 2, 3, and 4;
or a pharmaceutically acceptable salt thereof.

16. A pharmaceutical composition which comprises an inert carrier and a compound of claim 15 or a pharmaceutically acceptable salt thereof.

17. A method for treating narcolepsy in a mammalian subject which comprises administering to the patient an effective amount of the compound of claim 15 or a pharmaceutically acceptable salt thereof.

18. A method for treating hypersomnia in a mammalian subject which comprises administering to the patient an effective amount of the compound of claim 15 or a pharmaceutically acceptable salt thereof.
